SEQUENCE LISTING (67 sequences)

SEQ ID NO: 1 - DNA, 1029 bp, Homo sapiens
atggacctgtggaactgggatgaggcatccccacaggaagtgcctccagggaacaagctggcagggcttgaaggagccaaattaggcttctgtttccctgatctggcactccaaggggacacgccgacagcgacagcagagacatgctggaaaggtacaagctcatccctggcaagcttcccacagctggactggggctccgcgttactgcacccagaagttccatggggggcggagcccgactctcaggctcttccgtggtccggggactggacagacatggcgtgcacagcctgggactcttggagcggcgcctcgcagaccctgggccccgcccctctcggcccgggccccatccccgccgccggctccgaaggcgccgcgggccagaactgcgtccccgtggcgggagaggccacctcgtggtcgcgcgcccaggccgccgggagcaacaccagctgggactgttctgtggggcccgacggcgatacctactggggcagtggcctgggcggggagccgcgcacggactgtaccatttcgtggggcgggcccgcgggcccggactgtaccacctcctggaacccggggctgcatgcgggtggcaccacctctttgaagcggtaccagagctcagctctcaccgtttgctccgaaccgagcccgcagtcggaccgtgccagtttggctcgatgccccaaaactaaccaccgaggtcccattcagctgtggcagttcctcctggagctgctccacgacggggcgcgtagcagctgcatccgttggactggcaacagccgcgagttccagctgtgcgaccccaaagaggtggctcggctgtggggcgagcgcaagagaaagccgggcatgaattacgagaagctgagccggggccttcgctactactatcgccgcgacatcgtgcgcaagagcggggggcgaaagtacacgtaccgcttcgggggccgcgtgcccagcctagcctatccggactgtgcgggaggcggacggggagcagagacacaataa

SEQ ID NO: 2 - DNA, 38 bp, Artificial
  portion of 3rd exon of KDR
  misc_feature (21)..(21): n is a, c, g, or t
agtgcttggcctctgactttnatgcctatgtttatggc

SEQ ID NO: 3 - DNA, 50 bp, Artificial
  edited allele 1
tgacactggagcctacaagtgcttctaccgggaaactgacttggcctcgg

SEQ ID NO: 4 - DNA, 49 bp, Artificial
  edited allele 2
tgacactggagcctacaagtgcttctacgggaaactgacttggcctcgg

SEQ ID NO: 5 - DNA, 37 bp, Artificial
  portion of 4th exon of ETV2
  misc_feature (29)..(30): n is a, c, g, or t
gtacagtgcggggctccccctccccgccnntgcccct

SEQ ID NO: 6 - DNA, 61 bp, Artificial
  edited allele 1
ggggagccgcgcacggactgtaccatttcgtggggcgggcccgcgggcccggactgtacca

SEQ ID NO: 7 - DNA, 61 bp, Artificial; identical to SEQ ID NO: 6
  edited allele 2

SEQ ID NO: 8 - DNA, 20 bp, Artificial
  sequence from control IPSC
aaccttaatggtatgtaatt

SEQ ID NO: 9 - DNA, 20 bp, Artificial
  sequence from patient IPSC
aaccttaatgatatgtaatt

SEQ ID NO: 10 - DNA, 20 bp, Artificial
  ETV2 gRNA sequence
acggactgtaccatttcgtg

SEQ ID NO: 11 - DNA, 20 bp, Artificial
  ETV2 PCR forward primer
cactcgggatccgttactcc

SEQ ID NO: 12 - DNA, 20 bp, Artificial
  ETV2 PCR reverse primer
gttcggagcaaacggtgaga

SEQ ID NO: 13 - DNA, 20 bp, Artificial
  ETV2 Sanger sequencing primer
gttcggagcaaacggtgaga

SEQ ID NO: 14 - DNA, 20 bp, Artificial
  KDR gRNA sequence
gagcctacaagtgcttctac

SEQ ID NO: 15 - DNA, 25 bp, Artificial
  KDR PCR forward primer
caagccctttgttgtactcaattct

SEQ ID NO: 16 - DNA, 25 bp, Artificial
  KDR PCR reverse primer
attaatttttcaggggacagaggga

SEQ ID NO: 17 - DNA, 25 bp, Artificial
  KDR Sanger sequencing primer
attaatttttcaggggacagaggga

SEQ ID NO: 18 - DNA, 22 bp, Artificial
  qRT-PCR primer POU5F1 forward
gggctctcccatgcattcaaac

SEQ ID NO: 19 - DNA, 22 bp, Artificial
  qRT-PCR primer POU5F1 reverse
caccttccctccaaccagttgc

SEQ ID NO: 20 - DNA, 21 bp, Artificial
  qRT-PCR primer MIXL1 forward
acgtctttcagcgccgaacag

SEQ ID NO: 21 - DNA, 21 bp, Artificial
  qRT-PCR primer MIXL1 reverse
ttggttcgggcaggcagttca

SEQ ID NO: 22 - DNA, 26 bp, Artificial
  qRT-PCR primer TBXT forward
gtgctgtcccaggtggcttacagatg

SEQ ID NO: 23 - DNA, 27 bp, Artificial
  qRT-PCR primer TBXT reverse
ccttaacagctcaactctaactacttg

SEQ ID NO: 24 - DNA, 24 bp, Artificial
  qRT-PCR primer ACTA2 forward
tgacaatggctctgggctctgtaa

SEQ ID NO: 25 - DNA, 24 bp, Artificial
  qRT-PCR primer ACTA2 reverse
ttcgtcacccacgtagctgtcttt

SEQ ID NO: 26 - DNA, 24 bp, Artificial
  qRT-PCR primer GAPDH forward
catgttcgtcatgggtgtgaacca

SEQ ID NO: 27 - DNA, 24 bp, Artificial
  qRT-PCR primer GAPDH reverse
atggcatggactgtggtcatgagt

SEQ ID NO: 28 - DNA, 26 bp, Artificial
  qRT-PCR primer ERG forward
aaccatctccttccacagtgcccaaa

SEQ ID NO: 29 - DNA, 24 bp, Artificial
  qRT-PCR primer ERG reverse
tttgcaaggcggctacttgttggt

SEQ ID NO: 30 - DNA, 19 bp, Artificial
  qRT-PCR primer ETV2 forward
ccgacggcgatacctactg

SEQ ID NO: 31 - DNA, 21 bp, Artificial
  qRT-PCR primer ETV2 reverse
cggtggttagttttggggcat

SEQ ID NO: 32 - DNA, 24 bp, Artificial
  qRT-PCR primer NOS3 forward
tgaccctcaccgctacaacatcct

SEQ ID NO: 33 - DNA, 26 bp, Artificial
  qRT-PCR primer NOS3 reverse
cgttgatttccactgctgccttgtct

SEQ ID NO: 34 - DNA, 20 bp, Artificial
  qRT-PCR primer CLDN5 forward
ctctgctggttcgccaacat

SEQ ID NO: 35 - DNA, 20 bp, Artificial
  qRT-PCR primer CLDN5 reverse
cagctcgtacttctgcgaca

SEQ ID NO: 36 - DNA, 22 bp, Artificial
  qRT-PCR primer ENG forward
cggtggtcaatatcctgtcgag

SEQ ID NO: 37 - DNA, 22 bp, Artificial
  qRT-PCR primer ENG reverse
aggaagtgtgggctgaggtaga

SEQ ID NO: 38 - DNA, 22 bp, Artificial
  qRT-PCR primer TEK forward
gcttgctcctttctggaactgt

SEQ ID NO: 39 - DNA, 17 bp, Artificial
  qRT-PCR primer TEK reverse
cgccacccagaggcaat

SEQ ID NO: 40 - DNA, 19 bp, Artificial
  qRT-PCR primer PECAM1 forward
cacctggcccaggagtttc

SEQ ID NO: 41 - DNA, 23 bp, Artificial
  qRT-PCR primer PECAM1 reverse
agtacacagccttgttgccatgt

SEQ ID NO: 42 - DNA, 22 bp, Artificial
  qRT-PCR primer CDH5 forward
gaacccaagatgtggcctttag

SEQ ID NO: 43 - DNA, 23 bp, Artificial
  qRT-PCR primer CDH5 reverse
gatgtgacaacagcgaggtgtaa

SEQ ID NO: 44 - DNA, 21 bp, Artificial
  qRT-PCR primer VWF forward
gtcgagctgcacagtgacatg

SEQ ID NO: 45 - DNA, 22 bp, Artificial
  qRT-PCR primer VWF reverse
gcaccataaacgttgacttcca

SEQ ID NO: 46 - DNA, 24 bp, Artificial
  qRT-PCR primer KDR forward
atccagtgggctgatgaccaagaa

SEQ ID NO: 47 - DNA, 24 bp, Artificial
  qRT-PCR primer KDR reverse
accagagattccatgccacttcca

SEQ ID NO: 48 - DNA, 53 bp, Artificial
  PCR primer F8 attB Forward
ggggacaagtttgtacaaaaaagcaggcttaatgcaaatagagctctccacct

SEQ ID NO: 49 - DNA, 52 bp, Artificial
  PCR primer F8 attB Reverse
ggggaccactttgtacaagaaagctgggtttcagtagaggtcctgtgcctcg

SEQ ID NO: 50 - DNA, 20 bp, Artificial
  PCR primer BDD vs FL gel P1
agactttcggaacagaggca

SEQ ID NO: 51 - DNA, 20 bp, Artificial
  PCR primer BDD vs FL gel P2
ttctgtgtgcaaaccaaggg

SEQ ID NO: 52 - DNA, 20 bp, Artificial
  PCR primer BDD vs FL gel P3
ggcaaagcaaggtaggactg

SEQ ID NO: 53 - DNA, 22 bp, Artificial
  PCR primer BDD vs FL gel P4
gagccctgtttcttagaacatg

SEQ ID NO: 54 - DNA, 21 bp, Artificial
  PCR primer Exon 22 FWD1
gtggatctgttggcaccaatg

SEQ ID NO: 55 - DNA, 20 bp, Artificial
  PCR primer Exon 24 REV
ctcccttggaggtgaagtcg

SEQ ID NO: 56 - DNA, 26 bp, Artificial
  PCR primer Exon 22 FWD2
accaatgattattcacggcatcaaga

SEQ ID NO: 57 - DNA, 25 bp, Artificial
  PCR primer Exon 23 REV
tgcaaacggatgtatcgagcaataa

SEQ ID NO: 58 - DNA, 20 bp, Artificial
  PCR primer Exon 19 FWD1
tccaaagctggaatttggcg

SEQ ID NO: 59 - DNA, 20 bp, Artificial
  PCR primer Intron 22 REV
acacagtcctgaatcacata

SEQ ID NO: 60 - DNA, 20 bp, Artificial
  qPCR primer F8-P1
ccagaatcagcaaggtggat

SEQ ID NO: 61 - DNA, 20 bp, Artificial
  qPCR primer F8-P1
aggtttctgctgcttggaaa

SEQ ID NO: 62 - DNA, 20 bp, Artificial
  qPCR primer F8-P2
cactcttcgcatggagttga

SEQ ID NO: 63 - DNA, 20 bp, Artificial
  qPCR primer F8-P2
agtccacttgcagccactct

SEQ ID NO: 64 - DNA, 21 bp, Artificial
  qPCR primer SPT
cagagaaccatcagaggcaag

SEQ ID NO: 65 - DNA, 20 bp, Artificial
  qPCR primer SPT
tcaccaggatgccgaagaag

SEQ ID NO: 66 - DNA, 24 bp, Artificial
  qPCR primer GAPDH
catgttcgtcatgggtgtgaacca

SEQ ID NO: 67 - DNA, 24 bp, Artificial
  qPCR primer GAPDH
atggcatggactgtggtcatgagt

(19) United States
(12) Patent Application Publication
(10) Pub. No.: US 2020/0385685 A1
(43) Pub. Date: Dec. 10, 2020

(54) ROBUST DIFFERENTIATION OF HUMAN PLURIPOTENT STEM CELLS INTO ENDOTHELIAL CELLS USING TRANSCRIPTION FACTOR ETV2

(71) Applicant: Children`s Medical Center Corporation, Boston, MA (US)

(72) Inventors: Kai Wang, Brookline, MA (US); Ruei-Zeng Lin, Brookline, MA (US); Juan M. Melero-Martin, Bedford, MA (US)

(21) Appl. No.: 16/885,999

(22) Filed: May 28, 2020

Related U.S. Application Data
(60) Provisional application No. 62/853,655, filed on May 28, 2019.

(51) Int. Cl. C12N 5/071; A61K 35/44

(52) U.S. Cl. CPC C12N 5/0692; A61K 35/44; C12N 2506/45; C12N 2501/11; C12N 2501/165; C12N 2501/115; C12N 2501/15

(57) ABSTRACT

Methods for in vitro differentiation of human pluripotent stem cells into endothelial cells, using a protocol that includes transient expression of exogenous ETS translocation variant 2 (ETV2), and uses of those cells in human therapies, e.g., to treat hemophilia.

### CLAIM OF PRIORITY

This application claims priority under 35 USC § 119(e) to U.S. Provisional Patent Application Ser. No. 62/853,655, filed on May 28, 2019. The entire contents of the foregoing are hereby incorporated by reference.

### FEDERALLY SPONSORED RESEARCH OR DEVELOPMENT

This invention was made with Government support under Grant Nos. AR069038, HL128452, and AI123883 awarded by the National Institutes of Health. The Government has certain rights in the invention.

### TECHNICAL FIELD

Described herein are methods for in vitro differentiation of human pluripotent stem cells into endothelial cells, using a protocol that includes transient expression of exogenous ETS translocation variant 2 (ETV2).

### BACKGROUND

Endothelial cells (ECs) are implicated in the pathogenesis of numerous diseases particularly due to their ability to modulate the activity of various stem cells during tissue homeostasis and regeneration^{1,2}. Consequently, deriving competent ECs is central to many efforts in regenerative medicine.

### SUMMARY

Described herein are methods for in vitro differentiation of human pluripotent stem cells into endothelial cells, using a protocol that includes transient expression of exogenous ETS translocation variant 2 (ETV2). Further, described herein are ex vivo gene therapy methods that use hemophilia A patients' own cells, e.g., to create an implantable graft capable of delivering full-length FVIII directly into the bloodstream. The approach is based on the concept of bioengineering a vascular network in which the endothelium is lined by patients' cells that are genetically-engineered to carry out a drug delivery role. Thus, provided herein are methods for generating induced endothelial cells. The methods include providing a population of induced pluripotent stem cells (iPSCs) or human embryonic stem cells (h-ES cells); incubating the iPSCs in media in the presence of a GSK3 inhibitor, under conditions sufficient for the iPSC to differentiate into intermediate mesodermal progenitor cells (MPCs); optionally dissociating the MPCs into single cells; introducing an exogenous nucleic acid encoding ETS translocation variant 2 (ETV2) to the MPCs to induce transient expression of exogenous ETV2; and maintaining the MPCs under conditions sufficient for the MPCs to differentiate into iPSCs. The transient expression of exogenous ETV2 occurs in the MPCs (not in the iPSCs, and not later).

In some embodiments, the GSK3 inhibitor is CHIR99021, BIO, NP031112, IM-12; a pyrazolopyrimidine derivative, an analog of 7-hydroxy-1H-benzimidazole, a pyridinone (e.g., 4-(4-hydroxy-3-methylphenyl)-6-phenyl pyrimidin-2-ol), a pyrimidine, an indolylmaleimide analog, an imidazopyridine, an oxadiazole, a pyrazine, a thiadiazolidinone, amodin or 4-aminoethylamino emodin, or a 5-Imino-1,2,4-Thiadiazole (ITDZ).

In some embodiments, the iPSCs are incubated in in the presence of the GSK3 inhibitor for about 48 hours.

In some embodiments, the MPCs are incubated in media comprising (i) one or more growth factors, preferably selected from the group consisting of VEGF-A, FGF-2, and EGF, and (i) a TGFbeta receptor antagonist.

In some embodiments, the TGFbeta receptor antagonist is selected from the goup consisting of galunisertib (LY2157299 Monohydrate); A 83-01; RepSox; SD 208; SB 505124; LY 364947; D 4476; SB 525334; GW 788388; R 268712; IN 1130; SM 16; A 77-01; and SB431542. See, e.g., de Gramont, Oncoimmunology. 2017; 6(1): e1257453. In some embodiments, the MPCs are incubated in the media for about 48 hours after introduction of the ETV2 nucleic acid.

In some embodiments, the ETV2 nucleic acid comprises or encodes a sequence that is at least 95% identical to SEQ ID NO:1.

In some embodiments, the ETV2 nucleic acid is a synthetic, chemically modified mRNA, wherein at least one pseudouridine is substituted for uridine and/or at least one 5-methyl-cytosine is substituted for cytosine.

In some embodiments, the iPSCs are derived from a human primary cell.

In some embodiments, the method include comprising maintaining the iECs in culture under conditions to allow for cell proliferation.

Also provided herein are populations of iECs made by a method described herein.

Additionally provided herein are methods for treating a subject in need of vascular cell therapy that include administering to the subject a therapeutically effective amount of a population of iECs made by a method described herein.

In some embodiments, the subject is in need of vascular cell therapy to treat ischemic or vascular injury and/or endothelial denudation, e.g., in limbs, retina or myocardium; or for revascularization/neovascularization, e.g., to treat diabetes or promote success after organ transplantation.

Also provided are methods for treating a subject who has hemophilia A or hemophilia B.

The methods include administering to the subject a therapeutically effective amount of a population of iECs made by a method described herein, preferably wherein the iECs have been engineered to express Factor VIII or Factor IX.

In some embodiments, the cells are administered to the subject in a hydrogel.

In some embodiments, the hydrogel is administered by subcutaneous implantation.

Also provided herein are compositions comprising a hydrogel and a population of iECs made by a method described herein.

In some embodiments, the iECs have been engineered to express an exogenous protein.

In some embodiments, the exogenous protein is Factor VIII (to treat hemophilia A) or Factor IX (to treat hemophilia B).

In some embodiments, the hydrogel comprises collagen and/or fibrin, e.g., is a collagen/fibrin hydrogel or a crosslinked collagen hydrogel.

In some embodiments, engineering the cells to express a protein comprises introducing into the iECs a vector, preferably a transposon vector, for expression of the exogenous protein, e.g., of Factor VIII or Factor IX.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIGS. 1A-G|Robust endothelial differentiation of h-iPSCs. (a) Schematic of optimized two-stage endothelial differentiation protocol. Stage 1: conversion of h-iPSCs into h-MPCs mediated by the GSK-3 inhibitor CHIR99021. Stage 2: transfection of h-MPCs with modRNA encoding ETV2 and culture in chemically defined medium. (b) Conversion efficiency of h-iPSCs into VE-Cadherin+/CD31+ h-iECs by flow cytometry. Time course comparison of the standard S1-S2 and the optimized S1-modETV2 protocols. (c) Effect of modRNA concentration on h-iPSC-to-h-iEC conversion efficiency at 96 h. Titration analysis of CD31+ cells by flow cytometry for both electroporation- and lipofection-based delivery of modRNA. (d) Time course immunofluorescence staining for ETV2 (red) and CD31 (green) in S1-S2 and S1-modETV2 protocols. Nuclei stained by DAPI. Scale bar, 100 μm. (e) Flow cytometry analysis of differentiation efficiency at 96 h in 13 h-iPSC clones generated from dermal fibroblasts (FB), umbilical cord blood-derived ECFCs (cbECFC), and urine-derived epithelial cells (uEP). (f) Differences in differentiation efficiency between S1-S2 and S1-modETV2 protocols for all 13 h-iPSC clones. Data correspond to percentage of CD31+ cells by flow cytometry. (g) Differences in differentiation efficiency between four alternative S1-S2 methodologies and the S1 -modETV2 protocol for 3 independent h-iPSC clones. Bars represent mean±s.d.; ***P<0.001.

FIGS. 2A-F|Inefficient activation of endogenous ETV2 in intermediate h-MPCs during the standard S1-S2 differentiation protocol. (a) Time course analysis of mRNA expression (qRT-PCR) of transcription factors TEXT (mesodermal commitment) and ETV2 (endothelial commitment) during the standard S1-S2 differentiation protocol. Relative fold change normalized to GAPDH expression. (b) Immunofluorescence staining for Brachyury in h-iPSCs at 48 h during the S1-S2 protocol. h-iPSCs lacking endogenous ETV2 (h-iPSCs-ETV2^{-/-}) served as control. Nuclei stained by DAPI. Scale bar, 100 μm. Percentage of Brachyury+ cells at day 1. (c) Immunofluorescence staining for ETV2 in h-iPSCs at 72 h during the S1-S2 protocol. h-iPSCs-ETV2^{-/-} served as control. Nuclei stained by DAPI. Scale bar, 100 μm. Percentage of ETV2+ cells at day 3. (d) Effect of VEGF-A concentration on the percentages of ETV2+ cells at 72 h and CD31+ cells at 96 h during the S1-S2 protocol measured by immunofluorescence staining (ETV2) and flow cytometry (CD31). (e) Immunofluorescence staining for ETV2 in h-iPSCs during the optimized S1-modETV2 protocol. h-iPSCs-ETV2^{-/-} served as control. Nuclei stained by DAPI. Scale bar, 100 μm. Percentage of ETV2+ cells after transfection with modRNA. (f) Conversion efficiency of h-iPSCs into CD31+ h-iECs by flow cytometry. Comparison of the standard S1-S2 and the S1-modETV2 protocols. h-iPSCs-ETV2^{-/-}, h-iPSCs-KDR^{-/-}, and h-iPSCs treated with the VEGFR2 inhibitor SU5416 served as controls. In panels b, c, and e, bars represent mean±s.d.; n=4; n.s.=no statistical differences and ***P<0.001 between h-iPSCs and h-iPSCs-ETV2^{-/-}. In panel f, n=3; ***P<0.001 between indicated groups.

FIGS. 3A-H Transcriptional analysis of h-iECs obtained from various differentiation protocols. (a) Schematic of protocol for early transfection of h-iPSCs with modRNA encoding ETV2 (b) Conversion efficiency h-iPSCs into CD31+ cells by flow cytometry using the early modETV2 protocol. (c-h) RNAseq analysis across multiple h-iECs samples generated from three independent h-iPSC lines using all three differentiation protocols. Human ECFCs and the parental undifferentiated h-iPSCs served as positive and negative controls, respectively. (c) Number of differentially expressed genes between h-iECs samples from each differentiation protocol. (d) Pairwise correlation based on Pearson coefficients between all samples. (e) Principal component analysis. (f) Heatmap and hierarchical clustering analysis of global differentially expressed genes. (g) Heatmap and hierarchical clustering analysis of selected EC-specific genes. (h) GO analysis between h-iECs generated with the S1-modETV2 and the early modETV2 differentiation protocols. Analysis carried out with differentially expressed genes from EC clusters #5 and #10 based on (f). Genes displayed correspond to positive enrichment for h-iECs generated with the S1-modETV2 protocol.

FIGS. 4A-H|Functional properties of h-iECs. (a) Expansion potential of h-iECs derived by the standard S1-S2, optimized S1-modETV2, and early modETV2 protocols. Cumulative cell number measured over time in serially passaged h-iECs, starting with 10⁶ h-iPSCs. All h-iECs were purified as CD31+ cells at day 4. (b) Capillary-like networks formed by h-iECs on Matrigel. Live cells stained by Calcein-AM. Scale bar, 200 μm. The ability to form capillary-like networks was quantified and expressed as total number of branches per mm². (c) Sprouts formation by spheroids formed with h-iECs^{GFP} and h-MSCs and embedded in fibrin gel for 4 days. The ability to form lumenal sprouts was quantified and expressed as total length per field. Scale bar, 200 μm. (d) Induction of smooth muscle cell differentiation of h-MSCs by h-iECs. Representative immunofluorescent images of h-MSCs in the absence or presence of h-iECs. Differentiation was assessed by the expression of smooth muscle myosin heavy chain 11 (MYH11). Expression of VE-Cadherin was used to detect h-iECs and DAPI for cell nuclei. Scale bar, 100 μm. Quantification of smooth muscle myogenic differentiation as number of h-MSCs expressing MYH11 per unit of culture area. (e) Nitric oxide (NO) production by h-iECs detected by flow cytometry as mean fluorescence intensity upon exposure to 4-amino-5-methylamino-2′,7′-difluorofluorescein diacetate (DAF-FM). Cells without exposure to DAF-FM served as negative control. (f) Upregulation of leukocyte adhesion molecules ICAM-1, E-Selectin, and VCAM-1 in h-ECs measured as mean fluorescence intensity by flow cytometry upon exposure to TNF-α. Cells not exposed to TNF-α served as negative control. (g) Representative brightfield images of h-iECs with an increased number of bound leukocytes after TNF-α treatment. Scale bar, 50 μm. Quantification of bound leukocytes per mm² upon exposure to TNF-α. Cells without exposure to TNF-α served as negative control. (h) Capacity of h-iEC to align in the direction of flow. Representative immunofluorescent images of h-iECs under static and flow conditions. Cells stained by CD31 and nuclei by DAPI. Scale bar, 100 μm. Cell alignment quantified as frequency of cell orientation angle in histogram plots. 0° represents the direction of flow. In all panels, bars represent mean±s.d.; n=3; *P<0.05, **P<0.01, ***P<0.001 between h-iECs and ECFCs. ^{#}P<0.05, ^{##}P<0.01, ^{###}P<0.001 compared to h-iECs generated by S1-S2 protocol. n.s.=no statistical differences compared to both ECFCs and h-iECs generated by S1-S2 protocol.

FIGS. 5A-I In vivo vascular network-forming ability of h-iECs. (a) Schematic of microvascular graft models. Grafts were prepared by combining h-iECs with h-MSCs in hydrogels and were then subcutaneously implanted into SCID mice for 7 days. Grafts contained h-iECs that were generated by either the S1-modETV2 or the early modETV2 protocol. Images are macroscopic views of the explanted grafts at day 7. (b) Haematoxylin and eosin (H&E) staining of explanted grafts after 7 days in vivo. Perfused vessels were identified as luminal structures containing red blood cells (RBCs) (yellow arrowheads). Blue arrowheads represent unperfused luminal structures. (c) Density of perfused blood vessels on day 7. Groups include grafts with h-iECs that were generated by either the standard S1-S2, the optimized S1-modETV2, or the early modETV2 protocol. Grafts with ECFCs served as control. (d-e) Immunofluorescence staining of explanted grafts after 7 days in vivo. Human lumens stained by (d) UEA-1 and (e) h-CD31. Perivascular coverage stained by α-SMA. Nuclei stained by DAPI. Scale bar, 100 μm. Grafts with h-iECs from the S1-modETV2 protocol had an extensive network of α-SMA-invested human lumens, whereas grafts with h-iECs from the early modETV2 protocol presented human lumens with no perivascular coverage. (f) Percentage of human lumens with α-SMA+ perivascular coverage in explanted grafts at day 7. Groups include grafts with h-iECs that were generated by either the standard S1-S2, the optimized S1-modETV2, or the early modETV2 protocol. Grafts with ECFCs served as control. (g) Immunofluorescence staining by TUNEL and h-CD31 of explanted grafts after 7 days in vivo. Nuclei stained by DAPI. Scale bar, 100 μm. Percentage of human lumens that were TUNEL+ in explanted grafts at day 7. Groups include grafts with h-iECs generated by the S1-modETV2 and the early modETV2 protocols. Bars represent mean±s.d.; n=5; **P<0.01. (h) Grafts with h-iECs generated by the S1-modETV2 protocol were explanted at day 30. Images are macroscopic views of the explanted grafts. Immunofluorescence staining of explanted grafts by h-CD31 and α-SMA. Nuclei stained by DAPI. Scale bar, 500 μm. (i) Schematic of in vivo vascular network-forming ability of h-iECs generated by either the optimized S1-modETV2 or the early modETV2 protocols. In c and f, bars represent mean±s.d.; n=5; ***P<0.001 between h-iECs and ECFCs. ^{###}P<0.001 compared to h-iECs generated by S1-S2 protocol. n.s.=no statistical differences compared to both ECFCs and h-iECs generated by S1-S2 protocol.

FIGS. 6A-B|Derivation of h-iECs from h-iPSCs with modRNA. (a) Schematic of optimized two-stage endothelial differentiation protocol. Stage 1: conversion of h-iPSCs into h-MPCs mediated by the GSK-3 inhibitor CHIR99021. Stage 2: transfection of h-MPCs with modRNA encoding ETV2 and culture in chemically defined medium. A group that used modRNA:GFP served as control. (b) Conversion efficiency of h-iPSCs into VE-Cadherin+/CD31+ h-iECs measured by flow cytometry at day 4 for both the S 1-S2 (left; no electroporation, no modRNA) and S1-modETV2 (right) protocols. Groups corresponding to electroporation without modRNA and electroporation with modRNA encoding GFP served as controls for the S1-modETV2 group.

FIG. 7A-C|Comparison of cell yield and morphological changes between the standard S1-S2 and the S1-modETV2 protocols. (a) Schematic of two-stage endothelial differentiation S1-S2 and S1-modETV2 protocols. (b) Total cell number and h-iEC number on day 4 after plating 1 million h-MPCs followed by differentiation into h-iECs according to either S1-S2 or S1-modETV2 protocol. (c) Phase contrast micrographs represent time course changes in morphology along the differentiation process for S1-S2 and S1-modETV2 protocols. Scale bar, 200 μm. FIGS. 8A-B|Effect of modRNA (ETV2) concentration on h-iPSC-to-h-iEC conversion efficiency at 96 h using the S1-modETV2 differentiation protocol. (a) Titration analysis by flow cytometry for electroporation-based delivery of modRNA. (b) Titration analysis by flow cytometry for lipofection-based delivery of modRNA.

FIGS. 9A-D|Phenotypical characterization of h-iECs generated by the S1-modETV2 protocol. (a) Schematic of S1-modETV2 protocol to generate, purify, and expand h-iECs. (b-d) h-iECs were analyzed at day 7. (b) Contrast phase images of confluent h-iECs with typical cobblestone morphology. Scale bar, 200 μm. (c) mRNA expression (qRT-PCR) of endothelial markers (KDR, CDH5, PECAM1, ERG TEK, NOS3 and VWF) and pluripotency marker (OCT4). h-iPSCs and ECFCs served as controls. Data normalized to GAPDH expression. (d) Immunofluorescence staining for CD31, VE-Cadherin, UEA-1 and VWF in h-iECs generated by the S1-modETV2 protocol. Nuclei stained by DAPI. Scale bar, 100 μm.

FIG. 10|Immunofluorescence staining for VE-Cadherin and SM22 between S1-S2 and S1-modETV2 protocols at day 4. Nuclei stained by DAPI. Scale bar, 100 μm. Percentage of SM22+/VE-Cadherin-cells. Bars represent mean s.d.; n=5. ***P<0.001 between S1-S2 and S1-modETV2 protocols.

FIGS. 11A-D|Characterization of h-iPSC clones generated from different donors and tissues. (a) Schematic of generation of thirteen h-iPSC clones from dermal fibroblasts (dFB), umbilical cord blood-derived ECFCs (cbECFC), and urine-derived epithelial cells (uEP). (b) Immunofluorescence staining for pluripotency markers including OCT4, SOX2 and NANOG Nuclei stained by DAPI. Scale bar, 50 μm. (c) Teratoma formation upon implantation of h-iPSCs into nude mice for 4 weeks. Hematoxylin and eosin (H&E) staining of explanted tumors showed three germ layers including neuroepithelial rosettes (N), endodermal gut-like tissues (E) and mesenchymal stromal tissue (M). Scale bar, 100 μm. (d) Generation of EGFP-labeled h-iPSC with transposase mediated knock-in of a PiggyBac transposon plasmid. After puromycin selection and clonal expansion, homogenous green fluorescent clones of h-iPSC-GFP were generated. Scale bar, 100 μm.

FIGS. 12A-B|Time course analysis of mRNA expression (qRT-PCR) of mesodermal markers (TBXT and MIXL1), endothelial commitment transcription factors (ETV2 and ERG), endothelial markers (PECAM1, CDH5, NOS3, VWF, TEK and KDR), pluripotency marker (POU5F1) and smooth muscle marker (ACTA2) in (a) the standard S1-S2 differentiation protocol (b) the S1-modETV2 differentiation protocol. Data normalized to GAPDH expression.

FIGS. 13A-D|Generation of h-iPSCs-KDR^{-/-} and h-iPSCs-ETV2^{-/-} clones by CRISPR/Cas9. (a) Sanger sequencing of the two edited alleles encoding the 3^{rd} exon of KDR; SEQ ID NOs. 2-4 appear in order. (b) Flow cytometry showed the conversion of h-iPSCs-KDR^{-/-} into FLK1-/CD31+ h-iECs at 48 h using the early modETV2 protocol. (c) Sanger sequencing of the two edited alleles encoding the 4^{th} exon of ETV2; SEQ ID NOs. 5-7 appear in order. (d) Immunofluorescence staining for ETV2 at 72 h using the S1-52 differentiation protocol. Nuclei stained by DAPI. Scale bar, 200 μm.

FIGS. 14A-B|Effect of VEGF-A concentration on h-iEC yield using the S1-52 differentiation protocol. (a) Dose dependent conversion efficiency of h-iPSCs into CD31+ h-iECs by flow cytometry. (b) Immunofluorescence staining for ETV2 and VE-Cadherin at 72 h with different concentrations of VEGF-A. Nuclei stained by DAPI. Scale bar, 50 μm.

FIG. 15| Differences in differentiation efficiency between four alternative S1-S2 methodologies and the S1-modETV2 protocol for h-iPSC clones lacking either ETV2 and KDR (h-iPSC-ETV2^{-/-} and h-iPSC-KDR^{-/-}). Only S1-modETV2 protocol could successfully derive h-iECs from either h-iPSC-ETV2^{-/-} or h-iPSC-KDR^{-/-} cell lines with high efficiency. In contrast, the four alternative S1-S2 methodologies failed to get any h-iECs.

FIGS. 16A-F|Generation of h-iECs with the early modETV2 differentiation protocol. (a) Schematic of the early modETV2 protocol. (b) Effect of modRNA (ETV2) concentration on h-iPSC-to-h-iEC conversion efficiency at 48 h and 96 h. Titration analysis by flow cytometry for electroporation-based delivery of modRNA. (c) Flow cytometry analysis of differentiation efficiency at 48 h (early modETV2 protocol) and 96 h (S1-S2 protocol) in 13 h-iPSC clones generated from dermal fibroblasts (dFB), umbilical cord blood-derived ECFCs (cbECFC), and urine-derived epithelial cells (uEP). (d) Differences in differentiation efficiency between early modETV2 and S1-S2 protocols for all 13 h-iPSC clones. Bars represent mean s.d. (e) Time course immunofluorescence staining for ETV2, CD31 and OCT4 during the early modETV2 differentiation protocol. Nuclei stained by DAPI. Scale bar, 100 μm. Phase contrast micrographs represent time course morphological changes of cells during the early modETV2 protocol. Scale bar, 200 μm. (f) Time course analysis of mRNA expression (qRT-PCR) of mesodermal markers (TBXT and MIXL1), endothelial commitment transcription factors (ETV2 and ERG), endothelial markers (PECAM1, CDH5, NOS3, VWF, TEK and KDR), pluripotency marker (POU5F1) and smooth muscle marker (ACTA2) in h-iECs generated with the early modETV2 protocol. Data normalized to GAPDH expression.

FIGS. 17A-D|Early modETV2 protocol bypassed the intermediate mesodermal stage. (a) Schematic of S1-S2 and early modETV2 differentiation protocols. (b) Time course analysis of mRNA expression (qRT-PCR) of mesodermal markers (TBXT and MIXL1) in the S1-S2 and the early modETV2 protocols—(c) Effect of small molecule inhibitors SB431542 (10 μM), Wnt-C59 (5 μM), and SU5416 (5 μM) on the percentage of h-iECs generated with the S1-52 (96 h) and the early modETV2 (48 h) differentiation protocols. (d) Quantification on the percentage of h-iECs (CD31+) by flow cytometry for both differentiation protocols and each inhibitor.

FIGS. 18A-D|Transcriptome analysis of h-iECs obtained from various differentiation protocols. (a) Venn diagram on the number of differentially expressed genes in h-iECs and ECFCs that were upregulated compared to h-iPSCs. (b) Heatmap of the normalized expression of selected pluripotent genes across all samples. (c) GO analysis on all differentially expressed genes between h-iECs generated with the S1-modETV2 and the early modETV2 differentiation protocols. GO terms displayed corresponding to positive and negative enrichment for h-iECs generated with the S1-modETV2 protocol. (d) GO analysis on differentially expressed genes from EC clusters #5 and #10 between h-iECs generated with the S1-modETV2 and the early modETV2 differentiation protocols. GO terms displayed corresponding to the negative enrichment for h-iECs generated with the S1-modETV2 protocol.

FIGS. 19A-C|Robust endothelial phenotype of h-iECs along their expansion in culture. h-iECs generated by the S1-modETV2 protocol maintained an endothelial phenotype during in vitro expansion. h-iECs were analyzed at three different time points of expansion (namely, day 4, day 11, and day 21). (a) Flow cytometry analysis revealed that h-iECs remained fairly pure during expansion (>95% cells are VE-cadherin+/CD31+). The expanded h-iECs maintained expression of EC markers at the mRNA (b) and protein (c) levels and remained negative for POU5F1 (OCT4) and α-Smooth muscle actin (α-SMA).

FIG. 20|Differentiation of human MSCs into smooth muscle cells (SMCs) upon co-culture with h-iECs. h-iECs generated by S1-modETV2 protocol induced h-MSCs-GFP into SMCs expressing MYH11 during the co-culture assay. Double staining of MYH11 and GFP revealed that MYH11 was co-localized with GFP, confirming that those SMCs were derived from the h-MSCs-GFP. Scale bar, 100 μm.

FIGS. 21A-B|Production of nitric oxide (NO) by h-iECs. h-iECs generated by the S1-modETV2 protocol produced NO which generated a green fluorescent signal upon administration of DAF-FM. Flow cytometry analysis (a) and immunofluorescence images (b) were acquired. The presence of L-NAME (a drug that decreases NO production) served as control. Scale bar, 100 μm.

FIG. 22|Human blood vessels formed in vivo by h-iECs-GFP. Immunofluorescence staining of explanted grafts that contained h-iECs-GFP after 7 days in vivo. Human lumens stained by GFP (green). Perivascular cells stained by α-SMA (red). Nuclei stained by DAPI. Please note that the red blood cells within the lumen had auto-fluorescence. Scale bar, 50 μm.

FIGS. 23A-C|In vivo vascular network-forming ability of h-iECs without h-MSCs. (a) Grafts contained only h-iECs that were generated by the S1-modETV2 protocol. Images are macroscopic views of the explanted grafts from four mice at day 7. (b) Hematoxylin and eosin (H&E) staining of explanted grafts after 7 days in vivo. Perfused vessels were identified as luminal structures containing red blood cells (yellow arrowheads). Scale bar, 400 μm. (c) Immunofluorescent staining of explanted grafts after 7 days in vivo. Human lumens stained by h-CD31. Perivascular coverage stained by α-SMA. Nuclei stained by DAPI. Scale bar, 100 μm.

FIGS. 24A-J: Generation of HA-iPSCs and HA-iECs from hemophilia A patients. (a) A list of the 7 severe hemophilic patients with their corresponding mutant genotype from whom urine samples were taken. (b) Schematic overview of epithelial cell isolation from patient urine (HA-UECs), reprogramming to patient induced pluripotent stem cells (HA-iPSCs) through episomal expression of reprogramming factors (Oct4, Sox2, Klf4, L-Myc, Lin28), and differentiation to hemophilia patient endothelial cells (HA-iECs) using modified RNA (ETV2). (c) Phase contrast imaging of the initial appearance (left) and expansion (right) of cells during the reprogramming of HA-UECS (top) to HA-iPSCs (middle), and subsequent differentiation into HA-iECs (bottom) (scale bar 5×). (d) Characterization of highly pure HA-iPSCs through positive immunofluorescence staining of stem cell markers (OCT4, SOX2, NANOG) without endothelial marker expression (CD31), and (e) FACS analysis showing high percentages of stem cell surface marker (SSEA4, Tra-1-81) expression. (f) Teratoma-forming assay in nude mice showing the ability of HA-iPSCs to form into a large teratoma containing 3 different germ layers (labeled) in vivo after hematoxylin and eosin (H&E) staining analysis (scale bar H&E 32X). (g) Sanger sequencing data confirming individual F8 mutations in HA-iPSCs after reprogramming both for point mutation (patient #1 left) and inversion (patient #5 right). SEQ ID NOs: 8-9 appear in order. (h) FACs analysis of differentiation efficiency of HA-iPSCs into CD31+/VE-Cadherin+ HA-iECs (top right box), which was similar to the purity of differentiation (75-97%) in non-hemophilic human iPSC clones (Control-iPSCs). (i) Characterization of HA-iECs through positive immunofluorescence staining for endothelial cell markers (CD31, VE-Cadherin, and vWF) without stem cell marker (OCT4) expression. (j) Further FACs confirmation of highly efficient differentiation through high levels of endothelial surface markers (CD31, VE-Cadherin) and loss of stem cell markers (Tra-1-81, SSEA4). Nuclei were stained with DAPI. In panel h, bars represent mean±s.d.; n.s=no statistical differences.

FIGS. 25A-E: Stable expression of full-length FVIII in HA-iECs by piggyBac vectors. (a) Genetic map of (1) piggyBac transposon vector with full-length Factor 8 (FL-F8) transgene with a yellow B-Domain, and (2) super piggyBac transposase expression vector. Underneath, a diagram overview of our transfection strategy of HA-iPSCs followed by differentiation to HA-FLF8-iEC using our modRNA (ETV2) method. (b) Confirmation of full-length transgene insertion into HA-FLF8-iECs (right) through PCR of cDNA showing a presence of 3 fragments (˜245 bp DNA fragment for P1-P2 primers, ˜290 bp for P3-P4, and ˜3 kbp for P1-P4) compared to minimal bands in unedited HA-iECs (left) and a singular short fragment (˜401 bp DNA fragment for P1-P4) over the B-Domain deletion in control ECs with a BDD-F8 insert (ECFCs-BDD-F8). (c) qRT-PCR analysis confirming F8 mRNA overexpression in HA-FLF8-iPSCs and HA-FLF8-iECs after differentiation in 5 edited clones (F8-C1-5) compared to the unedited control cells (n=3). (d) Graphical outline of the linear relationship (R²=0.79) between PB insertion number (x-axis) and expression of F8 transgene (y-axis) in the same five HA-F8FL-iEC clones and unedited control. (e) Immunofluorescent co-staining of FVIII (green) and vWF (red) protein in both HA-iECs and edited HA-FLF8-iECs showing overexpression of FVIII protein. All nuclei were stained with DAPI. In panels c-d, F8 expression was normalized to 10\hat{ }3 GAPDH.

FIGS. 26A-F: Bioengineering hemophilia A patient-specific FVIII-secreting vascular networks in hemophilic mice. (a) Schematic of microvascular graft models. Grafts were prepared by combining either HA-iECs (n=5) or HA-FLF8-iECs (n=10) with h-MSCs in hydrogels followed by subcutaneous injection into a hemophilic mouse (SCID-f8ko). After 7 days, the implants were excised for analysis. Preliminary macroscopic analysis of implants suggests similarities in the degree of vascularization between control and gene-edited groups (size and redness). (b) Characteristic hematoxylin and eosin (H&E) staining of an explanted HA-FLF8-iEC implant, identifying perfused microvessels (yellow arrows) containing erythrocytes throughout the implant. (c) Comparison of microvessel density (perfused vessels/mm²) between HA-iEC and HA-F8FL-iEC implants showing similar implant vascularization. (d) Immunofluorescent staining of grafts identifying perfused human lumens (hCD31+) invested with perivascular cells (α-SMA), demonstrating genuine human blood vessels in both implant groups. These vessels also contain erythrocytes demonstrating the functional anastomoses of our xenografts with the host bloodstream. (e) Percentage of mural cell investment around the newly formed vessels (hCD31+aSMA+/CD31+) with no differences between edited and unedited groups demonstrating that PB gene-editing has no significant impact on iEC vasculogenesis or recruitment of support cells. (f) Grafts formed with HA-FLF8-iECs maintained overexpression of FVIII in human blood vessels—identified by co-expression of h-CD31 and hFVIII—while grafts formed by unedited HA-iECs had virtually undetectable levels of FVIII protein in their human vessels. All nuclei were stained with DAPI. In panel c and e, n.s.=no statistical differences.

FIGS. 27A-D: Secretion of FL-FVIII into the bloodstream and correction of coagulation deficiency in hemophilic mice. (a) To analyze the ability of our implants to secrete full length FVIII protein and restore hemostasis, a tail tip bleeding assay was performed on day 7 after injection of unedited (HA-iECs) and edited (HA-FLF8-iECs) implants into hemophilic mice (SCID-f8ko). Hemophilic and healthy SCID mice with no implants served as controls, and characteristic images of the bleeding assay for each group are shown. (b-c) Percent body weight loss, used to quantify blood loss, and bleeding time were recorded over the duration of the 20 min bleeding assay with significant lowering of bleeding time and body weight loss percentage to healthy levels in mice that received HA-FLF8-iEC implants. (d) FVIII activity levels in blood plasma collected from each mouse group after the bleeding assay with dramatically increased levels of circulating FVIII released from our HA-F8FL-iEC implant. No implant control groups n=4, HA-iECs n=5, HA-FLF8-iEC n=9-12. In b-d, bars represent mean+/−sd, n.s.=no statistical differences, **P<0.01, ***P<0.001.

FIG. 28| Expansion potential of HA-FLF8-iECs. Expansion potential in culture of two independent clones of HA-FLF8-iECs (termed clones FC2 and FC3).

FIGS. 29A-B|Characterization of edited HA-F8FL-iPSCs. (a) HA-F8FL-iPSC immunostaining positively for stem cell markers (OCT4, SOX2) and negatively for endothelial surface marker expression (CD31). (b) Top, Teratoma formation upon implantation of HA-F8FL-iPSCs into nude mice for 4 weeks. Bottom, Hematoxylin and eosin (H&E) staining of explanted tumors showed three germ layers including melanocytes of ectodermal origin (ECT), endodermal gut-like tissues (END) and mesenchymal stromal tissue (MES). Scale bar, 100 μm.

FIG. 30|Characterization of edited HA-F8FL-iECs. Immunofluorescent staining of endothelial markers (CD31, VE-cad, vWF) demonstrating efficient generation of HA-FLF8-iECs with uniform endothelial marker expression after PB editing.

FIG. 31| Graphical overview of FVIIIKO-SCID mouse generation.

### DETAILED DESCRIPTION

The advent of human induced pluripotent stem cells (h-iPSCs) created an exciting and non-invasive opportunity to obtain patient-specific ECs. However, differentiating h-iPSCs into ECs (herein referred to as h-iECs) with high efficiency, consistently, and in high abundance remains a challenge³.

Current differentiation protocols are inspired by vascular development and rely on sequentially transitioning h-iPSCs through two distinct stages (referred to as stages 1 and 2 or S1-S2)⁴. During S1, h-iPSCs differentiate into intermediate mesodermal progenitor cells (h-MPCs), a process regulated by Wnt and Nodal signaling pathways. In S2, h-MPCs acquire endothelial specification principally via VEGF signaling 4. Existing protocols, however, are far from optimal. Limitations stem from the inherent complexity associated with developmental processes. First, directing h-MPCs to solely differentiate into h-iECs is not trivial. Indeed, recent reports estimate that with the canonical S1-S2 approach, less than 10% of the differentiated cells may actually be bona fide h-iECs³. In addition, achieving consistent differentiation in different h-iPSC lines continues to be a challenge⁵. This dependency on cellular origin makes the clinical translation of h-iECs problematic.

Herein described is the development of a protocol that enables more consistent and highly efficient differentiation of human h-iPSCs into h-iECs. The results showed that a critical source of inconsistency resided in the inefficient activation of the transcription factor E26 transformation-specific (ETS) variant 2 (ETV2) during S2. To circumvent this constraint, a chemically modified mRNA (modRNA) was used; in recent years this technology has improved the stability of synthetic RNA allowing its transfer into cells (and subsequent protein expression) in vitro and in vivo⁶. A synthetic modRNA was developed to uniformly activate ETV2 expression in h-MPCs, independently of VEGF signaling.

The present protocol entails a total differentiation period of about 4 days and comprises two steps: 1) differentiation of h-iPSCs into intermediate h-MPCs; and 2) conversion of h-MPCs into h-iECs upon delivery of modRNA encoding ETV2. This S1-modETV2 approach allowed widespread expression of ETV2 throughout the entire h-MPC population, thus overcoming one of the main hurdles of current protocols. Using this customized protocol, 13 different human h-iPSC clonal lines were reproducibly and efficiently (>90%) differentiated into h-iECs. Using these methods, h-iECs were produced at exceedingly high purity irrespective of the h-iPSC donor and cellular origin, and there were no statistical differences in efficiency. Of note, this high efficiency and reproducibility were absent when the standard S1-S2 protocol, which relies on VEGF signaling for endogenous ETV2 activation, was used. In addition, the resulting h-iECs could be expanded with ease, obtaining an average h-iEC-to-h-iPSC ratio of ˜70-fold after 3 weeks in culture. More importantly, the h-iECs were phenotypically, transcriptionally, and functionally consistent with bona fide ECs, including a robust ability to form perfused vascular networks in vivo.

Over the last decade, refinements to the standard S1-S2 differentiation protocol have steadily improved efficiency. Improvements have included, for example, the inhibition of the Notch and the TGF-β signaling pathways, the activation of protein kinase A or the synergistic effects of VEGF and BMP4 during S^{2,7,8,18}. However, most of these advances have been largely incremental, and consensus holds that the differentiation of h-iPSCs into h-iECs remains somewhat inconsistent¹⁹. The incorporation of BMP4 during S2 was shown to produce a significant improvement in differentiation efficiency; however, the mechanism behind this improvement remains unknown and thus it is unclear whether this approach can consistently produce high efficiency across multiple clonal iPSC lines, independently of their cellular origin⁷. One of the major difficulties is related to the necessary transition through the intermediate h-MPCs, which serve as common progenitors to not only h-iECs but also to other end-stage mesodermal cell types^{20,21}. Thus, directing h-MPCs to solely differentiate into h-iECs is a challenge. Indeed, a recent study that used single-cell RNA analysis revealed that after S1-52, non-endothelial cell populations (including, cardiomyocytes and vascular smooth muscle cells) were in fact predominant among the differentiated cells, and less than 10% were actually identified as bona fide ECs³. Studies have also shown that EC specification is dictated by a transient activation of ETV2, which in turn depends on VEGF signaling^{11,22}. However, our study has revealed that the activation of endogenous ETV2 during S2 is inherently inefficient and increasing the concentration of VEGF can only improve this constraint to a certain degree. This limited ability of exogenous VEGF to enhance efficiency could be explained by the fact that VEGF has also been shown to promote h-iPSC differentiation into other mesodermal fates, including cardiac progenitor cells, cardiomyocytes and hepatic-like cells^{3,20,23-25}. Thus, in order to improve efficiency, VEGF activation of ETV2 must be accompanied by inhibition of all other competing fates, which is not trivial. The present approach, however, circumvents this challenge by transiently expressing ETV2, e.g., using modRNA, in a high percentage of h-MPCs and independently of VEGF signaling. This, in turn, allowed widespread conversion into h-iECs thereby eliminating the problem of inefficiency.

Current protocols are also limited by inconsistent results among different h-iPSC lines. Indeed, a recent study examined genetically identical h-iPSC clonal lines that were derived from various tissues of the same donor and found that by following the standard S1-S2 protocol, both differentiation efficiency and gene expression of the resulting h-iECs varied significantly depending on the source of h-iPSCs⁵. The present study also found inconsistencies in differentiation efficiency between h-iPSC clonal lines with different cellular origins, including lines with identical genetic make-up and lines derived from the same tissues in different donors. This lack of consistency is certainly undesirable from a clinical translation standpoint¹⁴. Also, dependency on cellular origin may explain why published results on differentiation efficiency are often mixed and rely on selecting h-iPSC clones that are particularly attuned to EC differentiation. The present method eliminated this uncertainty and consistently produced high efficiency differentiation, irrespective of the donor and cellular origin from which the h-iPSC clones are derived.

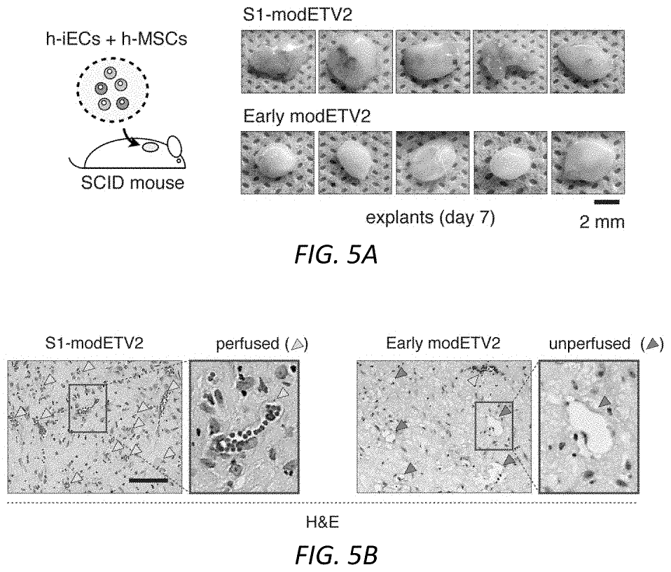

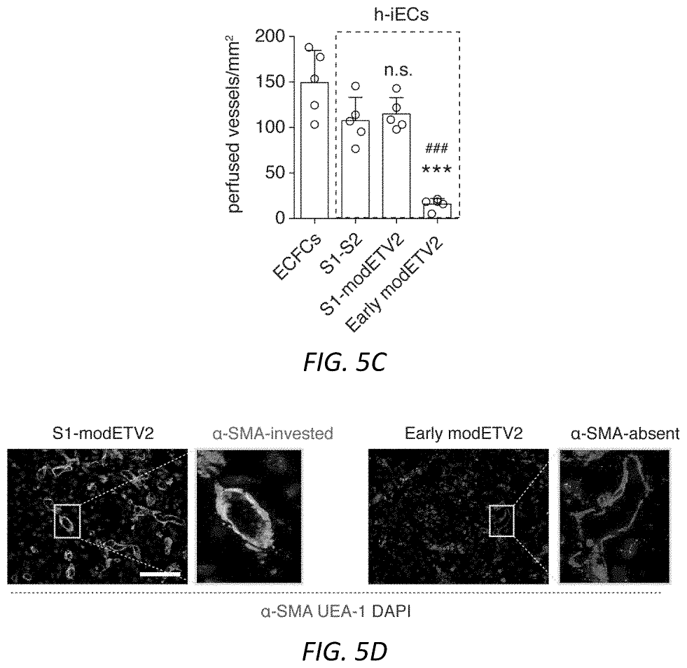

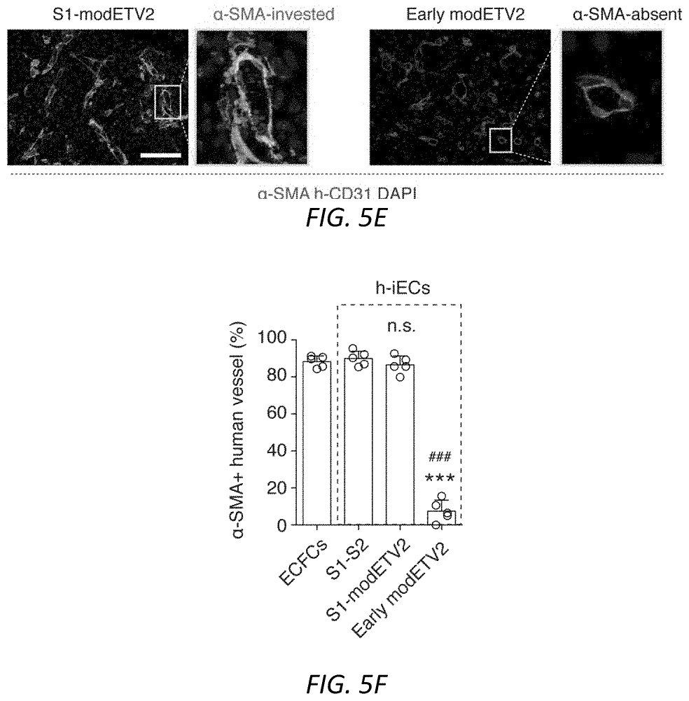

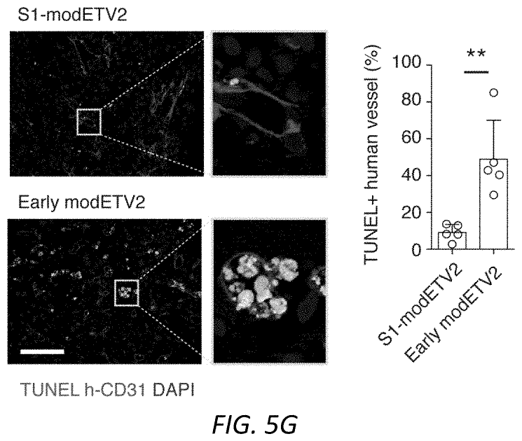

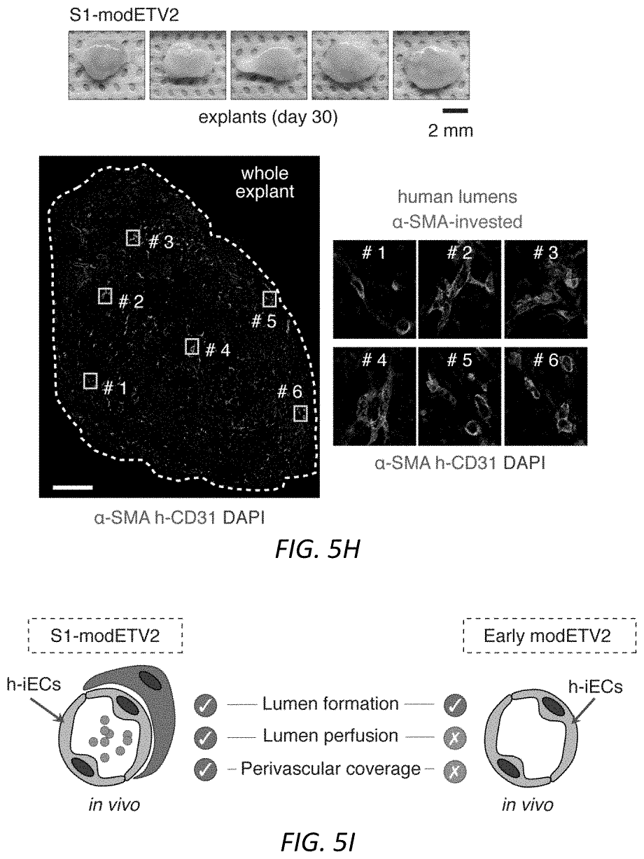

Previous studies have shown that ETV2 plays a non-redundant and indispensable role in vascular cell development^{10,26-28}. In addition, expression of ETV2 is only required transiently, ideal for non-integrating transfection strategies such as those based on modRNA⁶. Recent studies have proposed reprogramming somatic cells using transducible vectors encoding ETV2²⁹⁻³². Nevertheless, the efficiency of direct reprogramming somatic cells into ECs remains exceedingly low and achieving proper EC maturation requires long periods of time in culture. Alternatively, a few studies have recently introduced the idea of inducing ETV2 expression directly on h-iPSCs^{12,13,33}. However, to date, methods have relied on early activation of ETV2 in the h-iPSCs, thus bypassing transition through an intermediate mesodermal stage. Also, the functional competence of the resulting h-iECs remains somewhat unclear. In this regard, our study provides an important new insight: that timely activation of ETV2 is critical, and bypassing the intermediate mesodermal stage is detrimental. Indeed, h-iECs generated by our S1-modETV2 methodology displayed proper blood vessel-forming ability in vivo, whereas putative h-iECs generated by the early modETV2 approach displayed impaired functionality and were unable to robustly form perfused vessels with adequate perivascular stability (FIG. 5i).

In summary, described herein is a protocol that enables highly efficient and reliable differentiation of human h-iPSCs into competent h-iECs. The protocol is simple, rapid, and entails transient expression of the transcription factor ETV2, e.g., by delivery of modified mRNA encoding ETV2, at the intermediate mesodermal stage of differentiation. This protocol has broad application in regenerative medicine because it provides a reliable means to obtain autologous h-iECs for vascular therapies.

Methods of Generating Endothelial Cells from iPSC

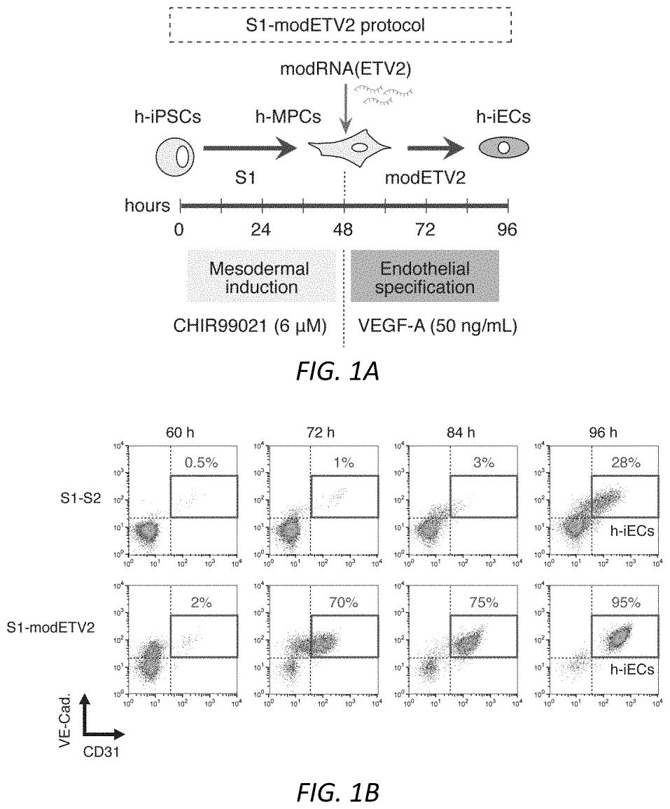

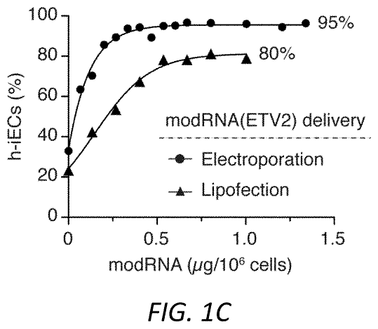

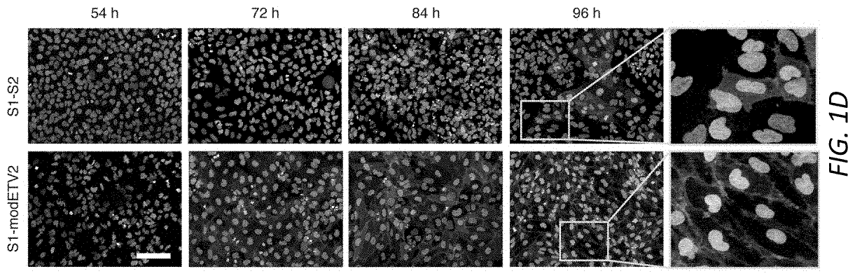

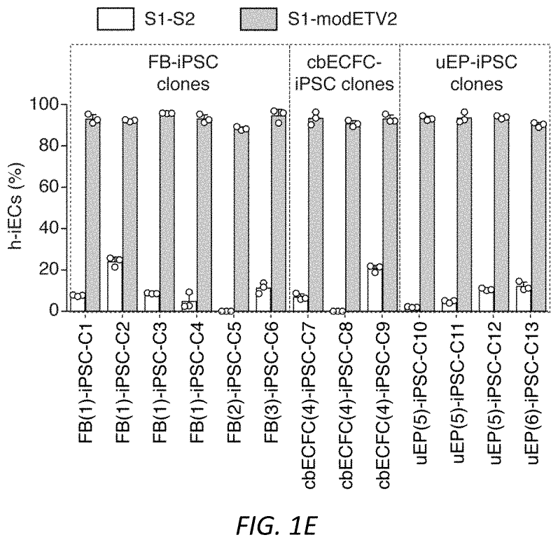

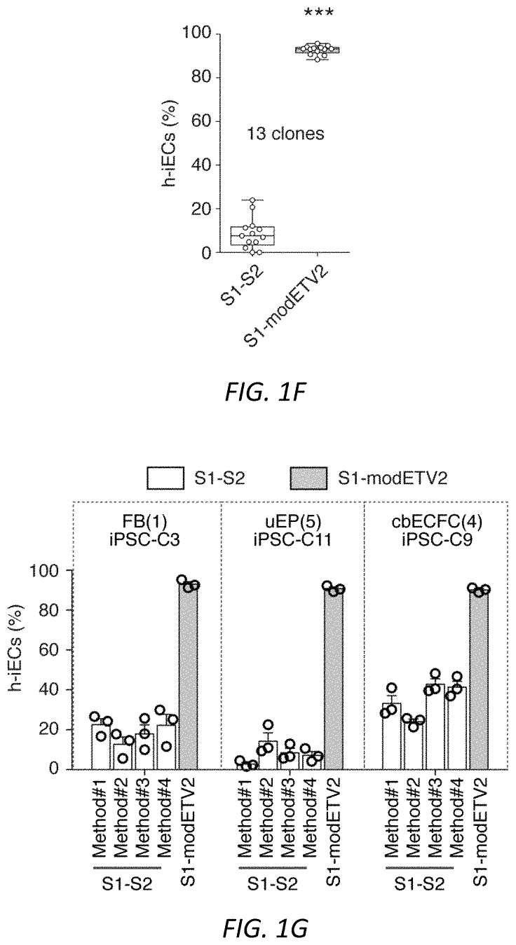

Described herein are two-dimensional, feeder-free, and chemically defined protocols that can be used to generate endothelial cells from pluripotent stem cells. The methods transition h-iPSCs through two distinct stages, each lasting about 48 hours. First is the conversion of h-iPSCs into h-MPCs. This step, similar to that in the standard S1-52 differentiation protocol, is mediated by the activation of Wnt and Nodal signaling pathways, e.g., using a glycogen synthase kinase 3 (GSK-3) inhibitor, e.g., CHIR99021 (FIG. 1a). Second, the h-MPCs are converted into h-iECs. In the present protocol, this second step is substantially different from the standard S1-52 protocol, which relies on activation of endogenous ETV2 via VEGF signaling. In contrast, the present protocol includes transiently expressing exogenous ETV2, e.g., using chemically modified RNA (modRNA) to deliver exogenous ETV2 to h-MPCs via either electroporation or lipofection (FIG. 1a).

iPSC

The methods described herein can include the use of induced pluripotent stem cells (iPSCs) that can be generated using methods known in the art or described herein. In some embodiments, the methods for generating iPSC can include obtaining a population of primary somatic cells from a subject. Preferably the subject is a mammal, e.g., a human. In some embodiments, the somatic cells are fibroblasts. Fibroblasts can be obtained from connective tissue in the mammalian body, e.g., from the skin, e.g., skin from the eyelid, back of the ear, a scar (e.g., an abdominal cesarean scar), or the groin (see, e.g., Fernandes et al., Cytotechnology. 2016 March; 68(2): 223-228). Other sources of somatic cells for hiPSC include hair keratinocytes (Raab et al., Stem Cells Int. 2014; 2014:768391), blood cells, or bone marrow mesenchymal stem cells (MSCs) (Streckfuss-Bomeke et al., Eur Heart J. 2013 September; 34(33):2618-29). In some embodiments, the cells are obtained from urine. (See, e.g., Zhou et al., Nature Protocols 7: 2080-2089 (2012).

The somatic cells are then subject to dedifferentiation protocols, e.g., using the so-called Yamanaka factors, i.e., Oct4, Sox2, Klf4,and L-Myc, orh-oct4, h-sox2, h-klf4, h-myc, h-lin-28(proteinlin-28 homolog A) and EBNA-1(Epstein-Barr Nuclear Antigen-)(see, e.g., the methods below and Takahashi, K. & Yamanaka, S. Nat Rev Mol Cell Biol 17, 183-193 (2016); Tanabe, K., Nakamura, M., Narita, M., Takahashi, K. &Yamanaka, S. Proc Natl Acad Sci US A110, 12172-12179 (2013); Nakagawa, et al., Proc Natl Acad Sci US A107, 14152-14157 (2010); Takahashi, K. &Yamanaka, S. Cell 126, 663-676 (2006)).

References to exemplary sequences for OCT4, KLF4, SOX2, L-MYC, Lin-28 and EBNA-1 are provided in the following table.

| Gene | Nucleic acid → protein | |
|---|---|---|
| OCT4 | NM_002701.6 → NP_ 002692.2 | Isoform 1 |
| (POU class 5 | NM_001173531.2 → NP_001167002.1 | Isoform 2 |
| homeobox 1 | NM_001285987.1 → NP_001272916.1 | Isoform 3 |
| (POU5F1)) | NM_001285986.1 → NP_001272915.1 | Isoform 4 |
| KLF4 | NM_001314052.1 → NP_001300981.1 | Isoform 1 |
| (Kruppel like | NM_004235.6 → NP_004226.3 | Isoform 2 |
| factor 4) | | |
| SOX2 | NM_003106.4 → NP_ 003097.1 | |
| (SRY-box 2) | | |
| L-MYC | NM_001033081.3 → NP_001028253.1 | Isoform 1 |
| (MYCL | NM_005376.4 → NP_005367.2 | Isoform 2 |
| proto- | NM_001033082.2 → *NP_001028254.2 | Isoform 3 |
| oncogene, | | |
| bHLH | | |
| transcription | | |
| factor) | | |
| EBNA-1 | NC_007605.1 (95662-97587) → YP_401677.1 | |
| Lin-28 | NM_024674.6 → NP_078950.1 | |

The presence of iPSCs can be confirmed, e.g., by expression of pluripotent transcription factors OCT4, NANOG, and SOX2 and/or by the ability to form teratomas, e.g., using a teratoma formation assay as known in the art. Once obtained, the iPSC can be maintained in culture using standard methods, e.g., iPSC culture media such as mTeSR1, mTeSR-E7, optionally in the presence of a rho-associated protein kinase (ROCK) inhibitor, e.g., Y27632, GSK429286A, Y-30141, Fasudil, Ripasudil, or Netarsudil. The ROCK inhibitors promote the survival of dissociated iPS cells, and improve the clonal growth of iPS cells.

The iPSC can be made from cells from any species, e.g., any mammalian species, but are preferably human. As noted above, as an alternative to iPSC, hESC can also be used.

Stage 1—Conversion of iPSCs into MPCs

The first stage of the present methods is the conversion of iPSCs (or alternatively Embryonic stem cells (h-ESCs)) into MPCs. The methods include incubating the cells in media in the presence of a GSK3 inhibitor, e.g., CHIR99021, BIO, NP31112, IM-12; pyrazolopyrimidine derivatives, Benzimidazoles (e.g., analogs of 7-hydroxy-1H-benzimidazole), Pyridinones (e.g., 4-(4-hydroxy-3-methylphenyl)-6-phenyl pyrimidin-2-ol), Pyrimidines, Indolylmaleimide analogs, Imidazopyridines, Oxadiazoles, Pyrazines, thiadiazolidinones, Emodin and 4-Aminoethylamino Emodin, and 5-Imino-1,2,4-Thiadiazoles (ITDZs). See, e.g. Pandey and DeGrado, Theranostics. 2016; 6(4): 571-593.

Other optional ingredients include ascorbic acid, to promote the differentiation of iPS cells into mesodermal intermediates (MPCs), and L-glutamine, a nutrient in cell cultures for energy production as well as protein and nucleic acid synthesis. GlutaMAX is an improved cell culture supplement that can be used as a direct substitute for L-glutamine in cell culture media. As long as the GSK3 inhibitor is included, the other components can be reformulated.

The cells are incubated in stage 1 for about 48 hours (i.e., 48±12, 10, 8, 6, 4, or 2 hours), until the cells express mesodermal markers, e.g., TBXT (also known as brachyury at the protein level), MIXL1, and KDR (VEGFR2). In some embodiments, brachyury staining is used.

Stage 2—Conversion of MPCs to iECs

In the second stage, the MPCs are converted into iECs. The MPCs are preferably dissociated into single cells, and then induced to transiently express exogenous ETV2. During stage 2, the cells can optionally incubated in media comprising one or more growth factors, including VEGF-A, FGF-2, and EGF, and a TGFbeta receptor antagonist, e.g., SB431542, dihydropyrrolopyrazoles (e.g., LY550410 and LY580276); imidazoles (e.g., SB-505124 from GlaxoSmithKline), pyrazolopyridines, pyrazoles, imidazopyridines, triazoles, pyridopyrimidines, and isothiazoles. Specific examples include galunisertib (LY2157299 Monohydrate); A 83-01; RepSox; SD 208; SB 505124; LY 364947; D 4476; SB 525334; GW 788388; R 268712; IN 1130; SM 16; A 77-01; and SB431542. See, e.g., de Gramont, Oncoimmunology. 2017; 6(1): e1257453. As long as ETV2 expression is induced, media for the 2 days of S2 just needs to be formulated to keep cells healthy. These components are certainly helpful, and were used in the exemplary methods below to promote endothelial cell growth; but they are not essential for differentiation of MPCs into iECs in the present protocols. After the about 48 hours of stage 2, then media should be re-formulated for endothelial cell growth.

The cells are incubated in stage 2 for about 48 hours (i.e., 48±48±12, 10, 8, 6, 4, or 2 hours) or more (one can keep culturing cells in S2 media without having to transition into a third stage), at least until the cells have cobblestone-like morphology; express endothelial cell markers at the mRNA and (more preferably) protein levels, e.g., CD31 and/or VE-Cadherin (e.g., using flow cytometry); do not express the pluripotent marker OCT4; and/or bind Ulex europaeus agglutinin I (UEA-1).

Once differentiation to iECs is achieved, the cells can be maintained in culture, expanded, genetically modified, frozen, and/or administered to a subject.

Transient expression of ETV2

Transient expression of ETV2 can be accomplished using means known in the art, e.g., transfection with a nucleic acid encoding an ETV2 sequence, e.g., naked DNA, RNA, or a vector, e.g., a plasmid or viral vector, comprising a sequence encoding ETV2. Preferably, the nucleic acid does not persist in the cells, thus providing time-limited expression of the ETV2. In preferred embodiments, expression of ETV2 in the cells is achieved using chemically modified RNA (modRNA) to deliver the exogenous ETV2 coding sequences. In some embodiments, the ETV2 encoding sequences are linked to a regulatory sequence, e.g., a promoter, that causes expression of the ETV2 in the cells. The ETV2 nucleic acids can be delivered to the MPCs using methods known in the art, including calcium phosphate or calcium chloride precipitation, DEAE-dextrin-mediated transfection, electroporation or lipofection.

In preferred embodiments, synthetic, chemically modified mRNA, wherein at least one pseudouridine is substituted for uridine and/or at least one 5-methyl-cytosine is substituted for cytosine, is used to express the ETV2 proteins. See, e.g., Karikó et al., Immunity. 2005 August; 23(2):165-75; Karikó et al., Mol. Ther. 2008. 16, 1833-1840; Karikó et al., Nucleic Acids Res. 2011 November; 39(21):e142. Warren et al. Cell Stem Cell. 2010 Nov. 5; 7(5):618-30; Lui et al. Cell Res. 2013 October; 23(10):1172-86; Zangi et al., Nat Biotechnol. 2013 October; 31(10):898-9071; Lui et al., Cell Res. 2013 October; 23(10):1172-86; and Chien et al., Cold Spring Harb Perspect Med. 2015 January; 5(1): a014035.

The ETV2 sequences used in the present methods and compositions can be at least 60%, 65%, 70%, 75%, 80%, 85%, 90%, 95%, 96%, 97%, 98%, 99% identical to the full length wild type genomic or cDNA ETV2 sequence, respectively. In some embodiments, a suitable ETV2 gene encodes a protein sequence that is at least 60%, 65%, 70%, 75%, 80%, 85%, 90%, 95%, 96%, 97%, 98%, 99% identical to a full-length wild type ETV2 protein sequence, e.g., SEQ ID NO:1 (NP_055024.2). Exemplary wild type genomic, cDNA, and protein sequences of human ETV2 are provided herein. Sequences for use in other species are known in the art.

### Exemplary Human ETV2 Sequences

| Nucleic acid | Protein | Variant/isoform |
|---|---|---|
| NM_014209.4 | NP_055024.2 | isoform 1 |
| NM_001300974.1 | NP_001287903.1 | isoform 2 |
| NM_001304549.1 | NP_001291478.1 | isoform 3 |

Variant 1 represents the longer transcript and encodes the longer isoform 1. Variant 2 differs in the 5′ UTR, lacks a portion of the 5′ coding region, and initiates translation at a downstream start codon, compared to variant 1, and encodes isoform 2, which has a shorter N-terminus, compared to isoform 1. Variant 3 lacks two in-frame exons compared to variant 1. It encodes isoform 3, which is shorter than isoform 1. In some embodiments, Variant 1/isoform 1 is used.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90% or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. In another embodiment, the percent identity of two amino acid sequences can be assessed as a function of the conservation of amino acid residues within the same family of amino acids (e.g., positive charge, negative charge, polar and uncharged, hydrophobic) at corresponding positions in both amino acid sequences (e.g., the presence of an alanine residue in place of a valine residue at a specific position in both sequences shows a high level of conservation, but the presence of an arginine residue in place of an aspartate residue at a specific position in both sequences shows a low level of conservation). For example, the percent identity between two amino acid sequences can be determined using the Needleman and Wunsch ((1970) J. Mol. Biol. 48:444-453) algorithm which has been incorporated into the GAP program in the GCG software package, using a Blossum scoring matrix, e.g., with default values for gap penalty, gap extend penalty of 4, and frameshift gap penalty.

Methods of Use—Cell Therapy

iECs generated using a method described herein can be used for cell therapy, e.g., to treat various conditions in subjects, e.g., mammalian subjects, e.g., humans or non-human veterinary subjects such as dogs, cats, horses, pigs, sheep, cows, goats, or zoo animals. As one example, the cells can be used in vascular cell therapy, e.g., to treat ischemic or vascular injury and endothelial denudation, e.g., in limbs, retina or myocardium; or for revascularization/neovascularization, e.g., to treat diabetes or promote success after organ transplantation. See, e.g., Mund et al., Cytotherapy (2009) 11(2):103-113; Rafii and Lyden, Nat Med. 2003 June; 9(6):702-12; Reed et al., Br J Clin Pharmacol. 2013 April; 75(4):897-906. These methods can include, e.g., identifying a subject in need of such treatment, and administering to the subject a population of iECs obtained using a method described herein. In preferred embodiments, the iECs are generated from iPSCs derived from the subject's own cells, i.e., are autologous.

The cells can be genetically engineered to express a heterologous, endogenous or exogenous nucleotide sequence that encodes a therapeutic polypeptide. The sequence encoding the selected protein can be inserted in an expression vector, to make an expression construct. A number of suitable vectors are known in the art, e.g., viral vectors including recombinant retroviruses, adenovirus, adeno-associated virus, herpes simplex virus 1, adenovirus-derived vectors; or recombinant bacterial or eukaryotic plasmids; or transposons, e.g., piggyBac or Sleeping Beauty (see, e.g., Tipanee et al., Hum Gene Ther. 2017 November; 28(11):1087-1104; Zhao et al., Transl Lung Cancer Res. 2016 February; 5(1):120-5). For example, the expression construct can include: a coding region; a promoter sequence, e.g., a promoter sequence that restricts expression to a selected cell type, a conditional promoter, or a strong general promoter; an enhancer sequence; untranslated regulatory sequences, e.g., a 5′untranslated region (UTR), a 3′UTR; a polyadenylation site; and/or an insulator sequence. Such sequences are known in the art, and the skilled artisan would be able to select suitable sequences. See, e.g., Current Protocols in Molecular Biology, Ausubel, F. M. et al. (eds.) Greene Publishing Associates, (1989), Sections 9.10-9.14; Vancura (ed.), Transcriptional Regulation: Methods and Protocols (Methods in Molecular Biology (Book 809)) Humana Press; 2012 edition (2011) and other standard laboratory manuals. The nucleotide sequence can include one or more of a promoter sequence, e.g., a promoter sequence; an enhancer sequence, e.g., 5′ untranslated region (UTR) or a 3′ UTR; a polyadenylation site; an insulator sequence; or another sequence that increases the expression of an endogenous peptide or increases expression, level, or activity of an endogenous polypeptide.

The iECs can be transfected directly, or can be cultured first, removed from the culture plate and resuspended before transfection is carried out. The cells can be combined with the nucleotide sequence that encodes a therapeutic polypeptide, e.g., stably integrate into their genomes, and treated in order to accomplish transfection. As used herein, the term “transfection” includes a variety of techniques for introducing an exogenous nucleic acid into a cell including calcium phosphate or calcium chloride precipitation, microinjection, DEAE-dextrin-mediated transfection, lipofection, electroporation or genome-editing using zinc-finger nucleases, transcription activator-like effector nuclease or the CRIPSR-Cas system, all of which are routine in the art (Kim et al (2010) Anal Bioanal Chem 397(8): 3173-3178; Hockemeyer et al. (2011) Nat. Biotechnol. 29:731-734; Feng, Z et al. (2013) Cell Res 23(10): 1229-1232; Jinek, M. et al. (2013) eLife 2:e00471; Wang et al (2013) Cell. 153(4): 910-918); Lin et al., “Vascular Stem Cell Therapy,” in Stem Cells and Cell Therapy, 2014 (pp. 49-69), DOI: 10.1007/978-94-007-7196-3_3.

Transfected cells can be allowed to undergo sufficient numbers of doubling to produce either a clonal cell strain or a heterogeneous cell strain of sufficient size to provide the therapeutic protein to an individual in effective amounts. The number of required cells in a transfected clonal heterogeneous cell strain is variable and depends on a variety of factors, including but not limited to, the use of the transfected cells, the functional level of the exogenous DNA in the transfected cells, the site of implantation of the transfected cells (for example, the number of cells that can be used is limited by the anatomical site of implantation), and the age, surface area, and clinical condition of the patient. The genetically modified iECs cells, e.g., cells produced as described herein, can be introduced into an individual to whom the product is to be delivered. Various routes of administration and various sites (e.g., renal sub capsular, subcutaneous, central nervous system (including intrathecal), intravascular, intrahepatic, intrasplanchnic, intraperitoneal (including intraomental), intramuscularly implantation) can be used; in general terms, the iECs can be injected into any vascularized tissue (i.e., tissue with blood vessels) so the new iECs can make capillaries that hook up with the existing vessels. Once implanted in an individual, the transfected cells produce the product encoded by the heterologous nucleic acid or are affected by the heterologous nucleic acid itself.

Hemophilia A

Hemophilia A is an inherited X-chromosome-linked bleeding disorder caused by mutations in the F8 gene encoding coagulation factor VIII (FVIII) (Gitschier, J. et al. (1985)). Hemophilia A has an incidence of 1 in 5,000 liveborn males and patients with severe hemophilia A (˜60% of all hemophiliacs) present frequent spontaneous bleeds into joints and soft tissues (hemarthrosis), which can lead to serious complications and even death (Soucie et al., 2000). Current treatments for Hemophilia A patients are infusions of FVIII concentrates (Gouw et al., 2013). However, patients require repeated intravenous injections of the factor throughout life, which creates continuous discomfort, augments morbidity, and impairs overall quality of life (Barr et al., 2002; von Mackensen et al., 2012). Moreover, prophylaxis for severe patients involves injections of FVIII concentrates every other day and adherence is a constant challenge (Lindvall et al., 2006; Walsh and Valentino, 2009). Therefore, hemophilia A remains an appealing target disease for the application of gene therapy (High, 2012; Matrai et al., 2010).

Most preclinical studies of hemophilia A gene therapy have focused on the use of viral vectors, including adenovirus (Hu et al., 2011; Brown et al., 2004) and adeno-associated virus (AAV) (Sarkar et al., 2004; Jiang et al., 2006; Lu et al., 2008). However, F8 is a relatively large gene (˜7.0 kb cDNA) and thus it cannot be effectively packaged into most existing viral vectors (High, 2012). Consequently, most efforts in hemophilia A gene therapy have been conducted with a truncated version of FVIII that lacks the B-domain (referred to as BDD-FVIII)(Miao et al., Blood. 2004 May 1; 103(9):3412-9).

Nevertheless, mounting evidence indicates that although the B-domain is not essential for coagulation, it is involved in multiple critical post-translational functions, including FVIII secretion into the bloodstreams and its later clearance from plasma (Pipe, 2009). Thus, the interest for a full-length version of FVIII (FL-FVIII) that is applicable to gene therapy remains.

Herein described is an ex vivo gene therapy approach that uses hemophilia A patients' cells to deliver full-length FVIII into the bloodstream of hemophilic subjects. In brief, patient-specific induced pluripotent stem cells (HA-iPSCs) were generated from epithelial cells isolated from severe hemophilia A patients' urine samples, and a non-viral piggyBac DNA transposon vector (PB) was used to deliver F8 into these patients' iPSCs. Of note, PBs have a large cargo size (˜9.1 kb) and thus were able to deliver full-length F8 with reasonable integration efficiency. The full-length F8 gene edited HA-iPSCs (HA-F8FL-iPSCs) were then differentiated into competent FVIII-secreting endothelial cells (HA-FLF8-iECs) with high efficiency. These genetically modified HA-FLF8-iECs were combined in a collagen hydrogel and subcutaneously injected into immunodeficient hemophilic (SCID-f8ko) mice. Following implantation, HA-FLF8-iECs self-assembled into vascular networks and the newly-formed microvessels had the capacity to deliver FVIII directly into the bloodstream of the mice, effectively correcting the clotting deficiency from an excisable subcutaneous implant. Collectively, these studies established the feasibility of using implants containing drug-secreting vascular networks as a novel autologous ex vivo gene therapy approach to treat hemophilia A.

Thus, provided herein are methods for treating an individual who suffers from a blood clotting disorder (e.g., hemophilia A or hemophilia B) by implantation of cells producing a compound described herein, e.g., a functional factor VIII polypeptide for hemophilia A or a functional factor IX polypeptide for hemophilia B, as described herein.

The following table shows exemplary sequences for factors VIII and IX.

| Nucleic acid | Protein | Variant/isoform |
|---|---|---|
| NM_000132.3 | NP_000123.1 | coagulation factor VIII isoform a |
| | | preproprotein |
| NM_019863.2 | NP_063916.1 | coagulation factor VIII isoform b |
| NM_000133.3 | NP_000124.1 | coagulation factor IX isoform 1 |
| | | preproprotein |
| NM_001313913.1 | NP_001300842.1 | coagulation factor IX isoform 2 |
| | | precursor |

Factor VIII variant 1 of has 26 exons and encodes the full-length isoform a, while variant 2 contains an unique 5′ exon located within intron 22 of transcript variant 1 that codes for eight amino acids and is spliced to exons 23-26 maintaining the reading frame. Isoform b is considerably shorter compared to isoform a, and includes the phospholipid binding domain. Factor VIII has a domain structure of A1-A2-B-A3-C1-C2; deletion of the B domain (the BDD form, for B domain deleted) produces a form that has improved secretion (Miao et al., Blood. 2004 May 1; 103(9):3412-9).

Factor IX variant 1 is the longer transcript and encodes the longer isoform 1; variant 2 lacks an alternate in-frame exon in the 5′ coding region, and encodes isoform 2, which is shorter than isoform 1.

Any of the above sequences, or variants thereof that are at least 80%, 85%, 90%, 95%, 97%, 98%, 99%, or 100% identical to the above sequences and have the same or substantially the same clotting activity (e.g., at least 50%, 60% 70%, 80%, 90%, 95% or more of the clotting activity, e.g., as measured in a clotting assay such as the 1-stage aPTT clotting assay or 2-stage assay using the COAMATIC chromogenic assay) can be used in the present methods and compositions.

In some embodiments, the bioengineered cells are delivered inside a hydrogel-based implant; in some embodiments, the implant is placed subcutaneously and in some embodiments remains easily accessible and thus could be retrievable. A number of hydrogels are known in the art for cell implantation; for example, natural hydrogels can be made using proteins (e.g., collagen, gelatin, fibrin, or fibronectin (Fn)) or polysaccharides (e.g., hyaluronic acid (HA), agarose, alginate, chitosan, or HA-methyl cellulose (HAMC)) and combinations thereof (e.g., collagen/Ha hybrid polymers, gelatin/chitosan and fibrin/alginate polymers); synthetic hydrogels can be made using polydimethylsiloxane (PDMS), polyethylene glycol (PEG), poly(lactic-co-glycolic acid) (PLGA), polyglycerol sebacate (PGS), and Poly(propylene fumarate-co-ethylene glycol) (p(PF-co-EG), and self-assembling peptide hydrogels (self-complementary peptides (SCP) and peptide amphiphiles (PAs)), and combinations thereof, as well as natural/synthetic hybrids, e.g., PEGylated fibrinogen, elatin electrospun together with poly(L-lactic acid), hydrazide-modified gelatin with aldehyde-modified HA, and Gelatin methacrylate (GelMA). See, e.g., Liu et al., Int J Mol Sci. 2015 July; 16(7): 15997-16016; and El-Sherbiny and Yacoub, Glob Cardiol Sci Pract. 2013; 2013(3): 316-342. In some embodiments, a collagen/fibrin hydrogel or enzymatically crosslinked collagen hydrogel derived from dermal extracellular matrix. is used. See, e.g., Allen et al., J Tissue Eng Regen Med. 2011 April; 5(4):e74-86; Kuo et al., Acta Biomater. 2015 November; 27:151-166; Lin et al., Proc Natl Acad Sci USA. 2014 Jul. 15; 111(28):10137-42.

Compositions

Compositions comprising the iECS generated using a method described herein, e.g., genetically engineered iECs, and a carrier, optionally a hydrogel, are also provided herein. In some embodiments, the compositions also comprise other cell types in combination with the iECs, for example beta-cells+iECs for treating type 1 diabetes; cardiomyocytes+iECs for myocardial repair; and mesenchymal stem cells+iECs for bone regeneration.

### EXAMPLES

The invention is further described in the following examples, which do not limit the scope of the invention described in the claims.

Methods

The following materials and methods were used in the Examples below.

Isolation and Culture of Human MSCs, ECFCs and uEPs

Human MSCs (h-MSCs) were isolated from the white adipose tissue as previously described (Lin, R.-Z. et al. Proc Natl Acad Sci USA 111, 10137-10142 (2014)). h-MSCs were cultured on uncoated plates using MSC-medium: MSCGM (Lonza, Cat No. PT-3001) supplemented with 10% GenClone FBS (Genesee, Cat No. 25-514), 1×penicillin-streptomycin-glutamine (PSG, ThermoFisher, Cat No. 10378106). All experiments were carried out with h-MSCs between passage 6-10. Human ECFCs were isolated from umbilical cord blood samples in accordance with an Institutional Review Board-approved protocol as previously described (Melero-Martin, J. M. et al. Blood 109, 4761-4768 (2007)). ECFCs were cultured on 1% gelatin-coated plates using ECFC-medium: EGM-2 (except for hydrocortisone; PromoCell, Cat No. C22111) supplemented with 10% FBS, 1×PSG. All experiments were carried out with ECFCs between passage 6-8. Human urine-derived epithelial cells (uEPs) were isolated from urine samples and were cultured on 1% gelatin-coated plates using ECFC-medium. All experiments were carried out with uEPs up to passage 4.

Generation and Culture of Human iPSCs

Human induced pluripotent stem cells (h-iPSCs) were generated via non-integrating episomal transferring of selected reprogramming factors (Oct4, Sox2, Klf4, L-Myc, Lin28). Briefly, four plasmids encoding h-oct4, h-sox2, h-klf4, h-myc, h-lin-28 and EBNA-1 (Addgene plasmids #27077, #27078, #27080, and #37624 deposited by Shinya Yamanaka) were introduced via electroporation into h-MSCs, ECFCs and uEPs. Transfected cells were then cultured with mTeSR-E7 medium (STEMCELL, Cat No. 05910). H-iPSC colonies spontaneously emerged between days 15-25. Colonies were then picked and transferred to a Matrigel-coated (Corning, Cat No. 354277), feeder-free culture plate for expansion and were routinely checked for absence of mycoplasma. H-iPSCs were cultured in mTeSR1 medium (STEMCELL, Cat No. 85850) on 6-well plates coated with Matrigel. At 80% confluency, h-iPSCs were detached using TrypLE select (ThermoFisher, Cat No. 12563-029) and split at a 1:6 ratio. Culture media were changed daily. h-iPSCs phenotype was validated by expression of pluripotent transcription factors OCT4, NANOG, and SOX2 and by the ability to form teratomas. Teratoma formation assay was performed by injecting 1 million h-iPSCs mixed in 100 μL Matrigel into the dorsal flank of nude mice (Jackson Lab). Four weeks after the injection, tumors were surgically dissected from the mice, weighed, fixed in 4% formaldehyde, and embedded in paraffin for histology. Sections were stained with hematoxylin and eosin (H&E).

Electroporation

Electroporation was routinely used to introduce plasmids, modified mRNA and proteins into the cells as described for each experiment. Electroporation was carried out with a Neon electroporation system (ThermoFisher). Unless specified otherwise, electroporation parameters were set as 1150 v for pulse voltage, 30 ms for pulse width, 2 for pulse number, 3 mL of electrolytic buffer and 100 μL resuspension buffer R in 100 μL reaction tips (ThermoFisher, Cat No. MPK10096).

Establishment of KDR and ETV2 Knock Out h-iPSC Lines

Alt-R™ CRISPR-Cas9 system (Integrated DNA Technologies, IDT) was used to knock out KDR and ETV2 in h-iPSCs. Briefly, guide RNA (gRNA) was prepared by mixing crRNA (Table 1) and tracrRNA (IDT, Cat No. 1072533) to a final duplex concentration of 40 μM. Ribonucleoprotein (RNP) complex was prepared with 1 μL volume of 61 μM Cas9 protein (IDT, Cat No. 1074181) complexed with 2.5 μL of gRNA for 15 min at room temperature. Following incubation, RNP complexes were diluted with 100 μL R buffer and mixed with one million pelleted h-iPSCs for electroporation. Two days later, h-iPSCs were dissociated into single cells and plated at 2,000 cells per 10 cm dish in mTeSR1 supplemented with CloneR (STEMCELL, Cat No. 5888). Single cells were able to grow and form single visible colonies after 10 days. 48 colonies were randomly picked based on morphology and were then mechanically disaggregated and replated into individual wells of 48-well plates. Colonies were then expanded in culture as described above. To validate the knock out genes in each clone, genomic DNA templates were prepared by lysing cells in QuickExtract DNA extraction solution (Lucigen, Cat No. QE0905T). Target regions were amplified by using specific PCR primers (Table 1) and KAPA HiFi HotStart PCR kit (KAPA Biosystems, Cat No. KK2601). Sanger sequencing (Genewiz) was performed to identify mutant clones.

TABLE 1
Sequences of gRNA, PCR primers, and sequencing primers
used in CRISPR-Cas9 gene knockdown

| | | | | | Sanger |
|---|---|---|---|---|---|
| Target | gRNA | | PCR forward | PCR reverse | sequencing |
| Gene | sequence | PAM | primer | primer | primer |
| ETV2 | ACGGACTGT | GGG | CACTCGGGAT | GTTCGGAGCAA | GTTCGGAG |
| | ACCATTTCG | | CCGTTACTCC | ACGGTGAGA | CAAACGGT |
| | TG (SEQ ID | | (SEQ ID NO: 11) | (SEQ ID NO: 12) | GAGA (SEQ |
| | NO: 10) | | | | ID NO: 13) |
| KDR | GAGCCTACA | CGG | CAAGCCCTTT | ATTAATTTTTC | ATTAATTTT |
| | AGTGCTTCT | | GTTGTACTCA | AGGGGACAGA | TCAGGGGA |
| | AC (SEQ ID | | ATTCT (SEQ ID | GGGA (SEQ ID | CAGAGGGA |
| | NO: 14) | | NO: 15) | NO: 16) | (SEQ ID |
| | | | | | NO: 17) |

Establishment of h-iPSC Line Expressing GFP

h-iPSCs were dissociated and filtered through 40 m cell strainer to get single cells. For electroporation, 1 million h-iPSCs were resuspended in 100 μL buffer mixed with 2 g PB-EF1A-GFP-puro plasmid (VectorBuilder) and 1 μg transposase plasmid (VectorBuilder). The electroporated cells were then plated on a 35-mm Matrigel-coated dish in mTeSR1 medium with 10 μM Y27632. After 48 hours, culture medium was replaced by mTeSR1 medium with 10 μg/mL puromycin (Sigma, Cat No. P8833) and changed daily for 3 to 4 days.

Modified mRNA Synthesis and Formulation

Chemically modified mRNA encoding ETV2 (modRNA(ETV2)) was generated by TriLink BioTechnologies, LLC. In brief, modRNA(ETV2) was synthesized in vitro by T7 RNA polymerase-mediated transcription from a linearized DNA template, which incorporates the 5′ and 3′ UTRs and a poly-A tail. Specifically, the sequence used for ETV2, transcript variant 1 (NM_014209.3); ORF:

(SEQ ID NO: 1)
ATGGACCTGTGGAACTGGGATGAGGCATCCCCACAGGAAGTGCCTCCAGG
GAACAAGCTGGCAGGGCTTGAAGGAGCCAAATTAGGCTTCTGTTTCCCTG
ATCTGGCACTCCAAGGGGACACGCCGACAGCGACAGCAGAGACATGCTGG
AAAGGTACAAGCTCATCCCTGGCAAGCTTCCCACAGCTGGACTGGGGCTC
CGCGTTACTGCACCCAGAAGTTCCATGGGGGGCGGAGCCCGACTCTCAGG
CTCTTCCGTGGTCCGGGGACTGGACAGACATGGCGTGCACAGCCTGGGAC
TCTTGGAGCGGCGCCTCGCAGACCCTGGGCCCCGCCCCTCTCGGCCCGGG
CCCCATCCCCGCCGCCGGCTCCGAAGGCGCCGCGGGCCAGAACTGCGTCC
CCGTGGCGGGAGAGGCCACCTCGTGGTCGCGCGCCCAGGCCGCCGGGAGC
AACACCAGCTGGGACTGTTCTGTGGGGCCCGACGGCGATACCTACTGGGG
CAGTGGCCTGGGCGGGGAGCCGCGCACGGACTGTACCATTTCGTGGGGCG
GGCCCGCGGGCCCGGACTGTACCACCTCCTGGAACCCGGGGCTGCATGCG
GGTGGCACCACCTCTTTGAAGCGGTACCAGAGCTCAGCTCTCACCGTTTG
CTCCGAACCGAGCCCGCAGTCGGACCGTGCCAGTTTGGCTCGATGCCCCA
AAACTAACCACCGAGGTCCCATTCAGCTGTGGCAGTTCCTCCTGGAGCTG
CTCCACGACGGGGCGCGTAGCAGCTGCATCCGTTGGACTGGCAACAGCCG
CGAGTTCCAGCTGTGCGACCCCAAAGAGGTGGCTCGGCTGTGGGGCGAGC
GCAAGAGAAAGCCGGGCATGAATTACGAGAAGCTGAGCCGGGGCCTTCGC
TACTACTATCGCCGCGACATCGTGCGCAAGAGCGGGGGGCGAAAGTACAC
GTACCGCTTCGGGGGCCGCGTGCCCAGCCTAGCCTATCCGGACTGTGCGG
GAGGCGGACGGGGAGCAGAGACACAATAA; 1029 bp

was cloned into the mRNA expression vector pmRNA, which contains a T7 RNA polymerase promoter, an unstructured synthetic 5′ UTR, a multiple cloning site, and a 3′ UTR that was derived from the mouse α-globin 3′ gene. In vitro transcriptional (IVT) reaction (1 mL-scale) was performed to generate unmodified mRNA transcripts with wild type bases and a poly-A tail. Co-transcriptional capping with CleanCap Cap1 AG trimer yields a naturally occurring Cap1 structure. DNase treatment was used to remove DNA template. 5′-triphosphate were removed by phosphatase treatment to reduce innate immune response. After elution through silica membrane, the purified RNA was dissolved in RNase-free sodium citrate buffer (1 mM, pH 6.4).

Differentiation of h-iPSCs into h-iECs

The following protocols were used for differentiation.

S1-modETV2 protocol (4 days)—h-iPSCs were dissociated into single cells with TrypLE select and plated on Matrigel at a density of 60,000 cells/cm² in mTeSR1 medium with 10 μM Y27632. After 24 h, the medium was changed to S1 medium consisting of basal medium supplemented with 6 μM CHIR99021. Basal medium was prepared by adding 1× GlutaMax supplement and 60 μg/mL L-Ascorbic acid into Advanced DMEM/F12. After 48 h, h-MPCs were dissociated into single cells and then transfected with modRNA(ETV2) by either electroporation or lipofection. For electroporation, 2 million h-MPCs were resuspended in 100 μL buffer mixed with 1 μg modETV2. Electroporated cells were then seeded on a 60-mm Matrigel-coated dish in modETV2 medium consisting of basal medium supplemented with 50 ng/mL VEGF-A, 50 ng/mL FGF-2, 10 ng/mL EGF and 10 μM SB431542. For lipofection, 3 μL lipofectamine RNAiMax (ThermoFisher, Cat No. 13778030) were diluted in 50 μL Opti-MEM (ThermoFisher, Cat No. 31985062) and 0.6 μg modRNA(ETV2) diluted in another 50 μL Opti-MEM. Lipofectamine and modRNA(ETV2) were then mixed and incubated for 15 min at room temperature. The lipid/RNA complex was added to 0.5 million h-MPCs in modETV2 medium and transfected cells were then seeded on a 35-mm Matrigel-coated dish. Upon transfection (electroporation or lipofection), cells were cultured for another 48 h before purification. Medium was changed every day throughout this protocol (Table 2). modRNA encoding GFP (TriLink, Cat No. L-7601) at a concentration of 0.2 μg per million h-MPCs served as negative control.

TABLE 2
S1-modETV2 protocol

| | | | | Basal |
|---|---|---|---|---|
| Stages | Days | Supplements | | medium |
| S0 | 0 | Y27632 | (Seed 60,000 h-iPSCs/cm² on | mTeSR1 |
| | | 10 μM | matrigel-coated 6-well plates) | |

| | | | |
|---|---|---|---|
| S1 | 1 | CHIR99021 | Advanced |
| | | 6 μM | DMEM/ |
| | 2 | Repeat Day 1 | F12 |
| | | | Ascorbic |
| | | | acid |
| | | | 60 μg/mL |
| | | | GlutaMax |
| | | | 1× |

h-MPCs were dissociated into single cells and then transfected with
modRNA(ETV2) by either electroporation or lipofection

| | | | | | |
|---|---|---|---|---|---|
| ModETV2 | 3 | VEGF- | FGF-2 | EGF | SB431542 |
| | | A 50 | 50 | 10 | 10 μM |
| | | ng/mL | ng/mL | ng/mL | |

| | |
|---|---|
| 4 | Repeat Day 3 |

Early modETV2 protocol (2 days)—h-iPSCs were dissociated into single cells and then transfected with modRNA(ETV2) by electroporation. For electroporation, 2 million h-iPSCs were resuspended in 100 μL buffer and mixed with 1.5 μg modRNA(ETV2). Electroporated cells were then plated on a 60-mm Matrigel-coated dish in mTeSR1 medium with 10 μM Y27632. After 24 h, the medium was changed to mTeSR1 medium with 10 μM SB431542 for another 24 h.

S1-S2, method #1 (4 days)—h-iPSCs were dissociated into single cells with TrypLE select and plated on Matrigel at a density of 60,000 cells/cm² in mTeSR1 medium with 10 μM Y27632. After 24 h, the medium was changed to S1 medium consisting of basal medium supplemented with 6 μM CHIR99021. Basal medium was prepared by adding 1× GlutaMax supplement and 60 μg/mL L-Ascorbic acid into Advanced DMEM/F12. After 48 h, the differentiation medium was changed to S2 medium for 48 h. S2 medium consisted of basal medium supplemented with 50 ng/mL VEGF-A, 50 ng/mL FGF-2, 10 ng/mL EGF and 10 μM SB431542. Medium was changed every day throughout this protocol. Details for this protocol are provided in Table 3.

TABLE 3
S1-S2 method #1

| | | | | | | Basal |
|---|---|---|---|---|---|---|
| Stages | Days | Supplements | | | | medium |
| S0 | 0 | Y27632 | (Seed 60,000 h-iPSCs/cm² on | | | mTeSR1 |
| | | 10 μM | matrigel-coated 6-well plates) | | | |
| S1 | 1 | CHIR99021 | | | | Advanced |
| | | 6 μM | | | | DMEM/ |
| | 2 | Repeat Day 1 | | | | F12 |
| | | | | | | Ascorbic |
| | | | | | | acid |
| | | | | | | 60 μg/mL |
| S2 | 3 | VEGF-A | FGF-2 | EGF | SB431542 | GlutaMax |
| | | 50 ng/mL | 50 | 10 ng/mL | 10 μM | 1× |
| | | | ng/mL | | | |

| | |
|---|---|
| 4 | Repeat Day 3 |

S1-S2, method #2 (4 days)—h-iPSCs were dissociated into single cells with TrypLE select and plated on Matrigel at a density of 60,000 cells/cm² in mTeSR1 medium with 10 μM Y27632. After 24 h, the medium was changed to STEMdiff APEL2 medium supplemented with 6 μM CHIR99021. After 48 h, the differentiation medium was changed to S2 medium for 48 h. S2 medium consisted of STEMdiff APEL2 medium supplemented with 50 ng/mL VEGF-A, 10 ng/mL FGF-2, and 25 ng/mL BMP4. Medium was changed every day throughout this protocol. Details for this protocol are provided in Table 4. This protocol was adopted from Harding et al. Stem Cells 35, 909-919 (2017).

TABLE 4
S1-S2 method #2

| Stages | Days | Supplements | | | Basal medium |
|---|---|---|---|---|---|
| S0 | 0 | Y27632 | (Seed 60,000 h-iPSCs/cm² | | mTeSR1 |
| | | 10 μM | on matrigel-coated 6-well | | |
| | | | plates) | | |
| S1 | 1 | CHIR99021 | | | STEMdiff |
| | | 6 μM | | | APEL2 Media |
| | 2 | Repeat Day 1 | | | |
| S2 | 3 | VEGF-A | FGF-2 | BMP4 | |
| | | 50 ng/mL | 10 ng/mL | 25 ng/mL | |

| | |
|---|---|
| 4 | Repeat Day 3 |

S1-S2, method #3 (4 days)—h-iPSCs were dissociated into single cells with TrypLE select and plated on Matrigel at a density of 60,000 cells/cm² in mTeSR1 medium with 10 μM Y27632. After 24 h, the medium was changed to basal medium supplemented with 1 μM CP21R7 and 20 ng/mL BMP4. Basal medium was prepared by adding 1×B27 supplement and 1×N2 into DMEM/F12. After 48 h, the differentiation medium was changed to S2 medium for 48 h. S2 medium consisted of StemPro-34 SFM supplemented with 50 ng/mL VEGF-A, and 10 μM DAPT. Medium was changed every day throughout this protocol. Details for this protocol are provided in Table 5. This protocol was adopted from Sahara et al. Cell Res 24, 820-841 (2014).

TABLE 5
S1-S2, method #3

| | | | | Basal |
|---|---|---|---|---|
| Stages | Days | Supplements | | medium |
| S0 | 0 | Y27632 | (Seed 60,000 h-iPSCs/cm² | mTeSR1 |
| | | 10 μM | on matrigel-coated 6-well | |
| | | | plates) | |
| S1 | 1 | CP21R7 | BMP4 | DMEM/F12 |
| | | 1 μM | 20 ng/mL | 1 × B27 |
| | 2 | Repeat Day 1 | | 1 × N2 |
| S2 | 3 | VEGF-A | DAPT | StemPro-34 |
| | | 50 ng/mL | 10 μM | |

| | |
|---|---|
| 4 | Repeat Day 3 |

S1-S2, method #4 (4 days)—h-iPSCs were dissociated into single cells with TrypLE select and plated on Matrigel at a density of 60,000 cells/cm² in mTeSR1 medium with 10 μM Y27632. After 24 h, the medium was changed to basal medium supplemented with 8 μM CHIR99021. Basal medium was prepared by adding 1×B27 supplement and 1×N2 into DMEM/F12. After 48 h, the differentiation medium was changed to S2 medium for 48 h. S2 medium consisted of StemPro-34 SFM supplemented with 200 ng/mL VEGF-A, and 2 μM forskolin. Medium was changed every day throughout this protocol. Details for this protocol are provided in Table 6. This protocol was adopted from Patsch et al. Nat Cell Biol 17, 994-1003 (2015).

TABLE 6
S1-S2, method #4

| Stages | Days | Supplements | | Basal medium |
|---|---|---|---|---|
| S0 | 0 | Y27632 | (Seed 60,000 h-iPSCs/cm² | mTeSR1 |
| | | 10 μM | on matrigel-coated 6-well | |
| | | | plates) | |
| S1 | 1 | CHIR99021 | | DMEM/F12 |
| | | 8 μM | | 1 × B27 |
| | 2 | Repeat Day 1 | | 1 × N2 |
| S2 | 3 | VEGF-A | Forskolin | StemPro-34 |
| | | 200 ng/mL | 2 μM | |

| | |
|---|---|
| 4 | Repeat Day 3 |

Purification and Expansion of h-iECs

At indicated time points after differentiation, h-iECs were dissociated into single cells and sorted into CD31+ and CD31− cells using magnetic beads coated with anti-human CD31 antibodies (DynaBeads, ThermoFisher, Cat No. 11155D). The purified CD31+ h-iECs were then expanded in culture on 10-cm dishes coated with 1% gelatin. Culture medium for h-iECs was prepared by adding Endothelial Cell Growth medium 2 kit supplements into basal medium (except for hydrocortisone, PromoCell, Cat No. C22111) with 1×GlutaMax supplement and 10 μM SB431542.

RNA-Seq Analysis

The following groups were analysed: h-iPSCs, human ECFCs, and h-iECs generated with three protocols: S1-S2, S1-modETV2, and early modETV2. Each group consists of 3 biological replicates. Total RNA from h-iECs which have been expanded for 7 days was extracted using Rneasy Mini Kit (Qiagen) following the manufacturer's protocol. RNA quantity and quality were checked with nanodrop and Agilent Bioanalyzer instrument. Libraries were prepared and sequenced by GENEWIZ (NJ, USA). Library preparation involved mRNA enrichment and fragmentation, chemical fragmentation, first and second strand Cdna synthesis, end repair and 5′ phosphorylation, Da-tailing, adaptor ligation and PCR enrichment. The libraries were then sequenced using Illumina HiSeq2500 platform (Illumina, CA) using 2×150 paired end configuration. The raw sequencing data (FASTQ files) was examined for library generation and sequencing quality using FastQC (bioinformatics.babraham.ac.uk/projects/fastqc/) to ensure data quality was suitable for further analysis. Reads were aligned to UCSC hg38 genome using the STAR aligner (Dobin, A. et al. Bioinformatics 29, 15-21 (2013)). Alignments were checked for evenness of coverage, rRNA content, genomic context of alignments, complexity, and other quality checks using a combination of FastQC, Qualimap (Garcia-Alcalde, F. et al. Bioinformatics 28, 2678-2679 (2012).) and MultiQC (Ewels, P., et al. Bioinformatics 32, 3047-3048 (2016)). The expression of the transcripts was quantified against the Ensembl release GRCh38 transcriptome annotation using Salmon. These transcript abundances were then imported into R (version 3.5.1) and aggregated to the gene level with tximport. Differential expression at the gene level was called with DESeq2 (Love, et al. Genome Biol. 15, 550 (2014)). Pairwise differential expression analysis between groups was performed using Wald significance test. The P values was corrected for multiple hypothesis testing with the Benjamini-Hochberg false-discovery rate procedure (adjusted P value). Genes with adjusted P value <0.05 were considered significantly different. Hierarchical clustering and PCA analysis were performed on DESeq2 normalized, rlog variance stabilized reads. All samples comparison was performed using Likelihood Ratio Test (LRT). Heat maps of the differential expressed genes and enriched gene sets were generated with pheatmap package. Functional enrichment of differential expressed genes, using gene sets from Gene Ontology (GO), was determined with Fisher's exact test as implemented in the clusterProfiler package. The RNA-Seq datasets are deposited online with SRA accession number: PRJNA509218.

Chemicals and Media Components

Chemicals and media components used herein are shown in Table 7.

TABLE 7
Chemicals and media components

| Components | Vendor | Cat. No. |
|---|---|---|
| mTesR1 | STEMCELL | 85870 |
| Y27632 | Selleckchem | S1049-50 mg |
| Matrigel | Corning | 354277 |
| Advanced DMEM/F12 | ThermoFisher | 12634010 |
| GlutaMax | ThermoFisher | 35050061 |
| Ascorbic acid phosphate | Millipore-Sigma | A8960 |
| CHIR99021 | Millipore-Sigma | SML1046 |
| VEGF-A | PeproTech | 100-20 |
| EGF | PeproTech | AF-100-15 |
| FGF-2 | PeproTech | 100-18B |
| SB431542 | Selleckchem | S1067-50 mg |
| STEMdiff APEL2 Media | STEMCELL | 05275 |
| DMEM/F12 | ThermoFisher | 11330032 |
| N2 | ThermoFisher | 17502048 |
| B27 | ThermoFisher | 17504044 |
| CP21R7 | Selleckchem | S7954-5 mg |
| BMP4 | PeproTech | 120-05ET |
| StemPro-34 | ThermoFisher | 10639011 |
| DAPT | Selleckchem | S2215-25 mg |
| Forskolin | Millipore-Sigma | F3917-10 MG |
| EGM2 kit | PromoCell | C-22111 |
| Gelatin | Millipore-Sigma | G2500-500G |

Flow Cytometry

Cells were dissociated into single-cell suspensions using TrypLE and washed with PBS supplemented with 100BSA and 0.2 mM EDTA. In indicated experiments, cells were stained with flow cytometry antibodies and analyzed using a Guava easyCyte 6HT/2L flow cytometer (Millipore Corporation, Billerica, Mass.) and FlowJo software (Tree Star Inc., Ashland, Oreg.). Antibody labeling was carried out for 10 min on ice followed by 3 washes with PBS buffer. Antibody information is detailed in Table 8.

TABLE 8
List of antibodies used in the study

| Antibody | Vendor | Cat.No. | Clone | Dilution |
|---|---|---|---|---|
| R-PE anti-CD31 | Ancell | 180-050 | 158-2B3 | 1:100 (FC) |
| APC anti-CD31 | Biolegend | 303116 | WM-59 | 1:100 (FC) |
| APC anti-human CD309 | Biolegend | 359916 | 7D4-6 | 1:100 (FC) |
| (VEGFR2) | | | | |
| PE anti-human CD309 | Biolegend | 359903 | 7D4-6 | 1:100 (FC) |
| (VEGFR2) | | | | |
| PE anti-CD144(VE-CAD) | ThermoFisher | 12-1449-80 | 16B1 | 1:100 (FC) |
| PE anti-TRA-1-81 | ThermoFisher | 12-8883-80 | | 1:100 (FC) |
| PE anti-human CD62E (E- | ThermoFisher | 12-0627-41 | P2H3 | 1:100 (FC) |
| Selectin) | | | | |
| PE anti-CD54 (ICAM-1) | ThermoFisher | 12-0549-41 | HA58 | 1:100 (FC) |
| PE anti-CD106 (VCAM-1) | Biolegend | 305805 | | 1:100 (FC) |
| Rabbit anti-smooth muscle | Abcam | Ab133567 | EPR5336(B) | 1:200 (IF) |
| myosin heavy chain 11 | | | | |
| Rabbit anti-alpha smooth | Abcam | Ab5694 | | 1:300(IHC-P) |
| muscle actin | | | | |
| Mouse anti-alpha smooth | Sigma | A2547 | 1A4 | 1:300(IHC-P) |
| muscle actin | | | | |
| Rabbit anti-SM22-alpha | Abcam | Ab14106 | | 1:200(IF) |
| Mouse anti-VE-cadherin | Santa Cruz | Sc-9989 | F-8 | 1:200(IF) |
| Rabbit anti-human Von | DAKO | A0082 | | 1:200(IF) |
| Willebrand Factor | | | | |
| Mouse anti-human CD31 | Agilent | M082329-2 | JC70A | 1:50(IHC-P) |
| | | | (human specific) | 1:200(IF) |
| Mouse anti-human CD31 | Abcam | Ab9498 | JC70A | 1:200(IHC-P) |
| | | | (human specific) | |
| Rabbit anti-human CD31 | Abcam | Ab76533 | EPR3094 | 1:200(IHC-P) |
| | | | (human specific) | |
| Rhodamine labeled Ulex | Vector | RL-1062 | (Human specific) | 1:100(IHC-P) |
| Europaeus Agglutinin I | | | | 1:200(IF) |
| (UEA I) | | | | |
| Mouse anti-human | Abcam | Ab8069 | V9 | 1:300 (IHC-P) |
| vimentin | | | (human specific) | |
| Rabbit anti-GFP | Abcam | Ab183734 | EPR14104 | 1:200 (IHC-P) |
| Rabbit anti-ETV2 | Abcam | Ab181847 | EPR5229(3) | 1:300(IF) |
| Rabbit anti-Oct4 | Stemgent | 09-0023 | | 1:300(IF) |
| Rabbit anti-Sox2 | Stemgent | 09-0024 | | 1:300(IF) |
| Mouse anti-Klf4 | Stemgent | 09-0021 | | 1:300(IF) |
| Rabbit anti-Nanog | Stemgent | 09-0020 | | 1:300(IF) |
| Goat anti-Brachyury (T) | R&D | AF2085 | | 5 μg/mL (IF) |
| Donkey anti-mouse IgG, | ThermoFisher | A-21202 | | 1:500 (IF, IHC- |
| AlexaFluor 488 | | | | P) |
| Donkey anti-goat IgG, | ThermoFisher | A-11058 | | 1:500 (IF, IHC- |
| AlexaFluor 594 | | | | P) |
| Donkey anti-rabbit IgG, | ThermoFisher | A-21206 | | 1:500 (IF, IHC- |
| AlexaFluor 488 | | | | P) |
| Horse anti-mouse IgG | Vector | TI-2000 | | 1:400 (IF, IHC- |
| Texas Red | | | | P) |
| Horse anti-rabbit IgG | Vector | DI-1094 | | 1:400 (IF, IHC- |
| DyLight 594 | | | | P) |

Microscopy

Images were taken using an Axio Observer Z 1 inverted microscope (Carl Zeiss) and AxioVision Rel. 4.8 software. Fluorescent images were taken with an ApoTome.2 Optical sectioning system (Carl Zeiss) and 20× objective lens. Non-fluorescent images were taken with an AxioCam MRc5 camera using a 5× or 10× objective lens.

Immunofluorescence Staining

Cells were seeded in 8-well LAB-TEK chamber slides at a density of 60,000 cell s/cm². After confluency, cells were fixed in 4% paraformaldehyde (PFA), permeabilized with 0.1% Triton X-100 in PBS, and then blocked for 30 min in 5% horse serum (Vector, Cat No. 5-2000). Subsequently, cells were incubated with primary antibodies for 30 min at room temperature (RT). Cells were washed 3 times with PBS and then incubated with secondary antibodies for 30 min at RT. Cells were washed 3 times with PBS and stained with 0.5 μg/mL DAPI for 5 min. Slides were mounted with DAKO fluorescence mounting medium (Agilent, Cat No. 5302380-2). Antibody information is detailed in Table 8, above.

Spheroid Sprouting Assay

EC spheroids were generated by carefully depositing 500 h-MSCs and 500 h-iECs-GFP in 20 μL spheroid-forming medium on the inner side of a 10-cm dish lid. The spheroid-forming medium contained 0.24% (w/v) methyl cellulose (Sigma, Cat No. M0512). The lid was then turned upside down and placed on top of the plate filled with 10 mL sterile water. EC spheroids were collected after 2 days in culture and embedded in fibrin gel prepared with 5 mg/mL fibrinogen (Sigma, Cat No. F8630) and 0.5 U/mL thrombin (Sigma, Cat No. T-9549). A 100 μL-fibrin gel/spheroid solution was spotted into the center of a 35-mm glass bottom dish (MatTek, Cat No. P35G-1.5-10-C) and incubated for 10 mins at 37° C. for solidification. Gel/spheroid constructs were kept in culture for 3 days. GFP+ sprouts were imaged using an inverted fluorescence microscope and sprout lengths were measured by ImageJ.

Shear Stress Response Assay

Confluent monolayers of h-iECs in a 100-mm culture dish were subjected to orbital shear stress for 24 h at a rotating frequency of 150 rpm using an orbital shaker (VWR, Model 1000) positioned inside a cell culture incubator. After 24 h, cells were fixed in 4% PFA and stained using an anti-human VE-Cadherin antibody. Alignment of ECs was visualized using an inverted fluorescence microscope under a 10× objective. Only the cells in the periphery of the culture dish were imaged. Cell orientation angles were measured by ImageJ.

Nitric Oxide (NO) Production Assay

Cells were cultured on gelatin-coated 12-well plates (2×10⁵ cells per well) in h-iECs media. To measure nitric oxide (NO), media were changed to fresh media containing 1 μM DAF-FM (Cayman, Cat No. 18767). Cells were cultured for 30 min and then harvested for flow cytometric analysis and fluorescent imaging. In order to suppress NO production, h-iECs were cultured in the presence of 5 mM L-NAME (Cayman, Cat No. 80210) for 24 h. DAF-FM is nonfluorescent until it reacts with NO to form a fluorescent benzotriazole (FITC channel). The mean fluorescence intensities (MFIs) were measured by calculating the geometric mean in FlowJo.

Leukocyte Adhesion Molecules and Leukocyte Adhesion Assays

Cells were cultured on a gelatin-coated 48-well plate (10⁵ cells per well) in h-iEC medium. At confluency, cells were treated with or without 10 ng/mL TNF-α (Peprotech, Cat No. 300-01A) for 5 h. Cells were then lifted and treated with anti-ICAM1, anti-E-selectin or anti-VCAM1 antibodies for flow cytometry. For leukocyte adhesion assay, human HL-60 leukocytes were used. HL-60 cells were culture in leukocyte medium consisting of RPMI-1640 (ThermoFisher, Cat No. 11875093) supplemented with 20% FBS. 2×10⁵ HL-60 cells were suspended in 0.2 mL fresh leukocyte medium and added to each well. After gentle shaking for 45 min in cold room, plates were gently washed twice with cold leukocyte media. Cells were fixed in 2.5% (v/v) glutaraldehyde at RT for 30 min and then imaged. Bound leukocytes were quantified by ImageJ analysis software.

Smooth Muscle Cell Differentiation Assay

2×10⁴ h-MSCs and 5×10⁴ h-iECs were plated in one well of 8-well LAB-TEK chamber slide coated by 1% gelatin and cultured in h-iECs medium without SB431542 for 7 days. Smooth muscle cell positive cells were stained with an anti-smooth muscle myosin heavy chain 11 antibody and ECs and nucleus were stained by anti-VECAD antibody and DAPI, respectively. h-MSCs that were transduced with lentivirus to express GFP (h-MSCs-GFP) were used in indicated experiments. Antibody information is detailed in Table 8, above.

Tube Formation Assay

8×10³ h-iECs were plated in one well of 96-well plate on top of solidified Matrigel (50 μL) with h-iECs media. After 6 h, cells were incubated with 1 μM Calcein-AM (Biolegend, Cat No. 425201) for 10 min and then imaged using a fluorescence microscope. Numbers of branches were counted by ImageJ.

In Vivo Vascular Network-Forming Assay

Six-week-old NOD/SCID mice were purchased from Jackson Lab (Boston, Mass.). Mice were housed in compliance with Boston Children's Hospital guidelines, and all animal-related protocols were approved by the Institutional Animal Care and Use Committee. H-iECs were pretreated with 20 μM caspase inhibitor/Z-VAD-FMK (APExBio, Cat No. A1902) and 0.5 μM BCL-XL-BH4 (Millipore, Cat No. 197217) in h-iECs medium overnight before implantation. Briefly, h-iECs and h-MSCs (2×10⁶ total per mice, 1:1 ratio) or h-iECs alone (1×10⁶ cells per mice) were resuspended in 200 μL of pH neutral pre-gel solution containing 3 mg/mL of bovine collagen I (Trevigen, Cat No. 3442-050-01), 3 mg/mL of fibrinogen, 50 μL Matrigel (Corning, Cat No. 354234), 1 g/mL of FGF2 (Peprotech, Cat No. 100-18B) and 1 g/mL EPO (ProSpec, Cat No. CYT-201). During anesthesia, mice were firstly injected with 50 μL of 10 U/mL thrombin (Sigma, Cat No. T4648) subcutaneously and then injected with 200 μL cell-laden pre-gel solution into the same site. All experiments were carried out in 5 mice and explants were harvested after 1 week and 1 month.

Histology and Immunofluorescence Staining

Explanted grafts were fixed overnight in 10% buffered formalin, embedded in paraffin and sectioned (7-μm-thick). Microvessel density was reported as the average number of erythrocyte-filled vessels (vessels/mm²) in H&E-stained sections from the middle of the implants as previously described. For immunostaining, sections were deparaffinized and antigen retrieval was carried out with citric buffer (10 mM sodium citrate, 0.05% Tween 20, pH 6.0). Sections were then blocked for 30 min in 5% horse serum and incubated with primary and secondary antibodies for 30 min at RT. Fluorescent staining was performed using fluorescently-conjugated secondary antibodies followed by DAPI counterstaining. Human-specific anti-CD31 antibody and UEA-1 lectin were used to stain human blood vessels. Perivascular cells were stained by anti-alpha smooth muscle actin antibody. Primary and secondary antibodies are detailed in Table 8, above. The Click-It Plus TUNEL assay (ThermoFisher, Cat No. C10617) was used to detect apoptotic cells in tissue.

Quantitative RT-PCR

Quantitative RT-PCR (qRT-PCR) was carried out in RNA lysates prepared from cells in culture. Total RNA was isolated with a RNeasy kit (Qiagen, Cat No. 74106) and cDNA was prepared using reverse transcriptase III (ThermoFisher, Cat No. 4368814), according to the manufacturer's instructions. Quantitative PCR was performed using SRBR Green Master Mix (ThermoFisher, Cat No. A25776), and detection was achieved using the StepOnePlus Real-time PCR system thermocycler (Applied Biosystems). Expression of target genes was normalized to GAPDH. Real-time PCR primer sequences are listed in Table 9.

TABLE 9
Sequences of primers used for qRT-PCR

| Gene | Forward (5′→3′) | # | Reverse (5′→3′) | # |
|---|---|---|---|---|
| POU5F1 | GGGCTCTCCCATGCATTCAAAC | 18 | CACCTTCCCTCCAACCAGTTGC | 19 |
| MIXL1 | ACGTCTTTCAGCGCCGAACAG | 20 | TTGGTTCGGGCAGGCAGTTCA | 21 |
| TBXT | GTGCTGTCCCAGGTGGCTTACAGATG | 22 | CCTTAACAGCTCAACTCTAACTACTTG | 23 |
| ACTA2 | TGACAATGGCTCTGGGCTCTGTAA | 24 | TTCGTCACCCACGTAGCTGTCTTT | 25 |
| GAPDH | CATGTTCGTCATGGGTGTGAACCA | 26 | ATGGCATGGACTGTGGTCATGAGT | 27 |
| ERG | AACCATCTCCTTCCACAGTGCCCAAA | 28 | TTTGCAAGGCGGCTACTTGTTGGT | 29 |
| ETV2 | CCGACGGCGATACCTACTG | 30 | CGGTGGTTAGTTTTGGGGCAT | 31 |
| NOS3 | TGACCCTCACCGCTACAACATCCT | 32 | CGTTGATTTCCACTGCTGCCTTGTCT | 33 |
| CLDN5 | CTCTGCTGGTTCGCCAACAT | 34 | CAGCTCGTACTTCTGCGACA | 35 |
| ENG | CGGTGGTCAATATCCTGTCGAG | 36 | AGGAAGTGTGGGCTGAGGTAGA | 37 |
| TEK | GCTTGCTCCTTTCTGGAACTGT | 38 | CGCCACCCAGAGGCAAT | 39 |
| PECAM1 | CACCTGGCCCAGGAGTTTC | 40 | AGTACACAGCCTTGTTGCCATGT | 41 |
| CDH5 | GAACCCAAGATGTGGCCTTTAG | 42 | GATGTGACAACAGCGAGGTGTAA | 43 |
| VWF | GTCGAGCTGCACAGTGACATG | 44 | GCACCATAAACGTTGACTTCCA | 45 |
| KDR | ATCCAGTGGGCTGATGACCAAGAA | 46 | ACCAGAGATTCCATGCCACTTCCA | 47 |

#, SEQ ID NO:

Statistical Analyses

Unless otherwise stated, data were expressed as mean±standard deviation of the mean (s.d.). For comparisons between two groups, means were compared using unpaired two-tailed Student's t-tests. Comparisons between multiple groups were performed by ANOVA followed by Bonferroni's post-test analysis. Samples size, including number of mice per group, was chosen to ensure adequate power and were based on historical data. No exclusion criteria were applied for all analyses. No specific methods of randomization were applied to group/animal allocation. Investigators were not blinded to group allocation. All statistical analyses were performed using GraphPad Prism v.5 software (GraphPad Software Inc.). P<0.05 was considered statistically significant.

### Example 1. Rapid and Highly Efficient Differentiation of Human h-iPSCs into h-iECs

We developed an exemplary two-dimensional, feeder-free, and chemically defined protocol that relied on a timely transition of h-iPSCs through two distinct stages, each lasting about 48 h. First is the conversion of h-iPSCs into h-MPCs. This step is similar to that in the standard S1-52 differentiation protocol and thus is mediated by the activation of Wnt and Nodal signaling pathways using the glycogen synthase kinase 3 (GSK-3) inhibitor CHIR99021 (FIG. 1a). Second, we converted the h-MPCs into h-iECs. This step is substantially different than the S1-52 protocol which relies on activation of endogenous ETV2 via VEGF signaling. In contrast, our protocol used chemically modified RNA (modRNA) to deliver exogenous ETV2 to h-MPCs via either electroporation or lipofection (FIG. 1a).

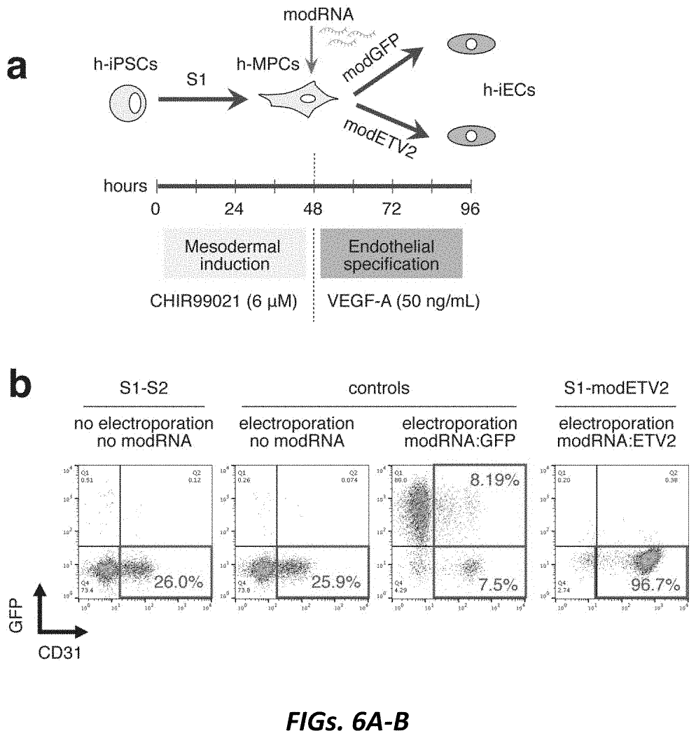

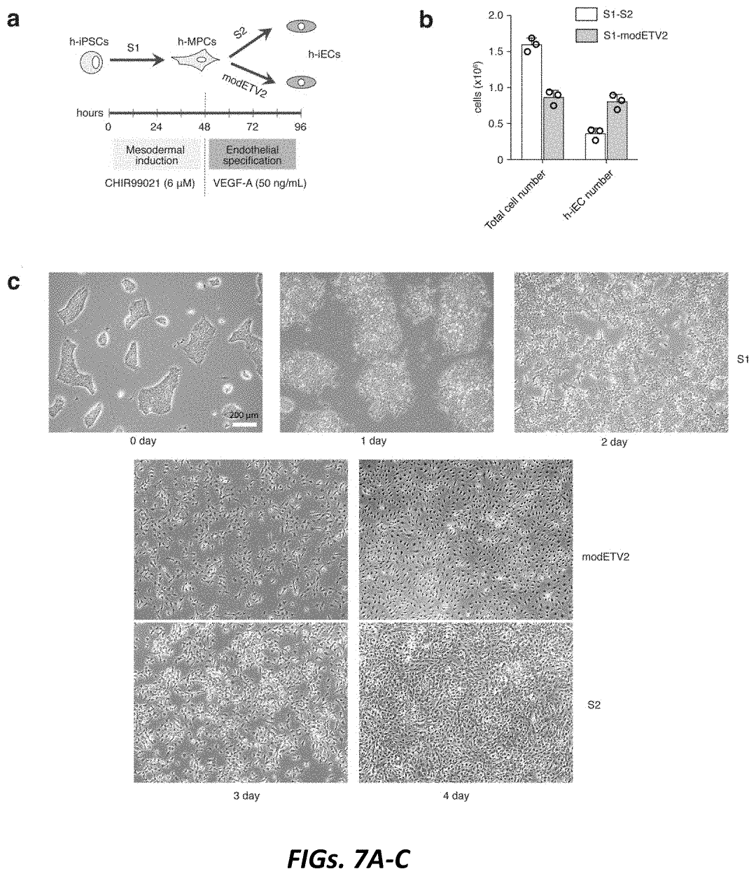

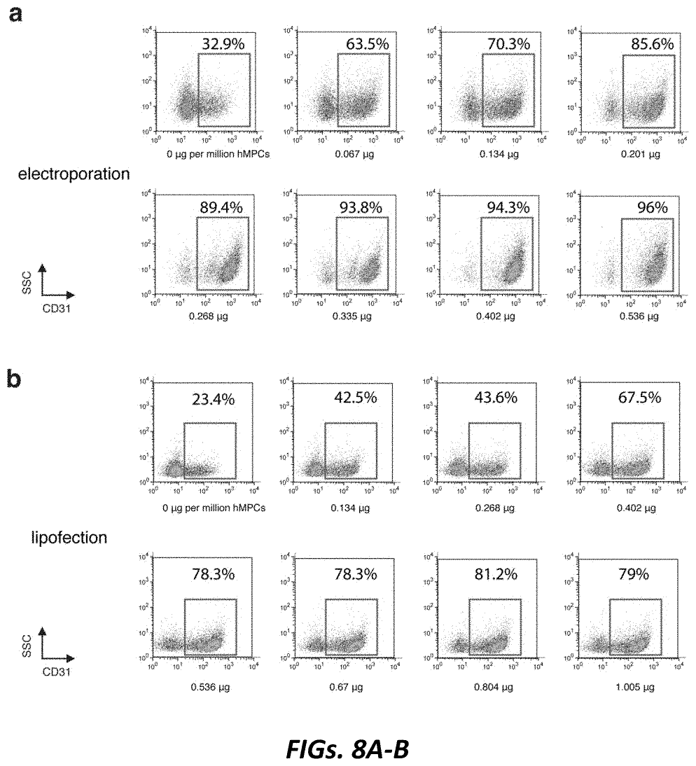

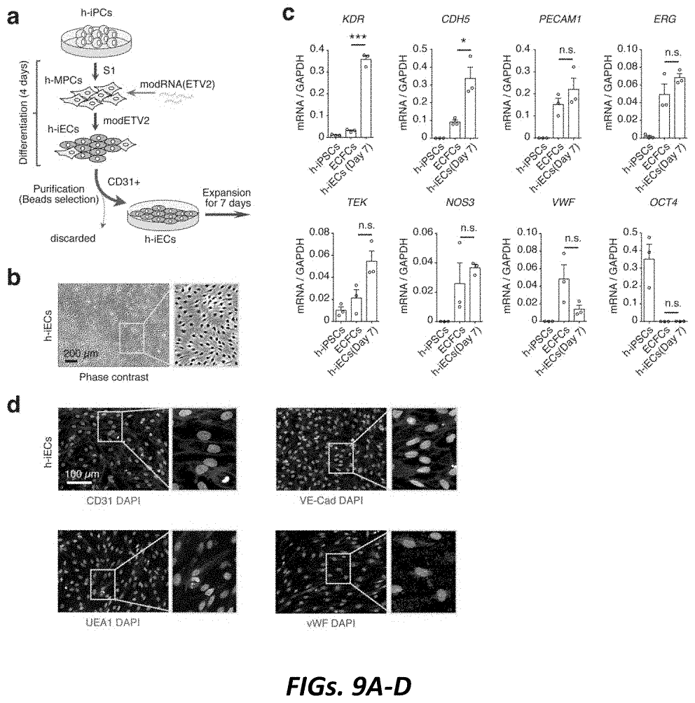

This customized two-step protocol (herein referred to as S1-modETV2) rapidly and uniformly converted human h-MPCs into h-iECs. Indeed, 48 h after transfection of h-MPCs with modRNA(ETV2), 95% of the cells were endothelial (VE-Cadherin+/CD31+ cells; FIG. 1b) (see FIG. 6A-B for controls accounting for electroporation with no modRNA and with modRNA(GFP)). In contrast, conversion during the standard S2 step (no modRNA) was slower and significantly less efficient, with less than 30% VE-Cadherin+/CD31+ cells at the same time point (FIG. 1b) (note that total cell number at day 4 was actually higher in the S1-52 method; however, h-iEC number was higher in the S1-modETV2 method due to the high efficiency; FIG. 7A-C). Conversion efficiency was dependent on the amount of modRNA(ETV2) used. Titration analysis revealed that above 0.5 μg of modRNA(ETV2) per 10⁶ h-iPSCs, the percentage of h-iECs at 96 h was consistently 95% (using electroporation) and 75% (lipofection) (FIG. 1c; FIG. 8A-B). The resulting h-iECs displayed typical cobblestone-like morphology, did not express the pluripotent marker OCT4, expressed numerous EC markers at the mRNA and protein levels, and showed affinity for the binding of Ulex europaeus agglutinin I (UEA-1) lectin (FIG. 9A-D).

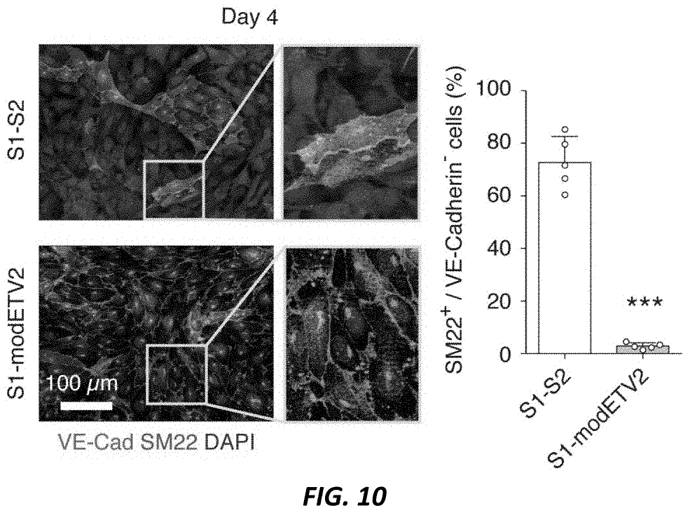

Transfection with modRNA(ETV2) enabled rapid, transient, and uniform expression of ETV2, in contrast to delayed and sparse expression with the S1-52 method (FIG. 1d). Broad expression of ETV2, in turn, resulted in uniform CD31 expression by 96 h (FIG. 1d). During the S1-S2 protocol, the presence of non-endothelial VE-Cadherin-/SM22+cells was prominent at 96 h (FIG. 10). However, the occurrence of VE-Cadherin-/SM22+ cells was significantly reduced in our S1-modETV2 protocol (<3%), suggesting a more effective avoidance of alternative non-endothelial differentiation pathways (FIG. 10).

### Example 2. Differentiation Reproducibility with Clonal h-iPSC Lines from Various Cellular Origins

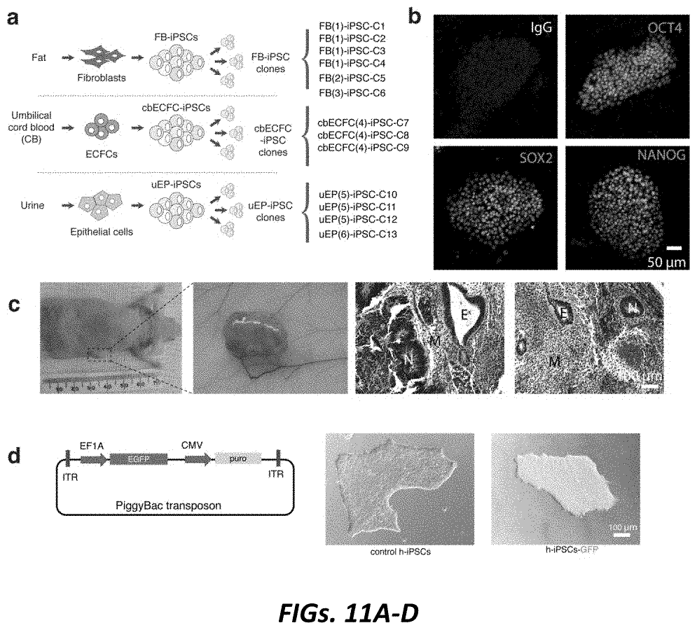

Current S1-S2 differentiation protocols lack consistency between different h-iPSC lines. To address this limitation, we generated multiple human clonal h-iPSC lines from three distinct cellular origins corresponding to subcutaneous dermal fibroblasts (FB), umbilical cord blood-derived endothelial colony-forming cells (cbECFC), and urine-derived epithelial cells (uEP) (FIG. 11a). All h-iPSCs were generated with a non-integrating episomal approach and validated by expression of pluripotent transcription factors OCT4, NANOG, and SOX2; and capacity to form teratomas in immunodeficient mice (FIG. 1b-c).

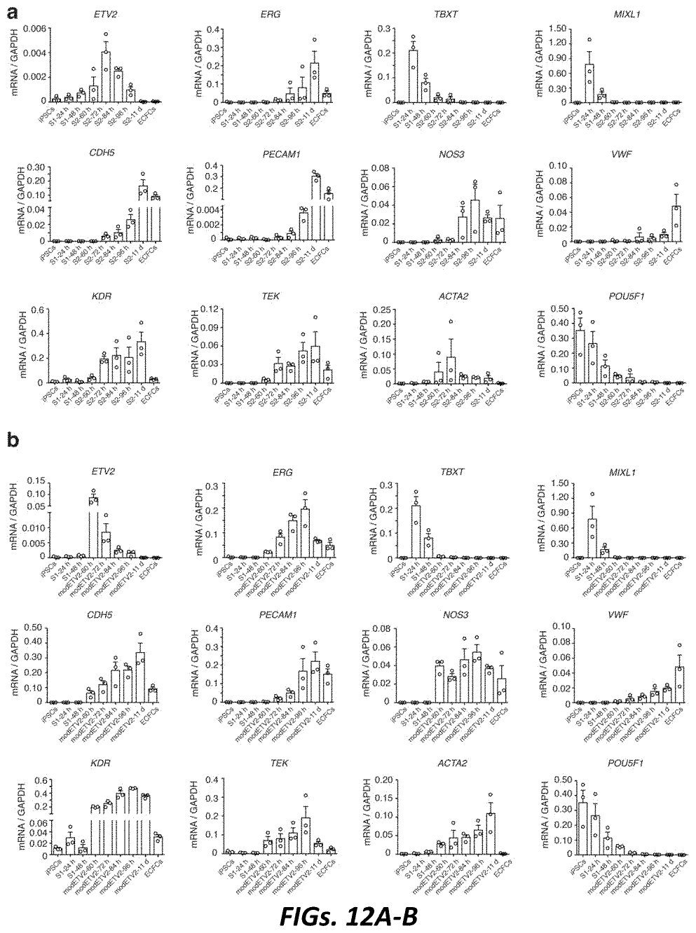

We generated 13 clones (referred to as C1-C13) to collectively represent variations due to different individual donors, cellular origins, and clone selection (FIG. 12A-B). All clones were subjected to both S1-S2 and S1-modETV2 differentiation protocols. As expected, the S1-S2 protocol produced a wide variation in efficiency, with h-iECs ranging from <1% to 24% (FIG. 1e-f). In addition, there were noticeable inconsistencies between clones from similar cellular origins but different donors (e.g., C2 vs. C5; and C10 vs. C13) and even between genetically-identical clones derived from the same h-iPSC line (e.g., C2 vs. C4; C8 vs. C9; and C10 vs. C12) (FIG. 1e). In contrast, differentiation under the S1-modETV2 protocol produced significantly higher efficiencies and eliminated inconsistencies between clones. Indeed, in all 13 clones, the percentage of CD31+ h-iECs at 96 h ranged between 88-96% irrespective of the donor and cellular origin from which the h-iPSC clones were derived, and there were no statistical differences in efficiency (FIG. 1e-f).

To further corroborate these findings, we examined three additional S1-S2 methodologies corresponding to protocols described by Harding et al. 2017⁷ (Method #2), Sahara et al. 2014 s (Method #3), and Patsch et al. 2015⁴ (Method #4). These methods were compared to our S1-S2 method (referred to as Method #1 in FIG. 1g) and the S-modETV method (see details for all methods in Tables 2-6). For this comparison, we used three independent h-iPSC clones corresponding to three different cellular origins: dermal fibroblasts (clone FB(1)-iPSC-C3); umbilical cord blood-derived endothelial colony-forming cells (cbECFC(4)-iPSC-C9), and urine-derived epithelial cells (uEP(5)-iPSC-C11) (FIG. 1g). Examination of the efficiency at 96 h revealed that all four S1-S2 differentiation protocols produced significantly lower efficiencies than the S1-modETV method. Moreover, depending on the h-iPSC clone used, there was a wide variation in efficiency among the four S1-2 methods, with h-iECs ranging from <2% to 45% (FIG. 1g). In contrast, differentiation under the S1-modETV2 protocol produced significantly higher efficiencies (89-95%) and eliminated inconsistencies between clones.

### Example 3. Inefficient Activation of Endogenous ETV2 in Intermediate h-MPCs

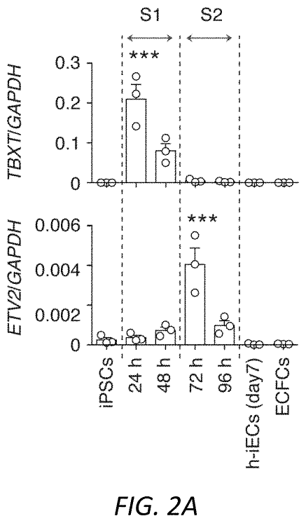

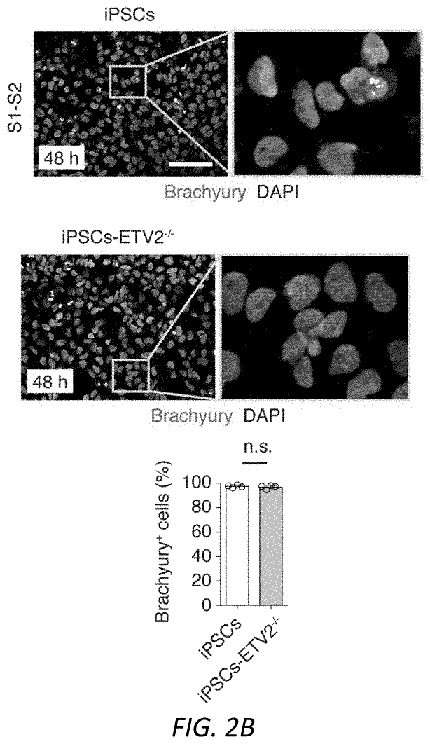

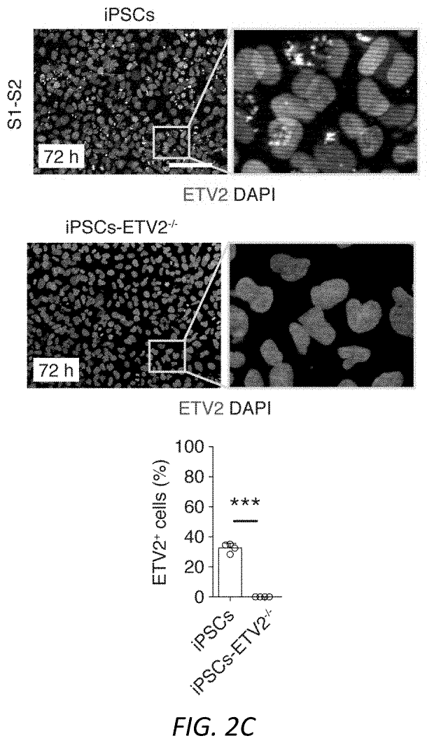

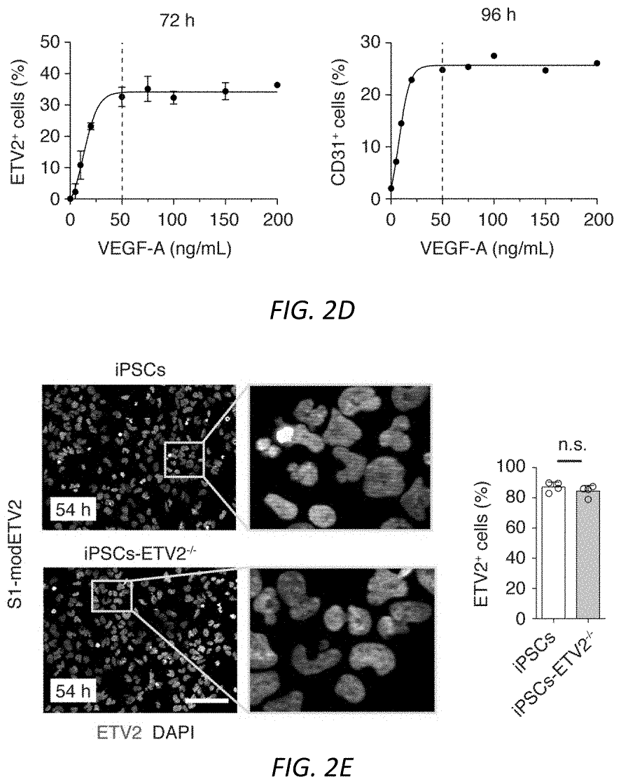

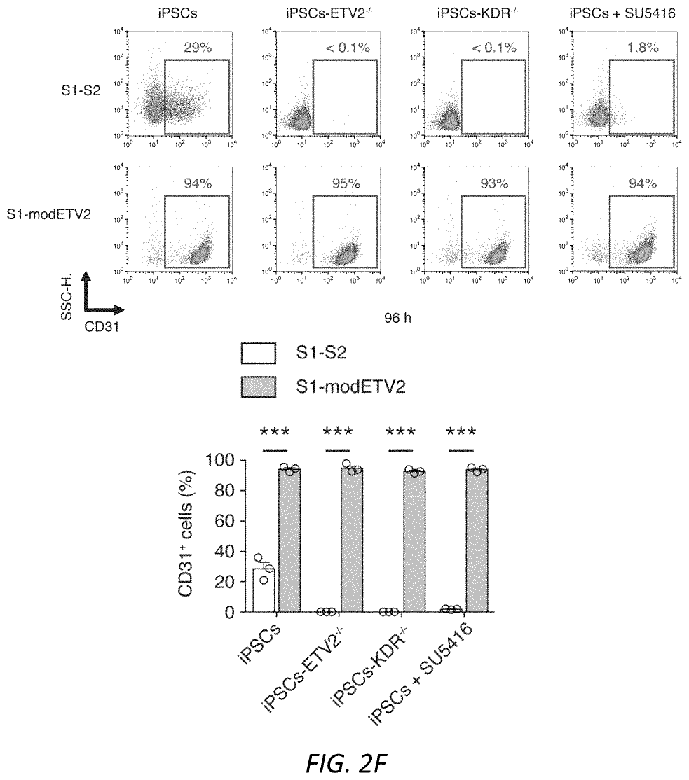

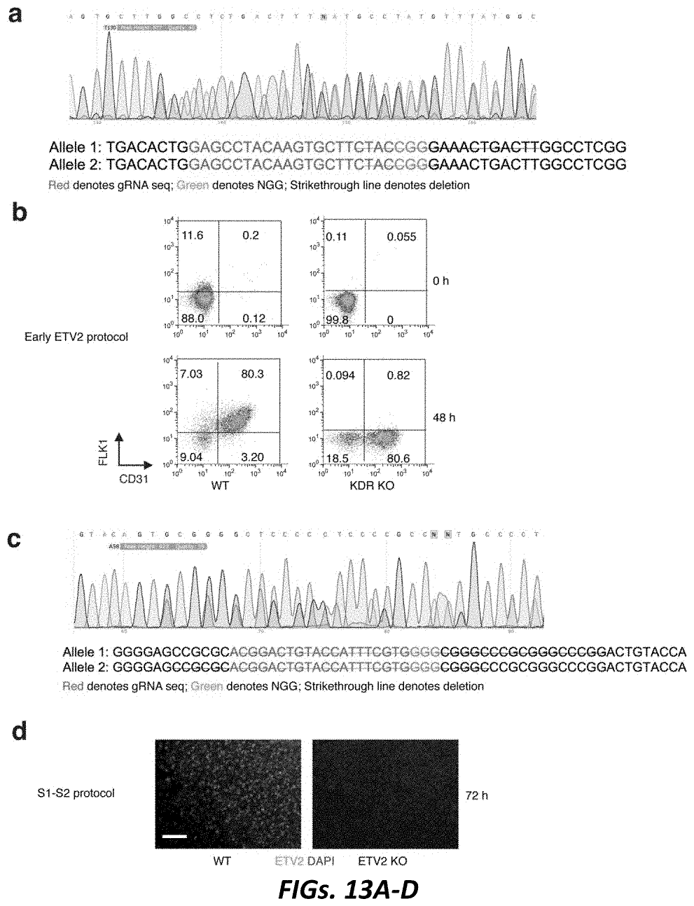

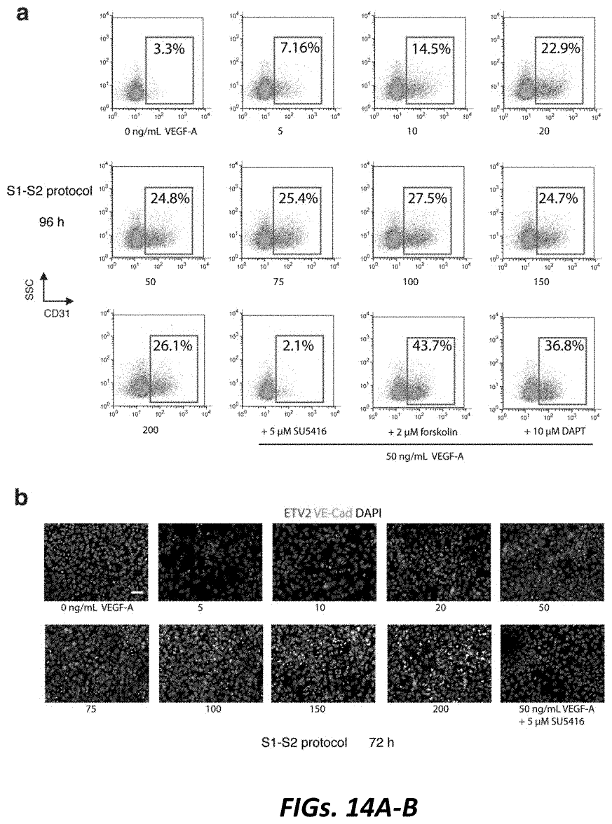

To further evaluate the issue of inefficiency, we carried out a transcriptional examination of the standard S1-52 differentiation protocol. As expected, conversion of h-iPSCs into h-MPCs coincided with transient activation of mesodermal transcription factors MIXL1 and TBXT (FIG. 2a; FIG. 12). Likewise, differentiation of h-MPCs into h-iECs involved activation of ETV2 (transiently) and then ERG (FIG. 2a; FIG. 12), consistent with previous vascular developmental descriptions^{9,10}. However, there were significant differences when comparing the efficiencies of transcription factor activation. On one hand, activation of TBXT (which encodes for Brachyury) was robust and highly uniform at 48 h (˜97% Brachyury+ cells; FIG. 2b), suggesting that the conversion of h-iPSCs into h-MPCs is unlikely to account for the large inefficiency observed in the S1-52 protocol. On the other hand, ETV2 activation by 72 h was limited and far from uniform (˜33% ETV2+ cells; FIG. 2c), indicating inefficient conversion of h-MPCs into h-iECs. Control h-iPSCs in which ETV2 was genetically abrogated using CRISPR-Cas9 (h-iPSC-ETV2^{-/-}) displayed unaltered TBXT activation and mesodermal conversion, but were unable to activate ETV2 and, in turn, incapable of initiating S2 (FIG. 2b-c; FIG. 13c-d). Because ETV2 expression is governed by VEGF signaling¹¹, we examined whether increasing the concentration of supplemented VEGF could improve its inefficient activation during S1-S2. However, we found that beyond 50 ng/mL VEGF failed to further increase the proportion of ETV2+ cells and thus the subsequent conversion to CD31+ h-iECs (FIG. 2d; FIG. 14).

Collectively, we found that while conversion of h-iPSCs into TBXT+ h-MPCs occurs very efficiently (>95%), activation of endogenous ETV2 in h-MPCs is clearly limited (˜30%) during the S1-S2 protocol and did not improve by simply increasing VEGF concentration. Thus, we concluded that in order to improve the conversion of h-iPSCs into h-iECs, emphasis should be put on finding new means to effectively activate ETV2 in the intermediate h-MPCs.

### Example 4. Transient Expression of Exogenous ETV2 Uniformly Convert h-MPCs into h-iECs

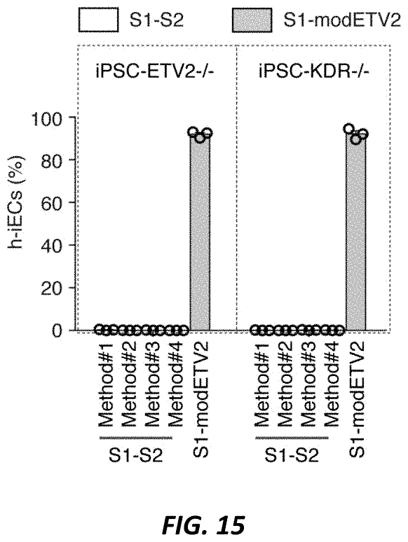

Our approach to more uniformly activate ETV2 in h-MPCs is to use modRNA. Indeed, 6 h after transfection of h-MPCs with modRNA(ETV2), >85% of the cells expressed ETV2 (FIG. 2e), a significant increase from the mere 30% observed in the S1-S2 protocol. Of note, h-iPSC-ETV2^{-/-} also displayed widespread ETV2 expression after transfection, indicating that activation was independent of endogenous ETV2 (FIG. 2e). This robust expression of ETV2 produced high rates of endothelial specification and efficient conversion into h-iECs in both unmodified h-iPSCs and h-iPSC-ETV2^{-/-} with the S1-modETV2 differentiation protocol (FIG. 2f). In contrast, with the S1-52 protocol, the differentiation process was less efficient and completely dependent on endogenous ETV2 expression (h-iPSC-ETV2^{-/-} failed to produce h-iECs) (FIG. 2f; FIG. 15). Moreover, the S1-modETV2 protocol maintains the differentiation process independent of VEGF signaling. Indeed, h-iPSCs in which KDR (which encodes for VEGFR-2) was genetically abrogated using CRISPR-Cas9 (h-iPSC-KDR^{-/-}) displayed an unaltered ability to differentiate into h-iECs (93% at 96 h) with the S1-modETV2 method but were incapable of differentiating (<0.1%) with the standard S1-S2 protocol (FIG. 2f; FIG. 13a-b; FIG. 15). Likewise, chemical abrogation of VEGFR2 signaling with the inhibitor SU5416 impaired the differentiation of h-iPSCs into h-iECs with the S1-S2 protocol but not with the S1-modETV2 protocol (<2% and 94% h-iECs at 96 h, respectively) (FIG. 2f).

Taken together, we showed that delivery of modRNA(ETV2) is an effective means to robustly and transiently express ETV2 in intermediate h-MPCs, which in turn initiates widespread conversion into h-iECs. Our S1-modETV2 protocol renders the differentiation process independent of VEGF signaling and of endogenous ETV2, thus overcoming one of the main limitations in current protocols.

### Example 5. Time of ETV2 Activation Affects the Transcriptional Profile of h-iECs

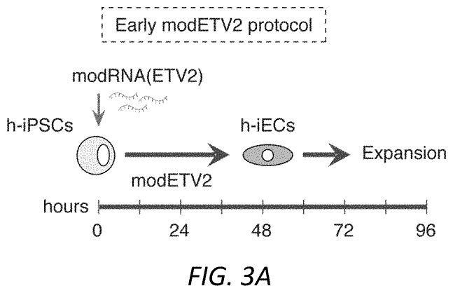

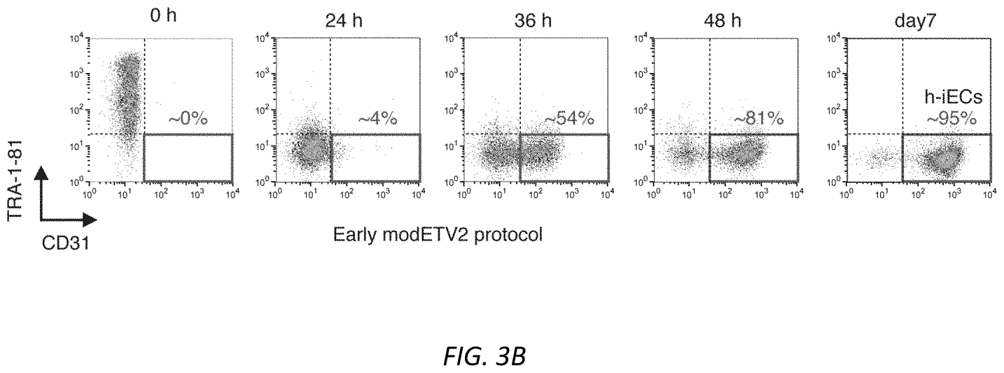

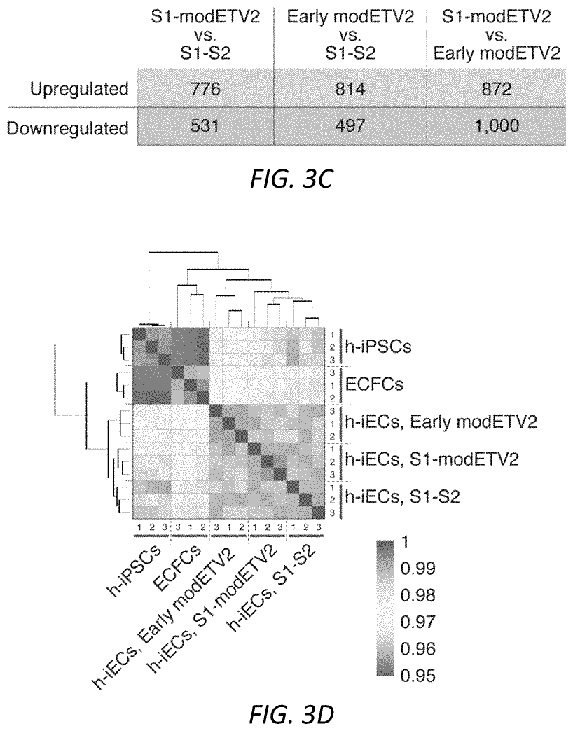

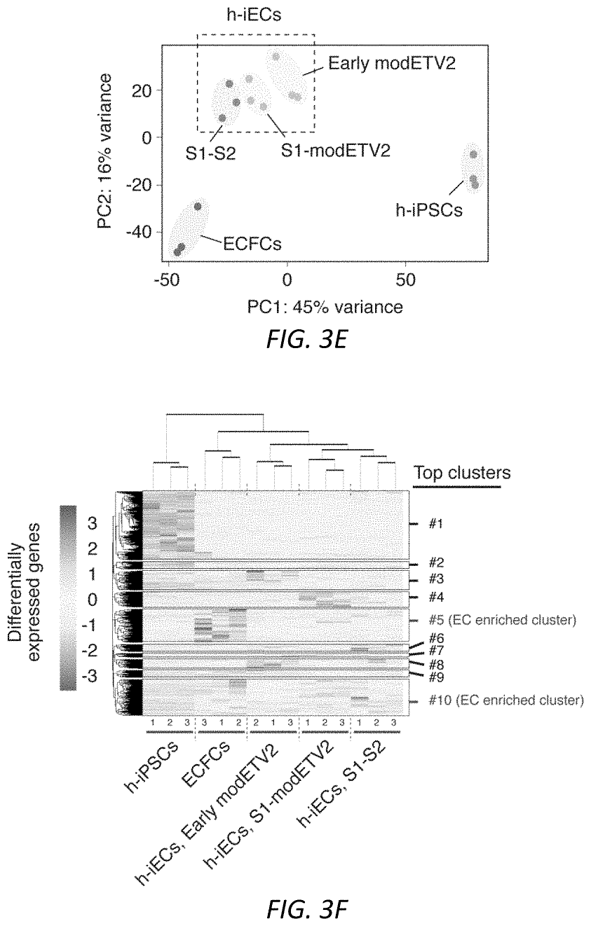

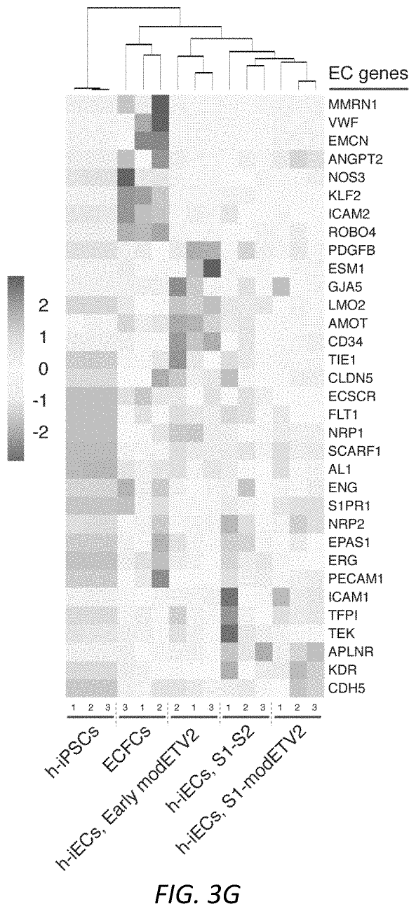

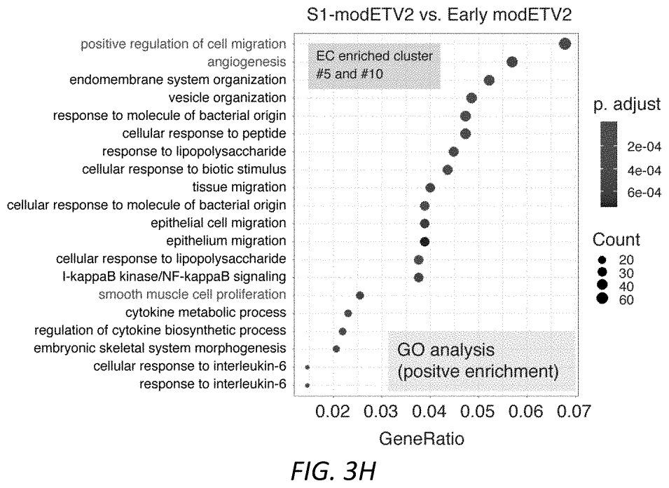

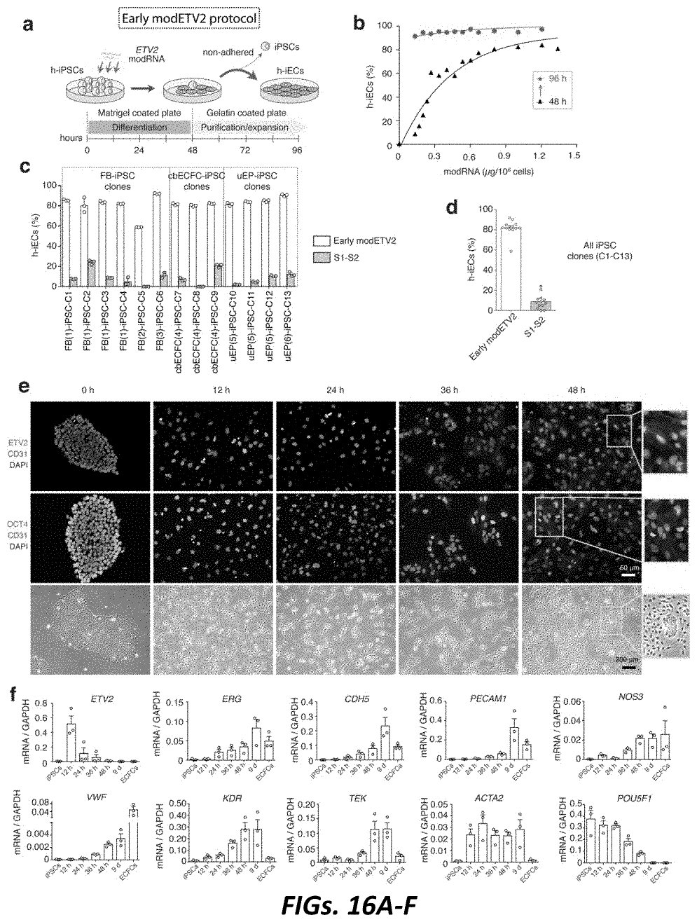

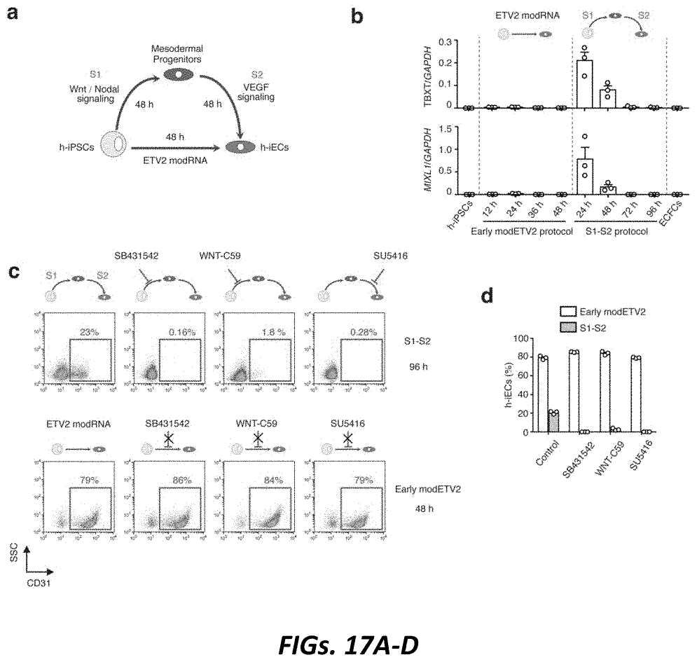

Previous studies have suggested that inducing ETV2 expression directly on h-iPSCs could generate h-iECs without transition through an intermediate mesodermal stage. However, it remains unclear whether this strategy produces functionally competent h-iECs. To address this question, we generated putative h-iECs by transfecting h-iPSCs with modRNA(ETV2) (protocol herein referred to as early modETV2) (FIG. 3a). This method converted human h-iPSCs into CD31+ cells rapidly and efficiently (FIG. 3b, FIG. 16a-b), which is consistent with previous reports¹³. Moreover, conversion was dependent on the concentration of modRNA(ETV2) and reproducible in all h-iPSCs clones tested, irrespective of the donor and cellular origin of the clones (FIG. 16c-d). Transfection of h-iPSCs with modRNA(ETV2) enabled early and transient expression of ETV2 (FIG. 16e-f), which occurred without previous significant expression of TBXT, suggesting a bypass of the intermediate mesodermal stage (FIG. 17A-D). With this approach, there was a remnant of undifferentiated (non-transfected) CD31−/OCT4+ cells at 48 h, which was deemed undesirable (FIG. 16e). Nonetheless, repeated subculture and purification of CD31+ cells largely mitigated this concern (FIG. 16f).

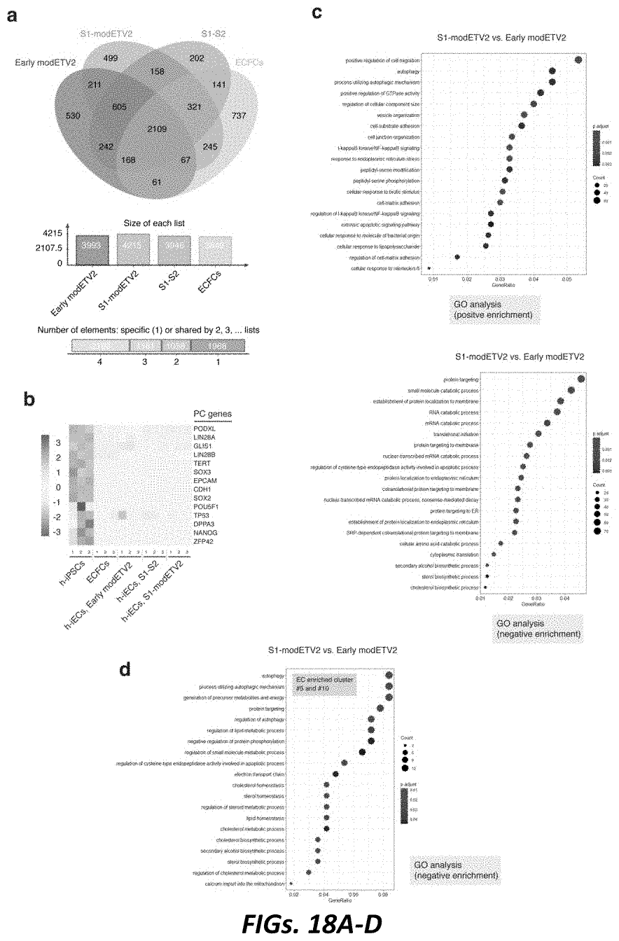

To further elucidate potential differences between h-iECs generated from our S1-modETV2, the S1-S2, and the early modETV2 protocols, we performed RNAseq analysis across multiple h-iECs samples generated from three independent h-iPSC lines using all three differentiation protocols. Human ECFCs and the parental undifferentiated h-iPSCs served as positive and negative controls, respectively. Globally, there were thousands of differentially expressed genes across all the h-iEC groups (FIG. 3c; FIG. 18a). Nevertheless, hierarchical clustering analysis of differentially expressed genes revealed 1) proximity between all the h-iEC groups, and 2) that h-iECs were transcriptionally closer to ECFCs than to h-iPSCs (FIG. 3f). These patterns of hierarchical association were confirmed by pairwise correlation (FIG. 3d) and principal component analyses (FIG. 3e). Moreover, analysis of selected endothelial and pluripotent genes confirmed that all groups of h-iECs were transcriptional more consistent with an endothelial phenotype than with the parental pluripotent state (FIG. 3g; FIG. 18b). Importantly, our analysis also revealed that among h-iECs, there was more transcriptional proximity between h-iECs generated from the standard S1-S2 protocol and our S1-modETV2 method (Pearson's correlation coefficient r=0.987) than between h-iECs derived from the early modETV2 protocol and the other h-iEC groups (FIG. 3d).

To gain more insight into the transcriptional differences, we carried out gene ontology (GO) enrichment analysis between h-iECs generated with our S1-modETV2 and the early modETV2 differentiation protocols. Of note, analysis of all differentially expressed genes revealed that h-iECs generated with our S1-modETV2 displayed significant enrichment in genes associated with positive regulation of cell migration (FIG. 18c). Moreover, a GO analysis was performed with differentially expressed genes from EC clusters #5 and #10 (cluster elucidated by hierarchical clustering analysis; FIG. 3f). Results indicated positive enrichment in h-iECs generated with our S1-modETV2 of genes associated with not only cell migration but also angiogenesis and smooth muscle proliferation (FIG. 3h; FIG. 18d), suggesting differences in genes affecting critical vascular function.

### Example 6. Early Activation of ETV2 Renders Putative h-iECs with Impaired Functionality

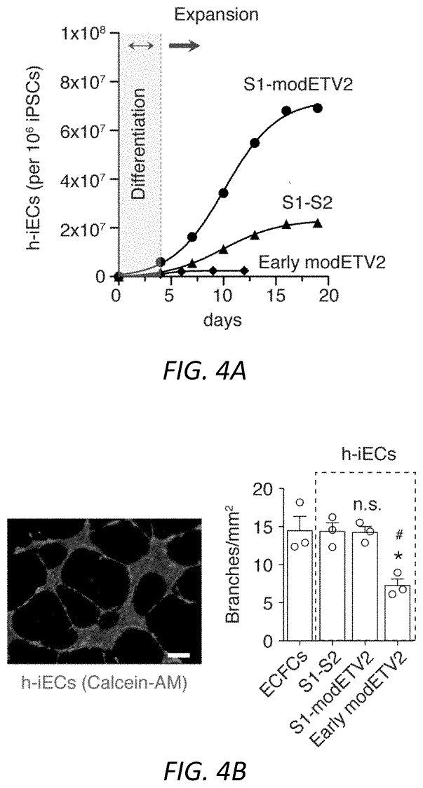

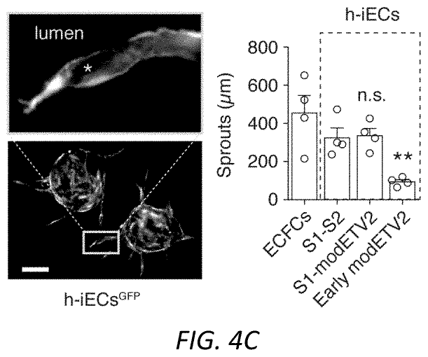

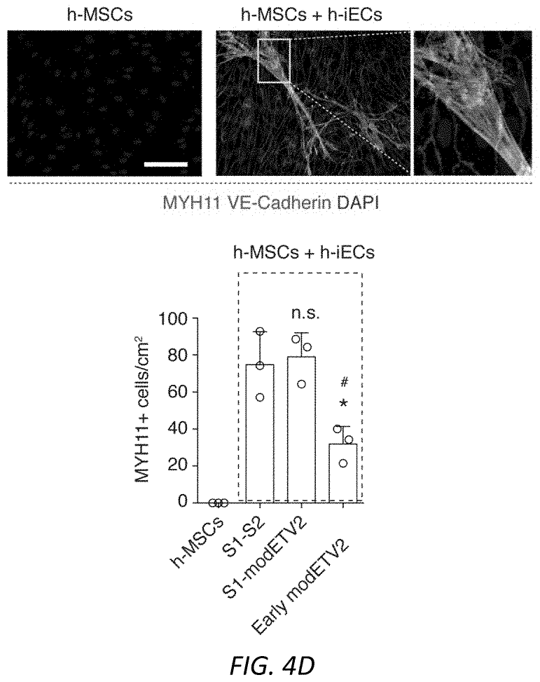

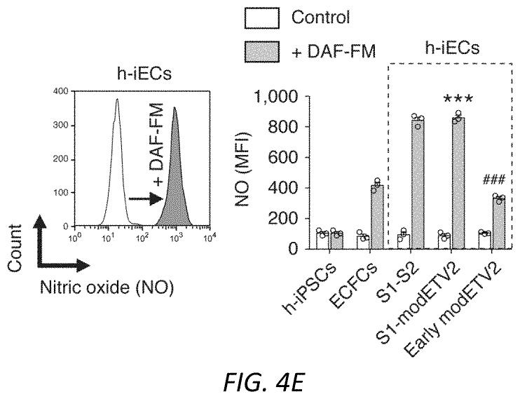

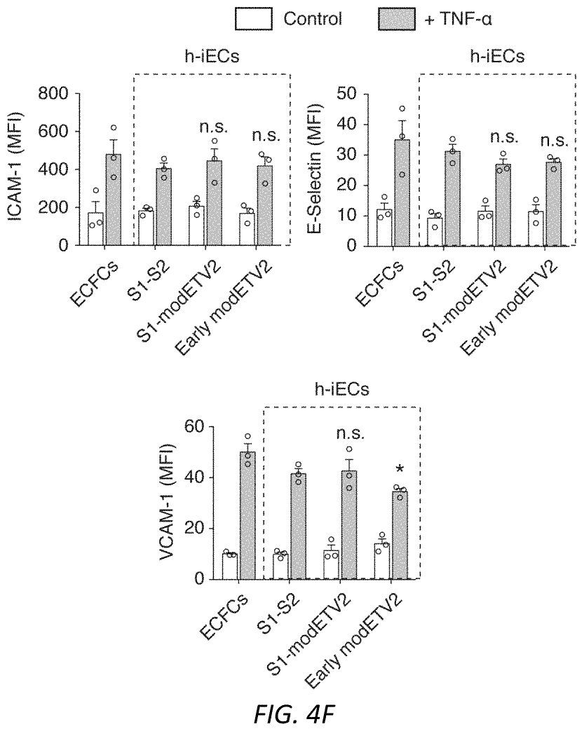

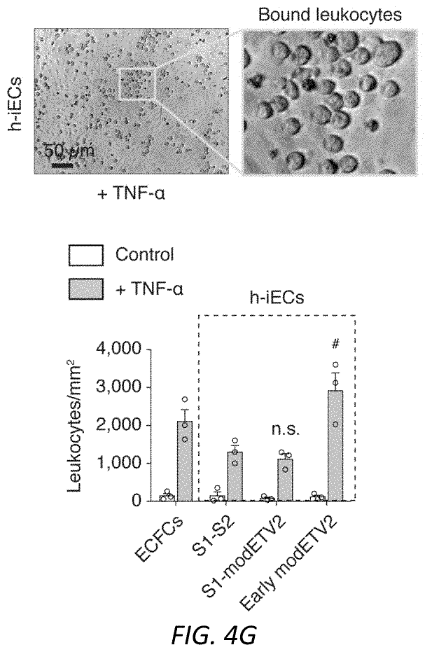

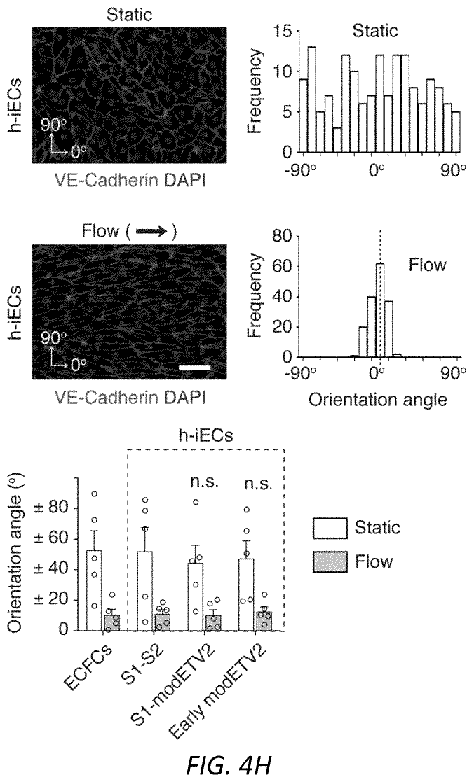

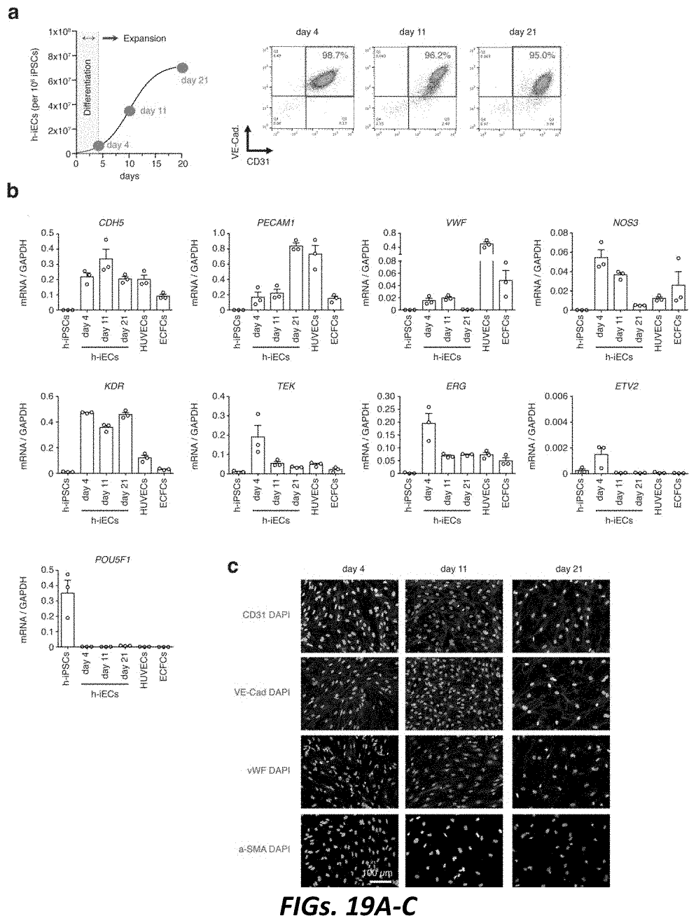

Next, we examined whether the transcriptional differences observed between h-iECs generated from different protocols affected their capacity to function as proper ECs. Specifically, we compared h-iECs that were generated with the standard S1-52, our S1-modETV2, and the early modETV2 protocols. Of note, ETV2 expression is transient in both differentiation protocols, and thus at the time of h-iEC characterization, ETV2 expression was completely absent (FIG. 1d). Human cord blood-derived ECFCs served as control for bona fide ECs. First, we assessed the capacity to grow in culture. Previous studies have shown mixed results with regards to the expansion potential of h-iECs and currently there is no consensus on this issue¹⁴. We observed that h-iECs generated with our S1-modETV2 protocol were easily expanded in culture for a period of 3 weeks, with an average expansion yield of ˜70-fold (FIG. 4a). This yield was significantly higher than that of h-iECs produced with the S1-2 differentiation protocol (˜20-fold), which was mainly attributed to differences in efficiency during the initial 4 days of differentiation (FIG. 4a). More striking, however, was the lack of expansion displayed by h-iECs generated with the early modETV2 protocol. Notwithstanding the high differentiation efficiency of this method (FIG. 3a), these putative h-iECs ceased proliferating after approximately two weeks in culture with only a modest overall yield of ˜2-fold (FIG. 4a). In addition, it is important to note that h-iECs obtained by the S1-modETV2 method retained an endothelial phenotype along their expansion in culture. Examination at days 4, 11, and 21 during expansion revealed that h-iECs remained fairly pure (>95% VE-cadherin+/CD31+ cells), maintained expression of EC markers at the mRNA and protein levels, and remained negative for POU5F1 (OCT4) and α-Smooth muscle actin (α-SMA. (FIG. 19A-C).

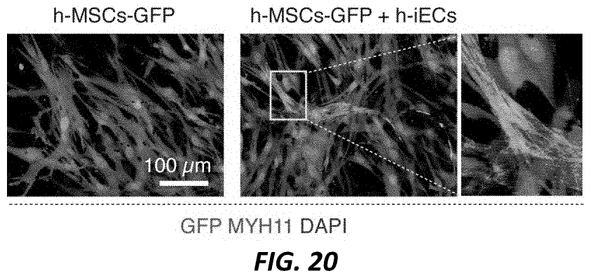

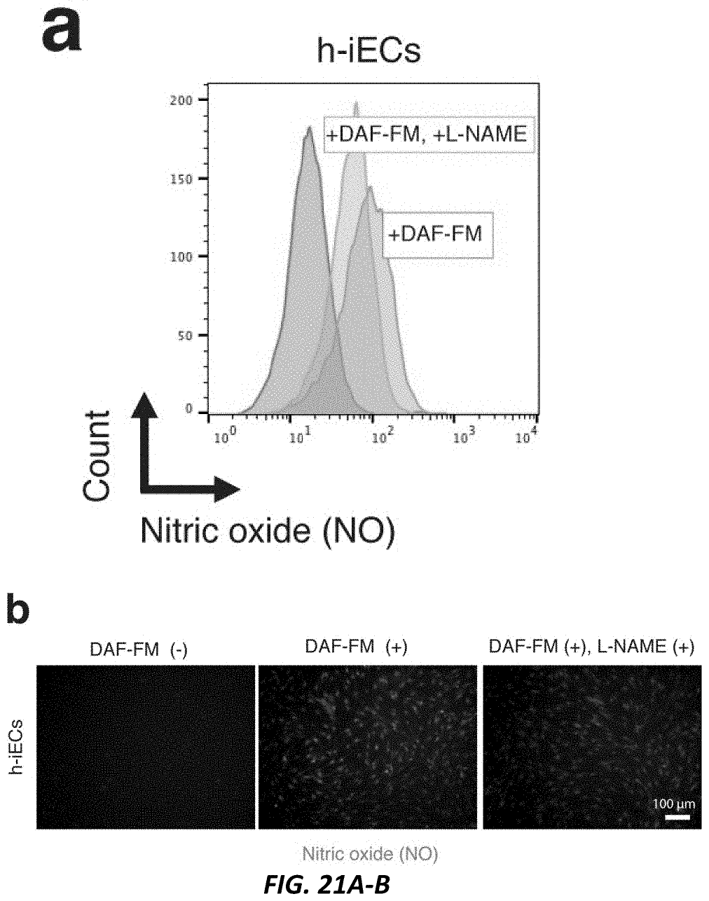

We then evaluated the performance of h-iECs using an array of standard endothelial functional assays, including ability to: (i) assemble into capillary-like structures (FIG. 4b); (ii) launch angiogenic sprouts with proper lumens (FIG. 4c); (iii) induce smooth muscle differentiation of human mesenchymal stem cells (h-MSCs) (FIG. 4d; FIG. 20); (iv) produce nitric oxide (NO) (FIG. 4e; FIG. 21A-B); (v) up-regulate expression of leukocyte adhesion molecules (E-selectin, ICAM-1, and VCAM-1) upon exposure to tumor necrosis factor-alpha (TNF-α) (FIG. 4f); (vi) up-regulate leukocyte binding upon exposure to TNF-α (FIG. 4g); and (vii) sense and adapt to shear flow, aligning to the direction of flow (FIG. 4h). Collectively, this comprehensive examination confirmed that h-iECs generated with both the S1-S2 and our S1-modETV2 differentiation protocols were functionally very similar, with no statistically significant differences between both groups in any of the assays (FIG. 4b-h). In addition, both h-iEC groups were comparable to the control ECFCs (the only exception was higher NO production by h-iECs; P<0.001; FIG. 4e), thus suggesting adequate endothelial function. In contrast, h-iECs generated with the early modETV2 protocol showed signs of impaired functionality. When compared to the control ECFCs, these h-iECs appeared competent in some fundamental capacities such as the ability to regulate leukocyte adhesion upon an inflammatory stimulus, and the capacity to align in the direction of flow. However, h-iECs generated with the early modETV2 protocol displayed quantitative deficiencies in several important respects, including a significantly lower ability to assemble into capillary-like structures (P<0.05; FIG. 4b), to launch proper angiogenic sprouts (P<0.01; FIG. 4c), and to induce smooth muscle differentiation of h-MSCs (P<0.05; FIG. 4d). These differences were also statistically significant when compared to h-iECs generated with both the S1-S2 and our S1-modETV2 differentiation protocols, suggesting certain fundamental phenotypic differences between h-iECs generated from the different protocols.

### Example 7. Timely Activation of ETV2 is Critical for Proper Vascular Network-Forming Ability

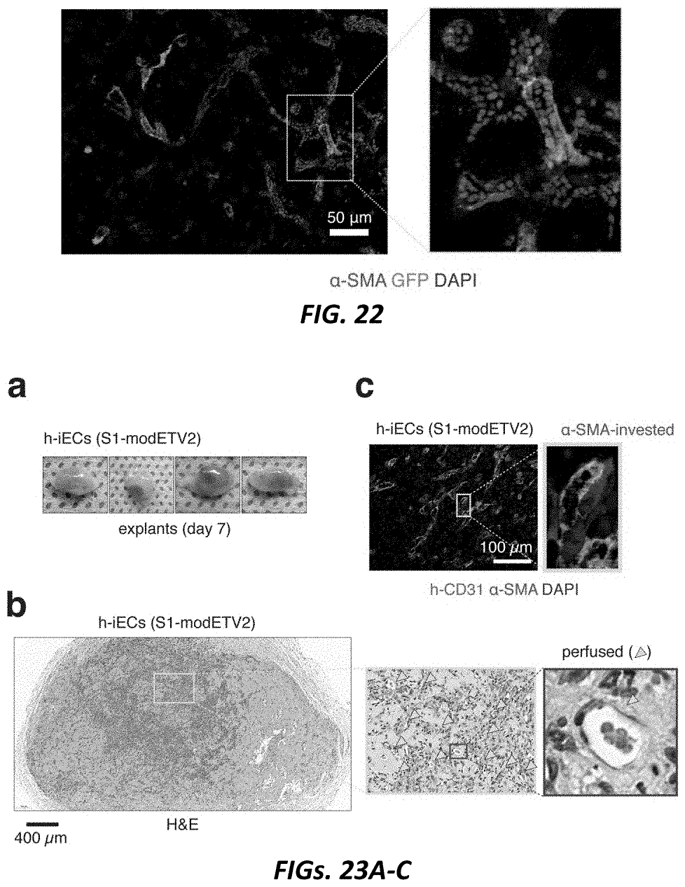

Lastly, we examined the capacity of the different h-iECs to assemble into functional blood vessels in vivo (FIG. 5). To this end, we used our model of vascular network formation in which human ECs are combined with supporting MSCs in a hydrogel, and the grafts are then implanted into immunodeficient SCID mice¹⁵. After 7 days in vivo, macroscopic examination of the explants suggested differences in the degree of vascularization between implants containing different types of h-iECs (n=5) (FIG. 5a). Histological (H&E) analysis revealed that grafts with h-iECs generated with the S1-modETV2 protocol had an extensive network of perfused microvessels (FIG. 5b, left). These microvessels were primarily lined by the h-iECs, as confirmed by the expression of human-specific CD31 (FIG. 5e, left), by the affinity for UEA-1 (FIG. 5d, left), and by the use of gfp-labeled h-iECs (FIG. 22). Moreover, these human lumens contained mouse erythrocytes (FIG. 5b, left), indicating formation of functional anastomoses with the host circulatory system. In contrast, the number of perfused human vessels in grafts with h-iECs generated with the early modETV2 protocol was exceedingly low (FIG. 5b, right). These h-iECs remained organized as lumenal structures (FIG. 5e,d, right), but the lumens were rarely perfused (FIG. 5b, right). Indeed, microvessel density in grafts containing h-iECs from the early modETV2 protocol was significantly lower than in any other group (FIG. 5c). Of note, there were no significant differences in vessel density between grafts formed with h-iECs from the S1-modETV2 protocol, the S1-S2 protocol (both of which underwent transition through mesodermal intermediates) and the control ECFCs.

It is important to note that although co-transplantation with MSCs facilitates engraftment, h-iECs derived from the S1-modETV2 protocol were also able to engraft and form perfused vessels when implanted alone, without MSCs (FIG. 23A-C). Indeed, grafts containing h-iECs alone became vascularized in 7 days and histological analysis confirmed the presence of numerous microvessels lined by the h-iECs (FIG. 23A-C).

We also examined the presence of mural cell investment around the newly-formed human vessels, a hallmark of proper vessel maturation and stabilization¹⁶. There was a striking difference between h-iECs generated with the S1-modETV2 protocol and those generated with the early modETV2 with regard to perivascular investment (FIG. 5d-f). In grafts with h-iECs from the S1-modETV2 protocol, the large majority (˜87%) of the human vessels had proper coverage by perivascular cells expressing α-smooth muscle actin (α-SMA) (FIG. 5d-e, left; FIG. 5f). This high percentage of perivascular coverage is to be expected by day 7 in this model¹⁷, and vessels formed by control ECFCs consistently displayed high coverage (FIG. 5f). In contrast, grafts containing h-iECs from the early modETV2 protocol had only ˜8% of their human vessels covered by α-SMA+ cells (FIG. 5d-e, right; FIG. 5f). These h-iECs were able to engraft and self-assemble into recognizable lumenal structures, but these structures lacked perivascular cells indicating inadequate maturation. Moreover, TUNEL staining of explants at day 7 revealed signs of apoptosis in a large percentage of human vessels lined by h-iECs from the early modETV2 protocol (FIG. 5g), an indication of vessel instability. On the other hand, 30 days after implantation, grafts that used h-iECs generated with the S1-modETV2 protocol still contained extensive and uniform networks of human vessels with proper perivascular coverage (FIG. 5h).

Taken all together, we demonstrated that during the differentiation of h-iPSCs into h-iECs, the ETV2 activation stage is critical. With our optimized S1-modETV2 protocol, activation of ETV2 occurred at the intermediate mesodermal stage, which produced h-iECs that were phenotypically and functionally competent. In contrast, bypassing transition through the mesodermal stage by early activation of ETV2 produced putative h-iECs with a transcriptional profile further away from that of bona fide ECs, and, more importantly, with impaired functionality.

### Example 8. Bioengineering Hemophilia a Patient-Specific Vascular Networks that Express High Levels of Full-Length Coagulation Factor VIII

Over the last few decades, Hemophilia A has been a particularly appealing target for gene therapy and a plethora of approaches have been proposed with various degrees of pre-clinical and clinical success²⁵. Most efforts have focused on direct in vivo gene therapy with the use of viral vectors, including AAV vectors. However, notwithstanding the remarkable progress achieved in this field thus far, most in vivo gene therapy approaches for hemophilia A remain limited by a number of challenges that hamper their clinical translation.

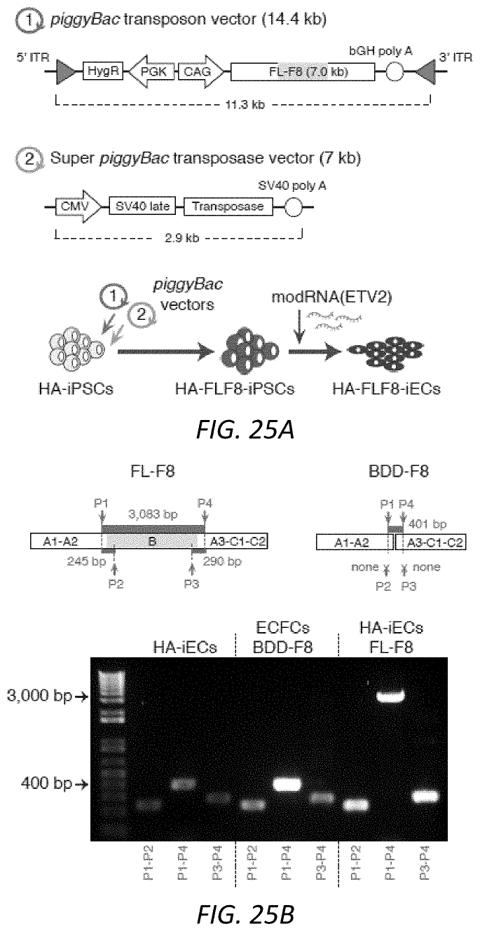

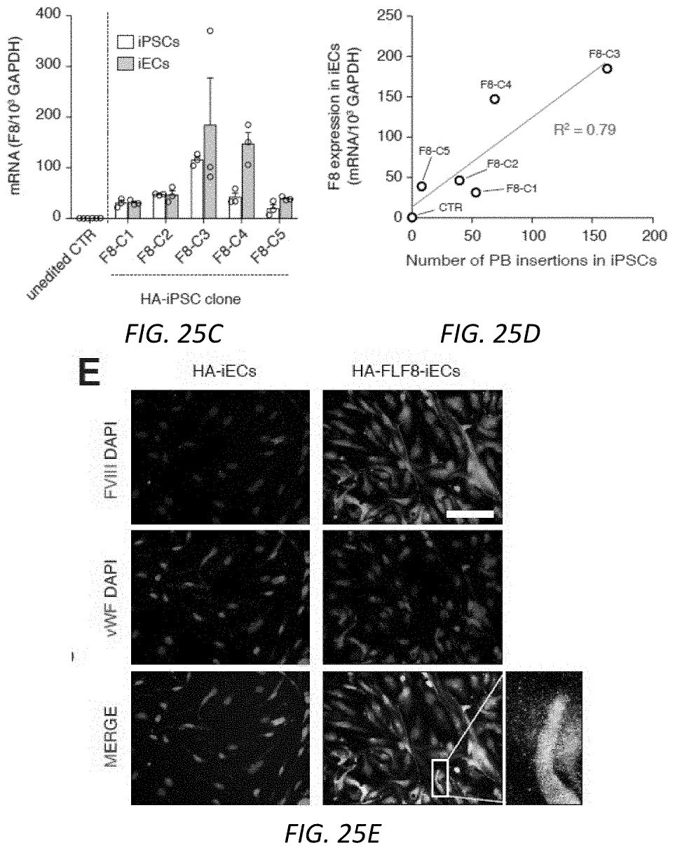

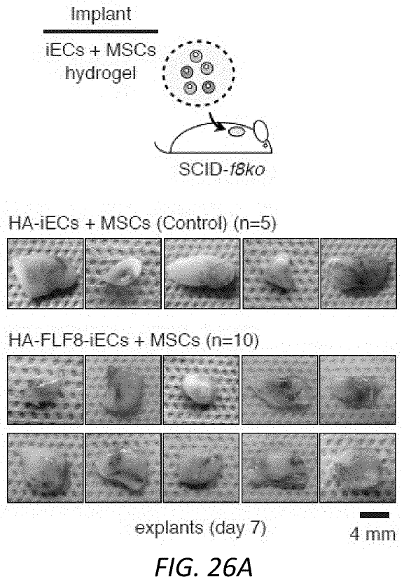

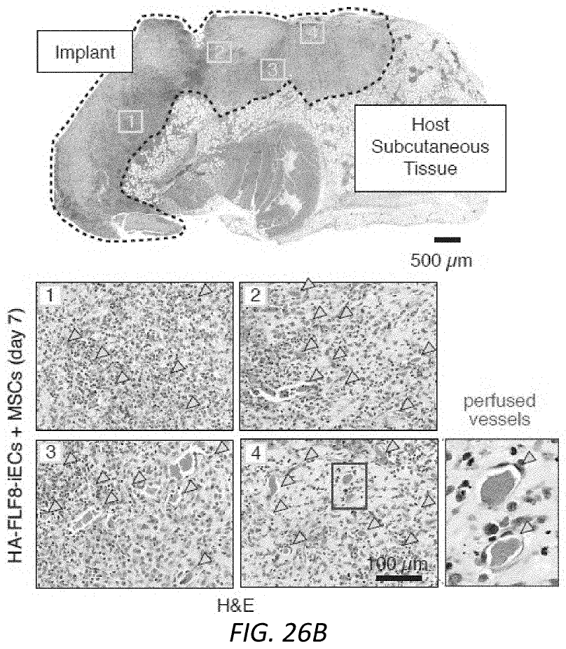

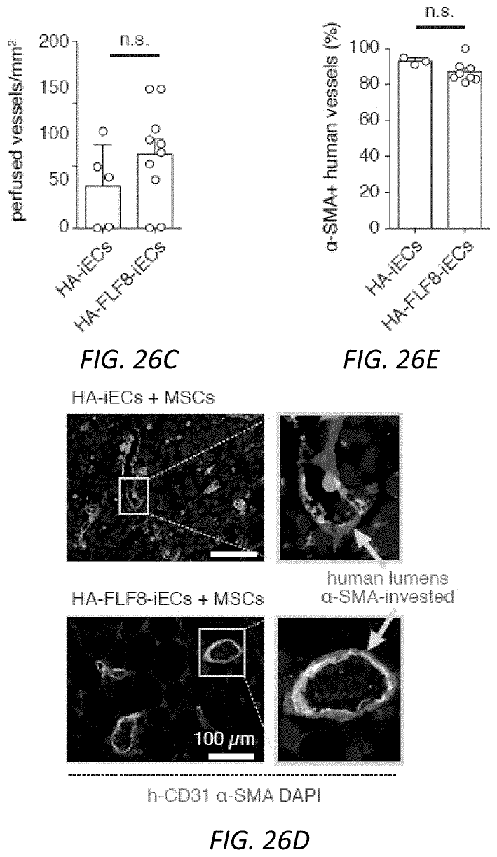

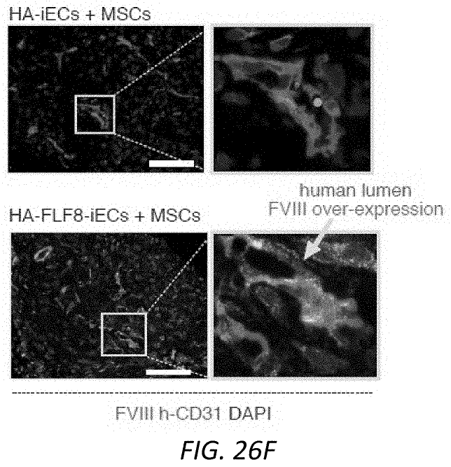

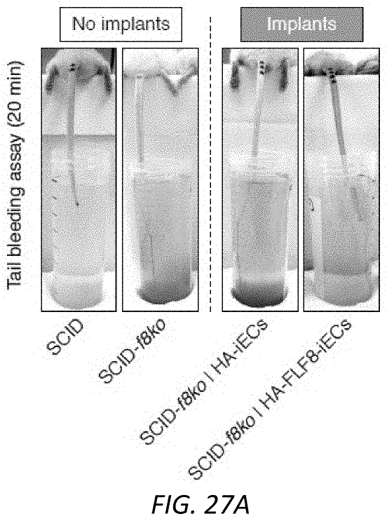

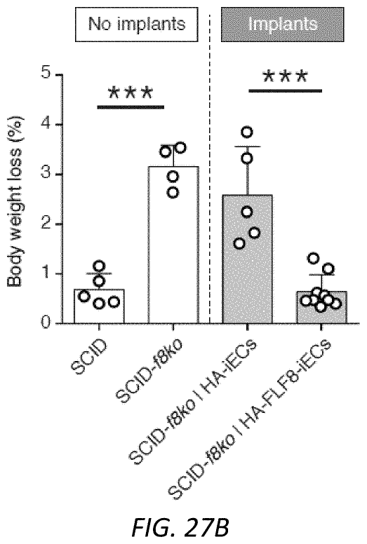

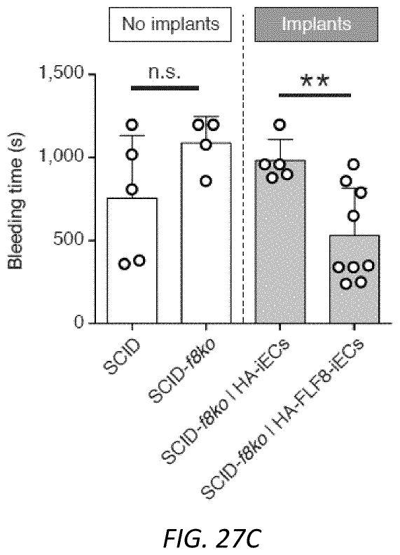

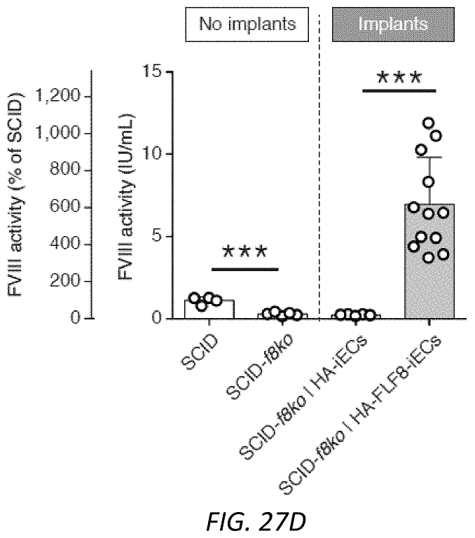

Described herein is an alternative to current hemophilia A treatments that is non-viral, scalable, autologous, and reversible. Although Hemophilia A is a primary disease focus, the massive insertion capacity of the piggyBac gene engineering platform described herein allows more flexibility when coupled with bioengineered vascular implants. The differentiation of HA-iPSCs into HA-iECs was carried out with high efficiency across all patients and independently of the HA-iPSC clones selected. Importantly, the HA-iECs could be expanded in culture with ease to generate the necessary cells for our grafts. As mentioned earlier, the usage of HA-iECs was deliberate and the reasons twofold: 1) ECs are the natural producers of FVIII in the body. Thus, ECs contain the appropriate cellular machinery to package FVIII with von Willebrand factor (vWF) into Weibel-Palade bodies and to carry out an effective secretion, activation, and protection of FVIII once in the blood plasma²¹. Indeed, we showed that upon transduction, overexpressed FVIII partially co-localized with vWF in the modified HA-FLF8-iECs (FIG. 25E); and 2) ECs line the lumen of the vasculature and thus should in principle allows for direct secretion of FVIII into the bloodstream. This was apparent in our subcutaneous grafts, where the presence of lumenal structures that overexpressed FVIII was evident as well as by the detection of human FVIII in the plasma of implant-bearing mice (FIG. 26F, FIG. 27D).

A second important focus of our study was avoiding the use of viral vectors. Once more, the reasons were twofold: 1) to eliminate adverse immunological reactions; and 2) to circumvent the limitation imposed by a restricted viral cargo size; avoiding size restriction would, in turn, open up the possibility of transducing cells with the full-length version of the F8 gene. With this in mind, we implemented a non-viral piggyBac DNA transposon strategy to genetically engineer patients' HA-iPSCs for FVIII overexpression. Unlike most viral vectors, piggyBac vectors can insert large genetic cargos, reportedly up to 100 kb (˜9.1 kb without a loss of efficiency) 20, and thus we were not limited by cargo size. Indeed, by using the piggyBac transposon system, not only we were able to encode for the full-length version of the human F8 gene, but also, we were able to insert multiple copies (ranging from 8-160), which highlights one of the most notable advantages of the piggyBac system (FIG. 25D). Moreover, we showed that there was a linear relationship between piggyBac transposon insert number and gene-expression, and although we did not optimize the system for maximal insertion, future studies could investigate a potential ceiling for the number of FLF8 inserts before loss of cell function or cytotoxicity may occur. Additionally, despite the semi-random nature of the piggyBac transposon insertions, one could envision that working with iPSCs allows genetically screening for clones with high levels of insertion exclusively into non-coding regions of the DNA, thereby minimizing the potential oncogenicity of the cells. Genomic methods of mapping piggyBac insertion have been previously outlined and thorough characterization will be necessary to guarantee the long-term safety of implants in future clinical applications 29 It is also important to note that all the genetic modifications were done at the iPSC level, prior to their differentiation into HA-iECs. This allowed us to easily select for clones of HA-FLF8-iPSCs with high insertion numbers and thus high levels of F8 expression. Furthermore, we showed that overexpression of F8 in HA-iPSCs did not affect subsequent differentiation—the resulting HA-FLF8-iECs displayed high levels of gene expression and production of FVIII, similarly to the parental HA-FLF8-iPSCs. Collectively, the use of the piggyBac transposon system was extremely instrumental for our study. Previously, a pre-clinical study by Matsui et. al. (2014) used a piggyBac vector that encoded FLF8 in a murine model of in vivo gene therapy²⁰. However, to our knowledge, this is the first time that the piggyBac system has been used in the context of ex vivo gene therapy and that resulted in successful overexpression of full-length FVIII in hemophilia A patients' ECs.

A third focus of our study was related to the engraftment of genetically-engineered HA-FLF8-iECs, with special considerations to accessibility and the overall reversibility potential of the treatment. For years, our group has worked intensively on the question of engraftment, and our solution entails combining ECs with supporting stromal cells (i.e., MSCs) into a suitable collagen-based hydrogel³⁰⁻³² In this configuration, upon subcutaneous transplantation of the grafts, HA-FLF8-iECs are able to self-assemble into a vascular network that forms anastomoses and connects with the host circulatory system. This mode of engraftment, in turn, allows the implanted ECs to rapidly adopt a proper physiological role, lining the lumen of perfused vessels, which facilitates integration with the host. Moreover, we previously demonstrated that this mode of engraftment results in tight cellular confinement, which reduces potential safety concerns and allows effective monitoring and reversibility by a simple implant excision³³. Alternative modes of EC engraftment have been proposed. For example, Xu et al., (2009) injected normal (non-hemophilic) murine iPSC-derived ECs into the liver of hemophilic mice, correcting their bleeding deficiency³⁴. However, although the cells were injected into the liver, higher levels of FVIII mRNA were detected in spleen, heart, and kidney tissues of injected animals, suggesting widespread dissemination and thus complicating accessibility and reversibility.

There are other ex vivo gene therapy studies that use ECs and that are of significant importance in the field. However, the majority of these studies used viral vectors, and none produced full-length FVIII, which are two distinctive features of our approach. For example, a recent study by Olgasi et al., (2018) used a lentiviral vector to genetically modified patients' HA-iPSC-derived ECs to express BDD-FVIII³⁵. The modified cells were then transplanted either via portal vein or intraperitoneally, where they engrafted and in turn were able to correct the coagulation deficiency in the recipient animals. Engrafting cells in the liver, however, compromises confinement and the possibility of graft retrieval. In another study, Ozelo et al., (2014) isolated blood outgrowth ECs (BOECs) from hemophilic dogs and genetically modified them to express BDD-FVIII via lentiviral vectors³⁶. In this case, the modified BOECs were embedded into fibrin gels, which facilitated confinement. This study showed exceptional potential upon surgical implantation of the grafts into the omentum of the dogs; nevertheless, concerns around the use of viral vectors and a truncated version of FVIII remains. A study by Park et al. (2015) utilized CRISPR technology to correct the FVIII inversion in patient iPSCs followed by endothelial differentiation and subsequent injection for correction of the disease phenotype³⁷. Although, these cells were injected into the hindlimb without confinement, where engraftment of endothelial cells and the connection to the host bloodstream are unclear.

Previous ex vivo gene therapy studies have also included the use of alternative non-endothelial cells as gene delivery vehicles, including hemopoietic stem cells (HSCs)³⁸⁻⁴⁰. For example, Shi et al., (2014) transduced human cord blood-derived CD34+ HSCs with a lentiviral construct in which the human platelet glycoprotein IIb gene promoter (αIIb^{pr}) was used to direct megakaryocyte-specific synthesis of human BDD-F8⁴⁰. Upon transplantation into irradiated recipients, the modified HSCs engrafted and created blood cell chimerism, including human megakaryocytes that produced BDD-FVIII-containing platelets. This platelet gene therapy was shown to correct bleeding deficiency in immunocompromised hemophilia A mice. The use of HSCs is particularly appealing in several respects. First, long-term engraftment of HSCs is feasible, and the process is reasonably well understood; and second, once HSCs engraft, in principle they can permanently replenish the cells producing the FVIII-containing platelets. Additionally, our piggyBac transposon approach to inserting full-length F8 offers advantages in scalability. Other EC ex-vivo approaches, due to a singular gene correction 37 or low lentiviral insertion number of BDD-F8, report restoring mice to a non-severe hemophilia pathology at 6-30% healthy levels of FVIII in mouse models (EC sources above). Instead, our platform can insert up to 160 copies of full-length F8 cDNA, allowing us to raise circulating protein levels up to 1,300% of the phenotypic level of a healthy mouse.

Materials and Methods for Example 8

The following materials and methods were used for Example 8.

Isolation and Culture of Human Urine-Derived Epithelial Cells

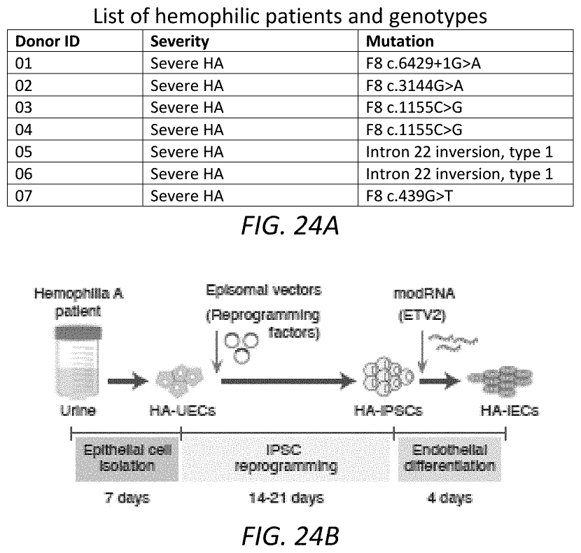

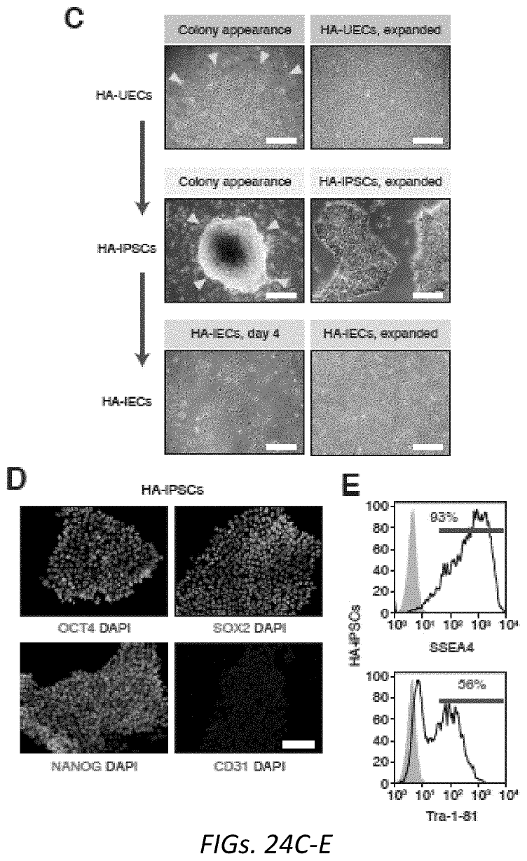

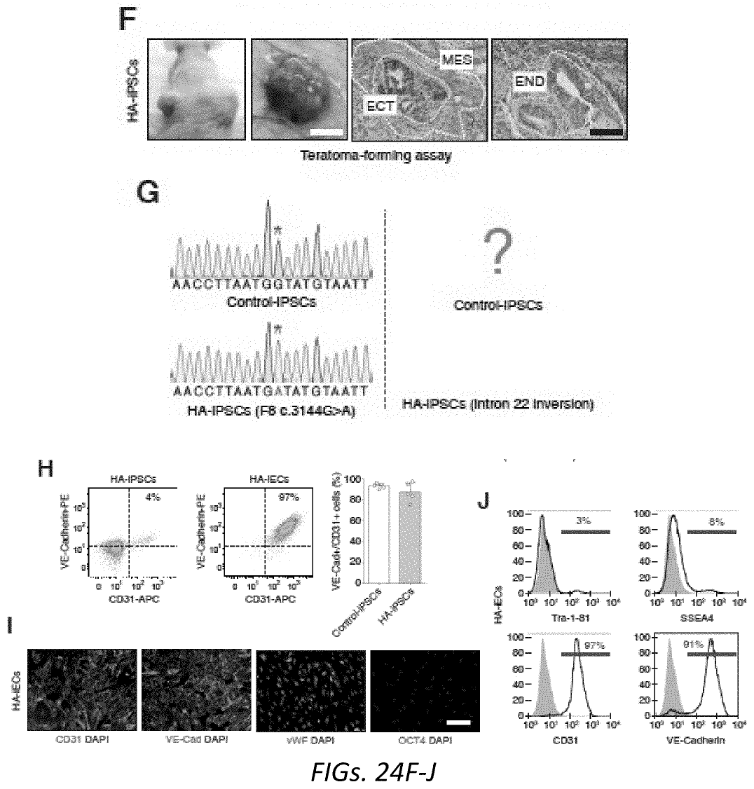

De-identified urine samples were obtained from patients with severe hemophilia A and from healthy individuals in accordance with Institutional Review Board-approved protocols at Boston Children's Hospital. Informed consent was obtained from all donors. The list of hemophilic patients with their corresponding mutant genotype is in FIG. 24A. Urine samples (˜100 mL) from hemophilia patients and healthy individuals were collected in sterile containers and kept on ice. Urine samples were then transferred into 50 ml tubes inside a tissue culture hood and these tubes were centrifuged at 400 g for 10 minutes at room temperature. The supernatant was carefully discarded. Pellets were washed by PBS twice before resuspension in endothelial growth medium: EGM-2 (except for hydrocortisone; PromoCell, Cat No. C22111) supplemented with 10% FBS (Atlanta Biologicals, Cat No. S11595), and 1×PSG (Gibco, Cat No. 10378016). Cells collected from each sample were cultured separately in 1% gelatin-coated 6 well plates for 14 days. Medium was changed every two days. Visible cell colonies appeared routinely after 5-7 days, typically an average of 5-10 per sample, showing typical epithelial morphology. Urine-derived epithelial cells (UECs) were then split onto a bigger surface aided by TrypLE select enzyme (ThermoFisher, Cat No. 12563029) when the culture grew confluent. All experiments were carried out with UECs up to passage 4.

Isolation and Culture of Human MSCs and ECs

Human MSCs (h-MSCs) were isolated from white adipose tissue as previously described¹. h-MSCs were cultured on uncoated plates using MSC-medium: MSCGM (Lonza, Cat No. PT-3001) supplemented with 10% GenClone FBS (Genesee, Cat No. 25-514), and 1× penicillin-streptomycin-glutamine (PSG, ThermoFisher, Cat No. 10378106). All experiments were carried out with h-MSCs between passages 6-10. Control ECs were isolated from cord blood as previously described¹ and grown in EGM-2 on 1% gelatin-coated plates.

Generation and Culture of Human HA-iPSCs

Human urine-derived epithelial cells from patient #1 (genotype F8 c.6429+1G>A; Table A), #5, and #6 (both with intron 22 inversion, type 1) were used to generate Human hemophilia A patient induced pluripotent stem cells (HA-iPSCs) via non-integrating episomal expression of selected reprogramming factors 2. Briefly, four plasmids encoding hOCT4, hSOX2, hKLF4, hL-MYC, hLIN-28, and EBNA-1 (Addgene plasmids #27077, #27078, #27080, and #37624 deposited by Shinya Yamanaka) were introduced via electroporation into HA-UECs. Transfected cells were then cultured with TeSR-E7 medium (STEMCELL, Cat No. 05910). HA-iPSC colonies spontaneously emerged between days 15-25. Colonies were then transferred to a Matrigel-coated (Corning, Cat No. 354277), feeder-free culture plate for expansion and were routinely checked for absence of mycoplasma using a PCR Mycoplasma Detection Kit (abm, Cat No. G238). HA-iPSCs were cultured in mTeSR1 medium (STEMCELL, Cat No. 85850) on 6-well plates coated with Matrigel. At 80% confluency, h-iPSCs were detached using ReLeSR reagent (STEMCELL, Cat No. 05872), split at 1:6 ratio, and plated in media supplemented with 10 μM Y27632 (Selleckchem, Cat No. S1049).Culture medium was changed daily. The iPSC phenotype was validated by expression of pluripotent transcription factors OCT4, NANOG, and SOX2; and by the ability to form teratomas. A teratoma formation assay was performed by injecting million h-iPSCs mixed in 100 μL Matrigel into the dorsal flank of nude mice Four weeks after the injection, tumors were surgically dissected from the mice, weighed, fixed in formalin, and embedded in paraffin for histology. Sections were stained with hematoxylin and eosin (H&E). Antibody information is detailed in Table 12.

TABLE 12
List of Antibodies used in the study

| Antibody | Vendor | Cat.No. | Clone | Dilution |
|---|---|---|---|---|
| R-PE anti-CD31 | Ancell | 180-050 | 158-2B3 | 1:100 (FC) |
| APC anti-CD31 | Biolegend | 303116 | WM-59 | 1:100 (FC) |
| PE anti-CD144(VE-CAD) | ThermoFisher | 12-1449- | 16B1 | 1:100 (FC) |
| | | 80 | | |
| PE anti-TRA-1-81 | ThermoFisher | 12-8883- | | 1:100 (FC) |
| | | 80 | | |
| PE anti-human SSEA-4 Antibody | Biologend | 330405 | MC- | 1:100 (FC) |
| | | | 813-70 | |
| Rabbit anti-alpha smooth muscle actin | Abcam | Ab5694 | | 1:200(IF) |
| Mouse anti-alpha smooth muscle | Sigma | A2547 | 1A4 | 1:200(IF) |
| actin | | | | |
| Mouse anti-VE-cadherin | Santa Cruz | Sc-9989 | F-8 | 1:200(IF) |
| Rabbit anti-human Von Willebrand | DAKO | A0082 | | 1:200(IF) |
| Factor | | | | |
| Mouse anti-human CD31 | Agilent | M082329- | JC70A | 1:50(IHC-P) |
| | | 2 | | 1:200(IF) |
| Rhodamine labeled Ulex Europaeus | Vector | RL-1062 | (Human | 1:100(IHC- |
| Agglutinin I (UEA I) | | | specific) | P) |
| | | | | 1:200(IF) |
| Mouse anti-human vimentin | Abcam | Ab8069 | V9 | 1:300 |
| | | | | (IHC-P) |
| | | | (human | |
| | | | specific) | |
| Rabbit anti-Oct4 | Stemgent | 09-0023 | | 1:300(IF) |
| Rabbit anti-Sox2 | Stemgent | 09-0024 | | 1:300(IF) |
| Rabbit anti-Nanog | Stemgent | 09-0020 | | 1:300(IF) |
| | | | | 1:200 (IF) |
| Mouse anti-human Factor VIII | Green Mountain | GMA- | | 1:200 |
| | Antibodies | 8006 | | (WB) |
| Donkey anti-mouse IgG, AlexaFluor | ThermoFisher | A-21202 | | 1:500 (IF, |
| 488 | | | | IHC-P) |
| Donkey anti-goat IgG, AlexaFluor | ThermoFisher | A-11058 | | 1500 (IF, |
| 594 | | | | IHC-P) |
| Donkey anti-rabbit IgG, AlexaFluor | ThermoFisher | A-21206 | | 1500 (IF, |
| 488 | | | | IHC-P) |
| Horse anti-mouse IgG | Vector | TI-2000 | | 1:400 (IF, |
| | | | | IHC-P) |
| Texas Red | | | | |
| Horse anti-rabbit IgG | Vector | DI-1094 | | 1:400 (IF, |
| | | | | IHC-P) |
| DyLight 594 | | | | |

Differentiation of h-iPSCs into h-iECs

Basal medium for differentiation of HA-iPSCs into HA-iECs was prepared by adding 1× GlutaMax supplement (ThermoFisher, Cat No. 35050061) and 60 μg/mL L-Ascorbic acid (Sigma, Cat No. A8960) into Advanced DMEM/F12 (ThermoFisher, Cat No. 12634010). Culture medium for HA-iECs was prepared by mixing EGM-2 with 1× GutaMax supplement and 10 μM SB431542 (Selleckchem, Cat No. S1067).

For the differentiation, HA-iPSCs were dissociated with ReLeSR reagent (STEMCELL, Cat No. 05872) and plated on Matrigel at a density of 40,000 cells/cm² in mTeSR1 medium with 10 μM Y27632. After 24 h of allowing the cells to plate, the medium was changed to S1 medium consisting of basal medium supplemented with 6 μM CHR99021 (Sigma, Cat No. SML1046). After 48 h of culture in S1 media changed daily, h-MPCs were dissociated into single cells and then transfected with modRNA(ETV2) by electroporation. For electroporation, 2 million cells were resuspended in 100 μL buffer mixed with 0.8 μg modETV2. Electroporated cells were then seeded on a100-mm Matrigel-coated dish in S2 medium consisting of basal medium supplemented with 50 ng/mL VEGF-A (Peprotech, Cat No. 100-20), 50 ng/mL FGF-2, 10 ng/mL EGF and 10 μM SB431542. [Ref.: our ETV2 paper]

Genotyping of F8 c.6429+1G>A Mutation in the iPSCs-Derived from Patient #1

Genomic DNA (gDNA) was isolated from HA-iPSCs derived from patient #1 and control iPSCs. The junction region of Exon22 and Intron22 was amplified by PCR using primers listed in Table 11 (Exon 22 FWD1 and Intron 22 REV). The purified PCR product was then sequenced by Sanger method using Exon 22 FWD1 primer. In the control iPSCs, the first base of Intron22 (F8 c.6429+1) is G. In HA-iPSCs derived from patient #1, a point G>A mutation should be detected at this position.

Genotyping of Type 1 Intron 22 Inversion Mutation in the iPSCs-Derived from Patients #5 and #6

mRNA was isolated and converted to cDNA from HA-iPSCs derived from patients #5,6 and control iPSCs. If the specimen carries Type 1 Intron 22 inversion mutation, the junction of Exon22 and Intron 22 will be amplified by PCR using primers—Exon 19 FWD1 and Intron 22 REV (PCR product size 378 bp; Table 12). The same primer set cannot amplify any fragment from cDNA of control iPSCs. On the other hand, primers —Exon 22 FWD1 and Exon 23 REV can amplify a 225-bp PCR product of Exon22-Exon23 junction from control iPSCs. However, the Exon22-Exon23 junction doesn't exist in iPSCs of Type 1 Intron 22 inversion mutation.

Modified mRNA Synthesis and Formulation

Chemically modified mRNA encoding ETV2 (modRNA(ETV2)) was generated by TriLink BioTechnologies, LLC. In brief, modRNA(ETV2) was synthesized in vitro by T7 RNA polymerase-mediated transcription from a linearized DNA template, which incorporates the 5′ and 3′ UTRs and a poly-A tail. Specifically, ETV2(NM_014209.3;

(SEQ ID NO: 1)
ORF:ATGGACCTGTGGAACTGGGATGAGGCATCCCCACAGGAAGTGCCTC
CAGGGAACAAGCTGGCAGGGCTTGAAGGAGCCAAATTAGGCTTCTGTTTC
CCTGATCTGGCACTCCAAGGGGACACGCCGACAGCGACAGCAGAGACATG
CTGGAAAGGTACAAGCTCATCCCTGGCAAGCTTCCCACAGCTGGACTGGG
GCTCCGCGTTACTGCACCCAGAAGTTCCATGGGGGGCGGAGCCCGACTCT
CAGGCTCTTCCGTGGTCCGGGGACTGGACAGACATGGCGTGCACAGCCTG
GGACTCTTGGAGCGGCGCCTCGCAGACCCTGGGCCCCGCCCCTCTCGGCC
CGGGCCCCATCCCCGCCGCCGGCTCCGAAGGCGCCGCGGGCCAGAACTGC
GTCCCCGTGGCGGGAGAGGCCACCTCGTGGTCGCGCGCCCAGGCCGCCGG
GAGCAACACCAGCTGGGACTGTTCTGTGGGGCCCGACGGCGATACCTACT
GGGGCAGTGGCCTGGGCGGGGAGCCGCGCACGGACTGTACCATTTCGTGG
GGCGGGCCCGCGGGCCCGGACTGTACCACCTCCTGGAACCCGGGGCTGCA
TGCGGGTGGCACCACCTCTTTGAAGCGGTACCAGAGCTCAGCTCTCACCG
TTTGCTCCGAACCGAGCCCGCAGTCGGACCGTGCCAGTTTGGCTCGATGC
CCCAAAACTAACCACCGAGGTCCCATTCAGCTGTGGCAGTTCCTCCTGGA
GCTGCTCCACGACGGGGCGCGTAGCAGCTGCATCCGTTGGACTGGCAACA
GCCGCGAGTTCCAGCTGTGCGACCCCAAAGAGGTGGCTCGGCTGTGGGGC
GAGCGCAAGAGAAAGCCGGGCATGAATTACGAGAAGCTGAGCCGGGGCCT
TCGCTACTACTATCGCCGCGACATCGTGCGCAAGAGCGGGGGGCGAAAGT
ACACGTACCGCTTCGGGGGCCGCGTGCCCAGCCTAGCCTATCCGGACTGT
GCGGGAGGCGGACGGGGAGCAGAGACACAATAA; 1029 bp)

was cloned into the mRNA expression vector pmRNA, which contains a T7 RNA polymerase promoter, an unstructured synthetic 5′ UTR, a multiple cloning site, and a 3′ UTR that was derived from the mouse α-globin 3′ gene. Co-transcriptional capping with CleanCap Cap1 AG trimer yields a naturally occurring Cap1 structure. 5′-triphosphate were removed to reduce innate immune response. Modified mRNA was dissolved in RNase-free sodium citrate buffer (1 mM, pH 6.4).

Purification and Expansion of h-iECs

At 48 hours after ETV2 electroporation, HA-iECs were dissociated with ReLeSR reagent (STEMCELL, Cat No. 05872) into single cells and sorted into CD31+ and CD31− cells using magnetic beads coated with anti-human CD31 antibodies (DynaBead, ThermoFisher, Cat No. 11155D). The purified CD31+ HA-iECs were then expanded in culture on 10-cm dishes coated with 1% gelatin and maintained in HA-iEC culture medium.

Electroporation

Electroporation was routinely used to introduce plasmids, modified mRNA, and proteins into the cells as described for each experiment. Electroporation was carried out with a Neon electroporation system (ThermoFisher). Unless specified otherwise, electroporation parameters were set as 1150 v for pulse voltage, 30 ms for pulse width, 2 for pulse number, 3 mL of electrolytic buffer, and 100 μL resuspension buffer R in 100 μL reaction tips (ThermoFisher, Cat No. MPK10096).

Construction of Full Length F8-Expressing PiggyBac Vector

A full-length factor 8 gene fragment was isolated from pCDNA4/Full length FVIII (Addgene, Plasmid #41036) through PCR with attB-F8 primers (Table 11) and subsequent gel isolation⁴. This fragment was inserted into a pDONR 221 vector (ThermoFisher, Cat. No. 12536017) through BP cloning using BP Clonase II enzyme mix (ThermoFisher, Cat. No. 11789020), then inserted into the pPB-PGK-destination vector (Addgene, Plasmid #60436) through LR cloning using LR Clonase enzyme mix (ThermoFisher, Cat. No. 11791019)⁵. The final construct PB-PGK-F8-Hyg contains a full-length F8 ORF driven by a CAG promoter and a hygromycin resistance gene driven by a PGK promoter, all flanked by 5′ and 3′ internal repeats (ITRs). A PiggyBac vector containing B domain-deleted FVIII (BDD-F8) was generated by the same method using pCDNA4/BDD-FVIII (Addgene, Plasmid #41035) as a PCR template⁴.

TABLE 11
Sequences of PCR primers

| Name/target | Sequence (5′→3′) | # |
|---|---|---|
| F8 attB Forward | GGGGACAAGTTTGTACAAAAAAGCA | 48 |
| | GGCTTAATGCAAATAGAGCTCTCCA | |
| | CCT | |
| F8 attB Reverse | GGGGACCACTTTGTACAAGAAAGCT | 49 |
| | GGGTTTCAGTAGAGGTCCTGTGCCT | |
| | CG | |
| BDD vs FL gel P1 | AGACTTTCGGAACAGAGGCA | 50 |
| BDD vs FL gel P2 | TTCTGTGTGCAAACCAAGGG | 51 |
| BDD vs FL gel P3 | GGCAAAGCAAGGTAGGACTG | 52 |
| BDD vs FL gel P4 | GAGCCCTGTTTCTTAGAACATG | 53 |
| Exon 22 FWD1 | GTGGATCTGTTGGCACCAATG | 54 |
| Exon 24 REV | CTCCCTTGGAGGTGAAGTCG | 55 |
| Exon 22 FWD2 | ACCAATGATTATTCACGGCATCAAGA | 56 |
| Exon 23 REV | TGCAAACGGATGTATCGAGCAATAA | 57 |
| Exon 19 FWD1 | TCCAAAGCTGGAATTTGGCG | 58 |
| Intron 22 REV | ACACAGTCCTGAATCACATA | 59 |

#, SEQ ID NO:

Establishment of HA-iPSC Line Expressing Full Length F8

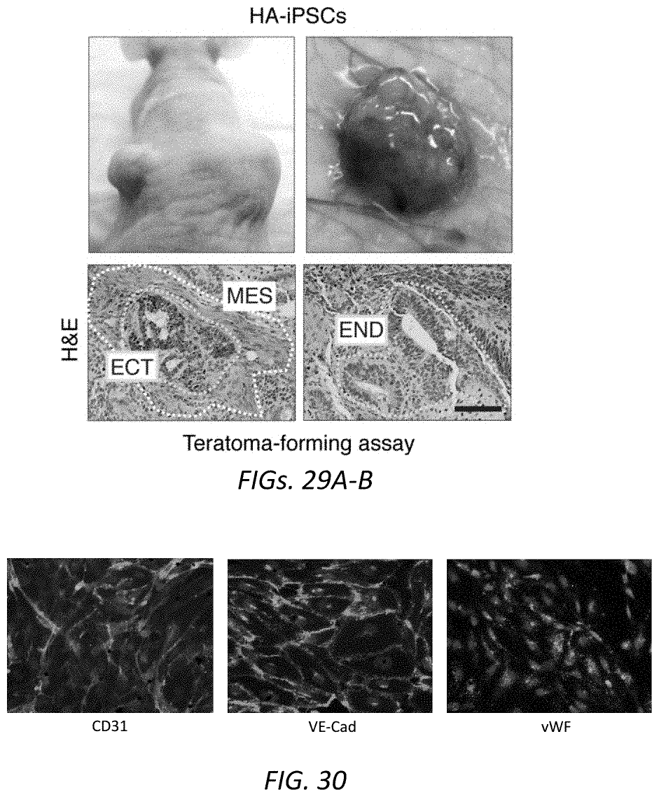

HA-iPSCs (clones from patient #1 with genotype F8 c.6429+1G>A) were dissociated and filtered through 40 m cell strainer to obtain a single cell suspension. For electroporation, 1 million HA-iPSCs were resuspended in 100 μL buffer mixed with 2.5 μg PB-PGK-F8-Hyg PiggyBac transposon vector and 0.5 μg super PiggyBac transposase expression vector (PB210PA-1, System Biosciences). The electroporated cells were then plated on a 35-mm Matrigel-coated dish in mTeSR1 medium with 10 μM Y27632. The expression of SPT will mobilize the transposon part of PB-PGK-F8-Hyg vector and insert them into TTAA sites on genome. After 24 hours, the culture medium was changed to mTeSR1 medium supplemented with 200 ug/mL Hygromycin B (ThermoFisher, Cat No. 10687010) and changed daily for 2 to 4 days until nontransfected cells were killed. Hygromycin selection was performed twice during expansion until all cells without PiggyBac integration were killed. Several F8 expressing HA-iPSC clones were isolated and cultured separately for further characterization. Upon clonal expansion, gene-edited HA-F8FL-iPSCs and HA-F8FL-iECs were characterized similarly to unedited HA-iPSCs and HA-iECs (FIG. 29-30). The expression of SPT at different time points after electroporation was measured by quantitative RT-PCR with specific primers (Table 11).

Verification of F8 Expression in HA-FLF8-iECs

To verify that the insertion of F8 in HA-FLF8-iECs corresponded to expression of a full length version of the gene, mRNA was isolated and converted to cDNA from HA-FLF8-iECs. Combinations of primers that recognize the transition between the A2 and B domains (P1-P2 primers; Table 11), between the B and A3 domains (P3-P4) of the F8 gene (FIG. 25B), and across the entire B domains (P1-P4) were used for PCR analysis. Complete F8 gene with a full B domain presence should reveal the presence of ˜245 bp DNA fragment for P1-P2 primers, ˜290 bp for P3-P4, and ˜3,083 bp for P1-P4. In contrast, control human ECs that were piggyBac transfected with a BDDF8 should have a ˜401 bp DNA fragment for P1-P4 and should lack fragments for P1-P2 and P3-P4 (FIG. 25B), as expected for a transgene lacking the B domain.

Measurement of PiggyBac Integration Copy Number

To test the number of PiggyBac insertions, HA-FLF8-iPSC clonal cell pellets from culture were first lysed. With the lysate, a quantitative PCR based system was used to measure the transposon copy number relative to a genomic counting primer set. We used reagents and primers provided by the PiggyBac qPCR Copy Number Kit according to the manufacturer's instructions (SBI, Cat No. PBC100A-1).

Generation of Immunodeficient Hemophilia a Mouse Model for Human Cell Engraftment

B6; 129S-F8tm1Kaz/J (FVIIIKO) mice were purchased from The Jackson Laboratory.

These mice are homozygous for the targeted, X chromosome-linked F8 mutant allele and they are viable and fertile. Homozygous females and carrier males have less than 1% of normal factor VIII activity and exhibit prolonged clotting times.

Immunodeficient hemophilic (FVIIIKO-SCID) mice were developed by crossing FVIIIKO female mice with NOD.SCID male mice (NOD.CB17-Prkdcscid/J). The F1 mice then crossed with each other to generate F2 progenies. Within F2, homozygous females (Prkdc^{scid/scid} F8^{-/-}) and carrier males (Prkdc^{scid/scid} F8^{-/Y}) were screened out by genotyping (performed by Transnetyx Inc) and crossed for 6 additional generations to obtain a stable line (FIGS. 29A-B). No bleeding difficulties are apparent during birth^{6,7}. These transgenic mice have less than 1% of normal factor VIII activity (as the FVIIIKO mice) and they are unable to mount a specific immune response to foreign antigens (as the SCID mice). Thus, they do not generate inhibitory antibodies against FVIII. These mice recapitulate key features of hemophilia A and are immunodeficient, and thus provide an excellent model for use in exploring our gene therapy strategy with FVIII-secreting human implants.

In Vivo Vasculogenic Assay

FVIIIKO-SCID mice (6 to 12 weeks) were housed in compliance with Boston Children's Hospital guidelines, and all animal-related protocols were approved by the Institutional Animal Care and Use Committee. Vasculogenesis was evaluated in vivo using our xenograft model as previously described [Ref R Z Lin, Methods 56 (2012) 440-451]. Briefly, h-iECs and MSCs (2M total; 2:3 ECFC/MSC ratio) were resuspended in 200 of collagen/fibrin/laminin-based solution (1.5 mg/mL of bovine collagen (Trevigen, Cat No. 3442-050-01), 2 mg/mL of laminin-1, 30 ug/mL of human fibronectin, 25 mM HEPES, 10% 10×DMEM, 10% FBS, 5 μg/mL of EPO (ProSpec, Cat No. CYT-201), 1 μg/mL of FGF2 (Peprotech, Cat No. 100-18B), and 3 mg/mL of fibrinogen, pH neutral). Before cell injection, 50 uL of 10 U/mL thrombin was subcutaneously injected.

Histology and Immunofluorescence Staining

Explanted grafts were fixed overnight in 10% buffered formalin, embedded in paraffin, and sectioned (7-μm-thick). Microvessel density was reported as the average number of erythrocyte-filled vessels (vessels/mm²) in H&E-stained sections from the middle of the implants as previously described⁹ (Ref. R Z Lin, Methods 56 (2012) 440-451). For immunostaining, sections were deparaffinized and antigen retrieval was carried out with citrate buffer (10 mM sodium citrate, 0.05% Tween 20, pH 6.0). Sections were then blocked for 30 min in 5% horse serum and incubated with primary antibodies overnight at 4° C. The sections were then incubated with fluorescently-conjugated secondary antibodies for 1 hour followed by DAPI counterstaining. Human-specific anti-CD31 antibody and UEA-1 lectin were used to stain human blood vessels. Perivascular cells were stained by anti-alpha smooth muscle actin antibody. Primary and secondary antibodies are detailed in Table 12.

Tail Clip Bleeding Assay and Blood Plasma Analysis

Mice were anesthetized with ketamine/xylazine at 100-120 mg/kg. When the animal was no longer moving involuntarily, it was weighed then placed on a paper towel in a prone position. A distal 10-mm segment of the tail was amputated with a scalpel. The tail was immediately immersed in a 50-mL Falcon tube containing isotonic saline pre-warmed in a water bath to 37° C. The position of the tail was vertical with the tip positioned about 2 cm below the body horizon. Each animal was monitored for 20 min even if bleeding ceased, in order to detect any re-bleeding. Bleeding time was determined using a stop clock. If bleeding on/off cycles occurred, the sum of bleeding times within the 20-min period was used. The assay terminated at the end of 20 min. Body weight, including the tail tip, was measured again, and the volume of blood loss during the experimental period was estimated from the reduction in body weight. At the end of experiment, animals were euthanized with CO2, and 0.5 mL of blood was collected from the heart to collect blood plasma supplemented with 10% sodium citrate to avoid clotting. This plasma was then analyzed for FVIII activity using the Chromogenix Coamatic Factor VIII assay (diapharma, Cat No. K822585) according to manufacturer's instructions with recombinant protein (Kogenate, Bayer) as a standard curve control.

Flow Cytometry

Cells were dissociated into single-cell suspensions using TrypLE and washed with PBS supplemented with 1% BSA and 0.2 mM EDTA. In indicated experiments, cells were stained with flow cytometry antibodies and analyzed using a Guava easyCyte 6HT/2L flow cytometer (Millipore Corporation, Billerica, Mass.) and FlowJo software (Tree Star Inc., Ashland, Oreg.). Antibody labeling was carried out for 10 min on ice followed by 3 washes with PBS buffer. Antibody information is detailed in Table 12.

Immunofluorescence Staining of Cells in Culture

Cells were seeded in LAB-TEK chamber slides. After confluency, cells were fixed in 4% paraformaldehyde (PFA), permeabilized with 0.1% Triton X-100 in PBS, and then blocked for 30 min in 5% horse serum (Vector, Cat No. S-2000). Subsequently, cells were incubated with primary antibodies for 1 hour at room temperature (RT). Cells were washed 3 times with PBS and then incubated with secondary antibodies for 1 hour at RT. Cells were washed 3 times with PBS and stained with 0.5 μg/mL DAPI for 10 min. Slides were mounted with DAKO fluorescence mounting medium (Agilent, Cat No. S302380-2). Antibody information is detailed in Table 12.

Microscopy

Images were taken using an Axio Observer Z1 inverted microscope (Carl Zeiss) and AxioVision Rel. 4.8 software. Fluorescent images were taken with an ApoTome 2. Optical sectioning system (Carl Zeiss) and 20×objective lens. Non-fluorescent images were taken with an AxioCam MRc5 camera using a 5× or 10× objective lens.

Quantitative RT-PCR

Quantitative RT-PCR (qRT-PCR) was carried out in RNA lysates prepared from cells in culture. Total RNA was isolated with a RNeasy kit (Qiagen, Cat No. 74106) and cDNA was prepared using reverse transcriptase III (ThermoFisher, Cat No. 4368814), according to the manufacturer's instructions. Quantitative PCR was performed using SRBR Green Master Mix (ThermoFisher, Cat No. A25776), and detection was achieved using the StepOnePlus Real-time PCR system thermocycler (Applied Biosystems). Expression of target genes was normalized to GAPDH. Real-time PCR primer sequences are listed in Table 10.

TABLE 10
Sequences of qPCR primers

| Gene | Forward (5′→3′) | # | Reverse (5′→3′) | # |
|---|---|---|---|---|
| F8-P1 | CCAGAATCAGCAAGGTGGAT | 60 | AGGTTTCTGCTGCTTGGAAA | 61 |
| F8-P2 | CACTCTTCGCATGGAGTTGA | 62 | AGTCCACTTGCAGCCACTCT | 63 |
| SPT | CAGAGAACCATCAGAGGCAAG | 64 | TCACCAGGATGCCGAAGAAG | 65 |
| GAPDH | CATGTTCGTCATGGGTGTGAA | 66 | ATGGCATGGACTGTGGTCAT | 67 |
| | CCA | | GAGT | |

#, SEQ ID NO:

Statistical Analyses

Unless otherwise stated, data were expressed as mean standard deviation of the mean (s.d.). For comparisons between two groups, means were compared using unpaired two-tailed Student's t-tests. Comparisons between multiple groups were performed by ANOVA followed by Bonferroni's post-test analysis. Samples size, including number of mice per group, was chosen to ensure adequate power and were based on historical data. No exclusion criteria were applied for all analyses. No specific methods of randomization were applied to group/animal allocation. Investigators were not blinded to group allocation. All statistical analyses were performed using GraphPad Prism v.5 software (GraphPad Software Inc.). P<0.05 was considered statistically significant.

### Example 8A. Generation of HA-iPSCs and HA-iECs from Hemophilia A Patients

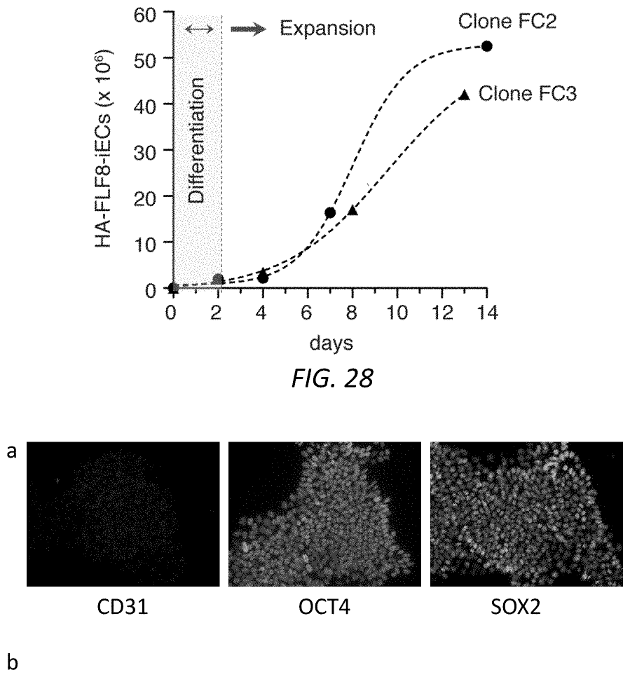

In order to bioengineer our FVIII-secreting implants, we developed a method to abundantly generate ECs from patients with hemophilia A. To this end, we followed an iPSC approach. In principle, human iPSCs could be generated from multiple donor cell types such as skin fibroblasts; however, acquiring cells from hemophilic patients is not trivial due to their bleeding disorder. Thus, to avoid invasive biopsies, we resorted to a protocol that uses exfoliated renal epithelial cells present in urine (cells referred to as HA-UECs)¹⁸. We isolated HA-UECs from urine collected from seven patients with severe hemophilia A (see FIG. 24A for a list of all patients' genotypes). HA-UECs were reprogrammed into HA-iPSCs via non-integrating episomal expression of selected reprogramming factors (FIG. 24B). Briefly, four plasmids encoding hOCT4, hSOX2, hKLF4, hL-MYC, hLIN-28 and EBNA-1 were introduced via electroporation into HA-UECs. Subsequently, HA-iPSC colonies spontaneously emerged between days 14-21 (FIG. 24C). Colonies were then transferred to Matrigel-coated, feeder-free culture plates for expansion. HA-iPSCs were highly pure (FIG. 24E) and their phenotypes were validated by expression of pluripotent transcription factors OCT4, NANOG, and SOX2 (FIG. 24D); lack of CD31 expression (FIG. 24D); and by the ability to form teratomas in mice (FIG. 24F). Moreover, patient specificity of the resulting HA-iPSCs was confirmed by analysis of HA-iPSC DNA at the specific mutation—FIG. 24G depicts two examples of HA-iPSC patient-specificity corresponding to 1) sequencing of a single G>A point mutation using amplified gDNA(FIG. 24G, left), and 2) an intron 22 inversion utilizing PCR of cDNA for presence of an intron confirming an inversion(FIG. 24G, right)¹⁹. Next, we differentiated patients' HA-iPSCs into HA-iECs. To this end, we used a two-dimensional, feeder-free, and chemically defined protocol recently developed by our group [Our ETV2 paper]. Briefly, HA-iPSCs were first converted into intermediate mesodermal progenitor cells via activation of Wnt signaling, a step that lasts 48 h. Thereafter, mesodermal progenitor cells were electroporated and exposed to chemically modified RNA (modRNA) encoding the transcription factor ETV2 [Our ETV2 paper](FIG. 24B). This two-step protocol rapidly and uniformly converted HA-iPSCs into HA-iECs (FIG. 24C). Indeed, 48 h after transfection with modRNA(ETV2), HA-iECs uniformly expressed endothelial markers VE-Cadherin, CD31, and vWF; and lacked expression of pluripotent markers OCT4, SSEA4, and Tra-1-81 (FIG. 24H-J). Moreover, this protocol enabled a high degree of reproducibility and independent clones generated from a single HA-iPSC line consistently yielded HA-iECs with purity between 75-97% (quantified by dual CD31 and VE-Cadherin expression), which was similar to the purity of differentiation in non-hemophilic human iPSC clones (FIG. 24H). Lastly, HA-iECs were easily expanded in culture for a period of 2 weeks, with an average expansion yield of >40-fold (FIG. 28), which was deemed more than sufficient to obtain the necessary cells for our grafts.

### Example 8B. Stable Expression of Full-Length FVIII in HA-iECs by piggyBac Vectors

In order to achieve stable expression of FVIII in HA-iECs, we used a non-viral piggyBac DNA transposon system. The strategy was first to transduce HA-iPSCs, and then select clones with high-level transgene expression of FLF8. The selected HA-FLF8-iPSC clones were subsequently differentiated into HA-FLF8-iECs using our modRNA (ETV2) method (FIG. 25A). Our piggyBac system was comprised of two separate vectors. First, we constructed a 14.4 kb transposon vector with expression of human FLF8 under a CAG promoter and a hygromycin resistance gene driven by PGK between to ITRs for genome insertion. This transposon vector was combined with a 7 kb plasmid encoding a super piggyBac transposase under a CMV promoter. The two vectors were combined at a 5:1 ratio (transposon:transposase), in line with previous reports²⁰, and electroporated into HA-iPSCs. After two rounds of selection, clonal HA-FLF8-iPSCs were analyzed and were shown to retain stem cell properties and maintain their ability to form teratomas and differentiate into HA-FLF8-iECs with uniform endothelial marker expression (FIG. 29a-c). Moreover, we demonstrated that transposase activity remained negligible after HA-FLF8-iPSC cloning and after differentiation into HA-FLF8-iECs. We also showed that F8 transgene copy number remained stable after multiple passages in culture.

We then verified that insertion of F8 in the resulting HA-FLF8-iECs corresponded to expression of a full-length version of the gene (FIG. 25B) using a combination of primers that recognize the transition between A2 and B (BDD vs FL gel P1-P2 primers; Table S2) and between B and A3 (P3-P4) domains of the F8 gene (FIG. 25B). Indeed, PCR analysis of cDNA from HA-FLF8-iECs revealed the presence of ˜245 bp DNA fragment for P1-P2 primers, ˜290 bp for P3-P4, and ˜3083 bp (P1-P4), which is consistent with a full B domain presence. In contrast, control human ECs that were piggyBac transduced with a BDD-F8 (i.e., lacked B domain) had a ˜401 bp DNA fragment for P1-P4 and lacked fragments for P1-P2 and P3-P4 (FIG. 25B). It is important to note that for these experiments, we used HA-iECs derived from a cross-reacting material-positive (CRM+) patient (genotype: F8 c.6429+1G>A). In principle, CRM+ patients could have positive intracellular mRNA expression for endogenous FVIII. However, we found that unedited iPSC-derived HA-iECs had a virtually undetectable expression of endogenous F8 expression compared to the genetically edited ones (FIG. 25B).

Next, we examined the level of F8 expression in HA-FLF8-iECs derived from 5 independent HA-FLF8-iPSC clones (FIG. 25C, 25D). All clones were derived from the same HA-FLF8-iPSC line (genotype: F8 c.6429+1G>A) displayed high-level transgene expression at the mRNA level, ranging from 20-370-fold increase compared to the unedited HA-iPSC and HA-iEC controls (both from the same parental HA-iPSC line) (FIG. 25C). Moreover, each of the five HA-iPSC clones contained multiple piggyBac insertions (ranging from 8-160; measured by qPCR), and there was a linear correlation (R²=0.79) between the number of insertions detected in a HA-FLF8-iPSC clone and the level of transgene expression in the corresponding HA-FLF8-iECs (FIG. 25D). The presence of multiple insertions is one of the advantages of the piggyBac system and enables higher levels of transgene expression.

Lastly, expression of FVIII was also corroborated in HA-FLF8-iECs at the protein level (FIG. 25E-25F). The level of FVIII expression in HA-FLF8-iECs was significantly upregulated compared to unedited HA-iECs. Of note, immunofluorescent analysis revealed that the pattern of FVIII expression was punctuated and partially co-localized with vWF (FIG. 25F), which is consistent with FVIII storage into Weibel-Palade bodies in ECs²¹.

### Example 8C. Bioengineering Hemophilia A Patient-Specific FVIII-Secreting Vascular Networks in Hemophilic Mice

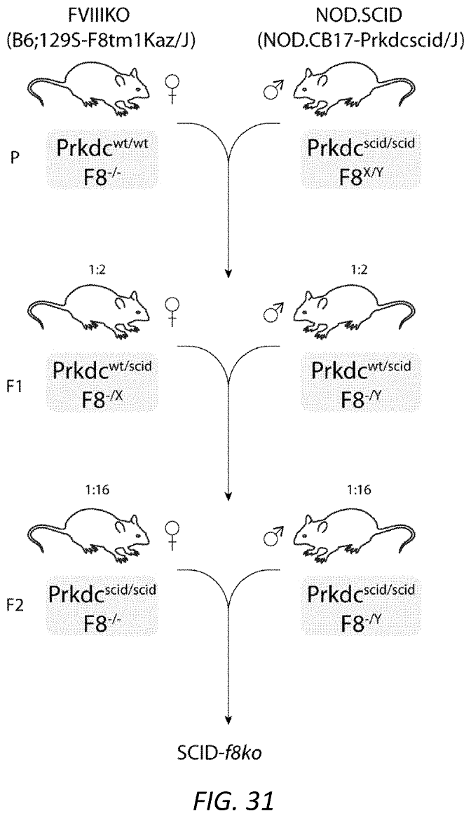

Next, we examined the capacity of HA-FLF8-iECs to engraft as functional blood vessels in vivo (FIG. 26a-f). To this end, we used a bioengineering approach that we previously developed to generate implants with human vascular networks in immunodeficient mice ²². Briefly, we prepared grafts by mixing a suspension of HA-FLF8-iECs (derived from clone F8-C4 in FIG. 25C-D; parental HA-iPSC line genotype: F8 c.6429+1G>A) and human mesenchymal stem cells (MSCs) (2×10⁶ cells; 1:1.5 ratio respectively) in a collagen/fibrin hydrogel that was previously shown to be compatible with vascular morphogenesis²³. Implants containing unedited patient-derived HA-iECs served as a control. The hydrogel-cell mixtures were then subcutaneously injected into hemophilic SCID-f8ko mice, effectively creating easily identifiable and accessible implants (FIG. 26A). We used SCID-f8ko mice generated in our laboratory by crossing f8ko mice (B6; 129S-F8tm1Kaz/J from The Jackson Laboratory, which contain a targeted mutation that disrupts exon 16 in the murine f8 gene) with NOD.SCID mice over several generations (FIG. 31). The resulting mice were genotyped, and their bleeding disorder was validated by the standard tail-tip bleeding assay.

We examined our implants after 7 days in vivo. Macroscopic observation of the explants suggested similarities in the degree of vascularization between implants containing HA-FLF8-iECs (n=10) or unedited HA-iECs (n=5) (FIG. 26A). Histological (H&E) analysis revealed that grafts from both groups had extensive networks of perfused microvessels (FIG. 26B), with similar microvessel densities (FIG. 26C). These microvessels were primarily lined by the HA-iECs, as confirmed by the expression of human-specific CD31 (FIG. 26D). Moreover, these human lumens contained mouse erythrocytes, indicating formation of functional anastomoses with the host circulatory system. We also examined the presence of mural cell investment around the newly-formed human vessels, a hallmark of proper vessel maturation and stabilization¹³. Immunohistological analyses revealed that the large majority (˜81-100%) of the human vessels in grafts from both groups had proper coverage by perivascular cells expressing α-smooth muscle actin (α-SMA) (FIG. 26D-E). This high percentage of perivascular coverage is to be expected by day 7 in this model¹⁴, and we have previously shown that vessels formed by control (non-hemophilic) iECs consistently displayed high degree of mural coverage (See Examples 1-7).

Importantly, HA-FLF8-iECs maintained expression of FVIII upon engraftment. Indeed, the expression of FVIII was significantly different between implants containing HA-FLF8-iECs or unedited HA-iECs. In grafts formed with HA-FLF8-iECs, human microvessels—identified by expression of h-CD31—displayed noticeable expression of FVIII at their lumens (FIG. 26F). In contrast, expression of FVIII in human microvessels lined by the unedited HA-iECs was virtually undetectable. Collectively, these results show that the genetically-engineered HA-FLF8-iECs were able to engraft in the form of functional, perfused vascular networks and that they retained their ability to over-express FVIII in vivo.

### Example 8D. Secretion of FL-FVIII into the Bloodstream and Correction of Coagulation Deficiency in Hemophilic Mice

We next sought to determine whether our subcutaneous microvascular grafts were able to effectively release functional full-length FVIII into the bloodstream of the implant-bearing mice, and whether the amount released was sufficient to correct their bleeding deficiency. To address these questions, we subjected each implant-bearing mouse to a standardized tail bleeding assay in which a distal 10-mm segment of the tail is amputated to assess bleeding and coagulation (FIG. 27A)²⁴. Animals with implants containing HA-FLF8-iECs or unedited HA-iECs were monitored for 20 min upon tail tip amputation, even if bleeding ceased, in order to detect any re-bleeding. If bleeding on/off cycles occurred, the sum of bleeding times within the 20-min period was used. The assay was terminated at the end of 20 min, and body weight (including the tail tip), was measured to determine the percent body weight change from blood loss. SCID and SCID-f8ko mice bearing no implants served as controls for normal and hemophilic bleeding, respectively. Using this approach, we demonstrated that implants containing HA-FLF8-iECs for 7 days were able to correct the clotting deficiency of the hemophilic animals and significantly decrease both percent body weight change and bleeding time compared to the unedited HA-iEC implant mice. Of note, the healthy SCID and SCID-f8ko with HA-FLF8-iEC implants had similarly low body weight loss and bleeding times during the bleeding assay (FIG. 27B-C). In order to further quantify the presence of non-mutant FVIII released by our implants, we also collected blood plasma from all implant-bearing mice at day 7. Blood plasma was then analyzed for FVIII activity using the Cromogenix Coamatic Factor VIII assay (diapharma, Cat No. K822585) with recombinant BBD-FVIII (Kogenate®; Bayer) as a standard control. In mice containing HA-FLF8-iEC implants, there was a significant increase in FVIII activity that was on average 6 times higher (˜6 IU/mL) than the healthy SCID controls (˜1 IU/mL) suggesting highly efficient release of protein from our implants (FIG. 27D).

Of note, the capacity to secrete functional FVIII by HA-FLF8-iECs was only observed in vivo. In contrast, in vitro, we did not find differences in FVIII secretion between the edited HA-FLF8-iECs and the unedited HAiECs (Supplemental FIG. 8), which suggested the importance of having HA-FLF8-iECs assembled in a proper blood vessel configuration.

Taken together these results show significant restoration of hemostasis and validate the proof-of-concept that our bioengineered microvessels can produce and secrete functional FVIII, restoring therapeutic levels of FVIII activity and treating hemophilia A.

### REFERENCES FOR EXAMPLE 8

1. Gitschier, J. et al. Detection and sequence of mutations in the factor VIII gene of haemophiliacs. Nature 315, 315427a0 (1985).
2. Soucie, J. et al. Mortality among males with hemophilia: relations with source of medical care. The Hemophilia Surveillance System Project Investigators. Blood 96, 437-42 (2000).
3. Gouw, S. C. et al. Factor VIII Products and Inhibitor Development in Severe Hemophilia A. The New England Journal of Medicine 368, 231-239 (2013).
4. Barr, R. D. et al. Health status and health-related quality of life associated with hemophilia. American Journal of Hematology 71, 152-160 (2002).
5. von Mackensen, S., Gringeri, A., Siboni, Mannucci, P. & (AICE), I. Health-related quality of life and psychological well-being in elderly patients with haemophilia.

Haemophilia 18, 345-352 (2012).
6. Lindvall, K. et al. Compliance with treatment and understanding of own disease in patients with severe and moderate haemophilia. Haemophilia 12, 47-51 (2006).
7. Walsh, C. & Valentino, L. Factor VIII prophylaxis for adult patients with severe haemophilia A: results of a US survey of attitudes and practices. Haemophilia 15, 1014-1021 (2009).
8. High, K. A. The gene therapy journey for hemophilia: are we there yet? Blood 120, 4482-4487(2012).
9. Mätrai, J., Chuah, M. K. & VandenDriessche, T. Preclinical and clinical progress in hemophilia gene therapy. Curr Opin Hematol 17, 387-392 (2010).
10. Hu, C., Cela, R. G., Suzuki, M., Lee, B. & Lipshutz, G. S. Neonatal helper-dependent adenoviral vector gene therapy mediates correction of hemophilia A and tolerance to human factor VIII. Proceedings of the National Academy of Sciences 108, 2082-2087 (2011).
11. Brown, B. D. et al. Helper-dependent adenoviral vectors mediate therapeutic factor VIII expression for several months with minimal accompanying toxicity in a canine model of severe hemophilia A. Blood 103, 804-810 (2004).
12. Sarkar, R. et al. Total correction of hemophilia A mice with canine FVIII using an AAV 8 serotype. Blood 103, 1253-1260 (2004).
13. Jiang, H. et al. Multiyear therapeutic benefit of AAV serotypes 2, 6, and 8 delivering factor VIII to hemophilia A mice and dogs. Blood 108, 107-115 (2006).
14. Lu, H. et al. Complete correction of hemophilia A with adeno-associated viral vectors containing a full-size expression cassette. Human gene therapy 19, 648-54 (2008).
15. PIPE, S. Functional roles of the factor VIII B domain. Haemophilia 15, 1187-1196 (2009).
16. Roth, D. et al. Nonviral Transfer of the Gene Encoding Coagulation Factor VIII in Patients with Severe Hemophilia A. The New England Journal of Medicine 344, 1735-1742 (2001).
17. Lin, Y. et al. Use of blood outgrowth endothelial cells for gene therapy for hemophilia A. Blood 99, 457-462 (2002).
18. Zhou, T. et al. Generation of human induced pluripotent stem cells from urine samples. Nat Protoc 7, 2080 (2012).
19. Dutta, D., Gunasekera, D., Ragni, M. V. & Pratt, K. P. Accurate, simple, and inexpensive assays to diagnose F8 gene inversion mutations in hemophilia A patients and carriers. Blood Advances 1, 231-239 (2016).
20. Matsui, H. et al. Delivery of Full-Length Factor VIII Using a piggyBac Transposon Vector to Correct a Mouse Model of Hemophilia A. Plos One 9, e104957 (2014).
21. Turner, N. A. & Moake, J. L. Factor VIII Is Synthesized in Human Endothelial Cells, Packaged in Weibel-Palade Bodies and Secreted Bound to ULVWF Strings. Plos One 10, e0140740 (2015).
22. Melero-Martin, J. M. et al. Engineering Robust and Functional Vascular Networks In Vivo With Human Adult and Cord Blood-Derived Progenitor Cells. Circ Res 103, 194-202(2008).
23. Lin, R.-Z. et al. Human endothelial colony-forming cells serve as trophic mediators for mesenchymal stem cell engraftment via paracrine signaling. Proc National Acad Sci 111, 10137-10142 (2014).
24. Liu, Y., Jennings, N. L., rt, A. & Du, X.-J. Standardizing a simpler, more sensitive and accurate tail bleeding assay in mice. World J Exp Medicine 2, 30 (2012).
25. Rangarajan, S. et al. AAV5-Factor VIII Gene Transfer in Severe Hemophilia A. New Engl J Medicine 377, 2519-2530 (2017).
26. Perrin, G. Q., Herzog, R. W. & Markusic, D. M. Update on clinical gene therapy for hemophilia. Blood 133, 407-414 (2018).
27. Thomas, C. E., Birkett, D., Anozie, I., Castro, M. G. & Lowenstein, P. R. Acute Direct Adenoviral Vector Cytotoxicity and Chronic, but Not Acute, Inflammatory Responses Correlate with Decreased Vector-Mediated Transgene Expression in the Brain. Mol Ther 3, 36-46 (2001).
28. LIRAS, A. & OLLLAS, S. Gene therapy for haemophilia . . . yes, but . . . with non-viral vectors? Haemophilia 15, 811-816 (2009).
29. Wilson, M. H., Coates, C. J. & George, A. L. PiggyBac Transposon-mediated Gene Transfer in Human Cells. Molecular Therapy 15, 139-145 (2007).
30. Chen, Y. et al. Functional Human Vascular Network Generated in Photocrosslinkable Gelatin Methacrylate Hydrogels. Adv Funct Mater 22, 2027-2039 (2012).
31. Lin, R.-Z., Chen, Y.-C., Moreno-Luna, R., Khademhosseini, A. & Melero-Martin, J. M. Transdermal regulation of vascular network bioengineering using a photopolymerizable methacrylated gelatin hydrogel. Biomaterials 34, 6785-6796 (2013).
32. Lin, R.-Z. et al. Host non-inflammatory neutrophils mediate the engraftment of bioengineered vascular networks. Nat Biomed Eng 1, s41551-017-0081 (2017).
33. Lin, R.-Z. et al. Induction of erythropoiesis using human vascular networks genetically engineered for controlled erythropoietin release. Blood 118, 5420-5428 (2011).
34. Xu, D. et al. Phenotypic correction of murine hemophilia A using an iPS cell-based therapy. Proc National Acad Sci 106, 808-813 (2009).
35. Olgasi, C. et al. Patient-Specific iPSC-Derived Endothelial Cells Provide Long-Term Phenotypic Correction of Hemophilia A. Stem Cell Rep 11, 1391-1406 (2018).
36. Ozelo, M. C. et al. Omental implantation of BOECs in hemophilia dogs results in circulating FVIII antigen and a complex immune response. Blood 123, 4045-4053 (2014).
37. Park, C.-Y. et al. Functional Correction of Large Factor VIII Gene Chromosomal Inversions in Hemophilia A Patient-Derived iPSCs Using CRISPR-Cas9. Cell Stem Cell 17, 213-220 (2015).
38. SHI, Q. et al. Lentivirus-mediated platelet-derived factor VIII gene therapy in murine haemophilia A. Journal of Thrombosis and Haemostasis 5, 352-361 (2007).
39. Shi, Q. et al. Syngeneic transplantation of hematopoietic stem cells that are genetically modified to express factor VIII in platelets restores hemostasis to hemophilia A mice with preexisting FVIII immunity. Blood 112, 2713-2721 (2008).
40. Shi, Q. et al. Platelet gene therapy corrects the hemophilic phenotype in immunocompromised hemophilia A mice transplanted with genetically manipulated human cord blood stem cells. Blood 123, 395-403 (2014).

### GENERAL REFERENCES

1. Aird, W. C. Endothelium in health and disease. Pharmacol Rep 60, 139-143 (2008).
2. Ding, B.-S. et al. Endothelial-derived angiocrine signals induce and sustain regenerative lung alveolarization. Cell 147, 539-553 (2011).
3. Paik, D. T. et al. Large-Scale Single-Cell RNA-Seq Reveals Molecular Signatures of Heterogeneous Populations of Human Induced Pluripotent Stem Cell-Derived Endothelial Cells. Circ Res 123, 443-450 (2018).
4. Patsch, C. et al. Generation of vascular endothelial and smooth muscle cells from human pluripotent stem cells. Nat Cell Biol 17, 994-1003 (2015).
5. Hu, S. et al. Effects of cellular origin on differentiation of human induced pluripotent stem cell-derived endothelial cells. JCI Insight 1, 915 (2016).
6. Chien, K. R., Zangi, L. & Lui, K. O. Synthetic chemically modified mRNA (modRNA): toward a new technology platform for cardiovascular biology and medicine. Cold Spring Harbor Perspectives in Medicine 5, a01403 5-a01403 5 (2014).
7. Harding, A. et al. Highly Efficient Differentiation of Endothelial Cells from Pluripotent Stem Cells Requires the MAPK and the PI3K Pathways. Stem Cells 35, 909-919(2017).
8. Sahara, M. et al. Manipulation of a VEGF-Notch signaling circuit drives formation of functional vascular endothelial progenitors from human pluripotent stem cells. Cell Res 24, 820-841 (2014).
9. Rasmussen, T. L. et al. ER71 directs mesodermal fate decisions during embryogenesis. Development 138, 4801-4812 (2011).
10. Kataoka, H. et al. Etv2/ER71 induces vascular mesoderm from Flk1+PDGFRα+ primitive mesoderm. Blood 118, 6975-6986 (2011).
11. Rasmussen, T. L. et al. VEGF/Flk1 signaling cascade transactivates Etv2 gene expression. PLoS ONE 7, e50103 (2012).
12. Elcheva, I. et al. Direct induction of haematoendothelial programs in human pluripotent stem cells by transcriptional regulators. Nat Commun 5, 4372 (2014).
13. Suknuntha, K. et al. Optimization of Synthetic mRNA for Highly Efficient Translation and its Application in the Generation of Endothelial and Hematopoietic Cells from Human and Primate Pluripotent Stem Cells. Stem Cell Rev 14, 525-534 (2018).
14. Lin, Y., Gil, C.-H. & Yoder, M. Differentiation, Evaluation, and Application of Human Induced Pluripotent Stem Cell-Derived Endothelial Cells. Arterioscler Thromb Vasc Biol 37, 2014-2025 (2017).
15. Melero-Martin, J. M. et al. Engineering robust and functional vascular networks in vivo with human adult and cord blood-derived progenitor cells. Circ Res 103, 194-202 (2008).
16. Stratman, A. N. et al. Interactions between mural cells and endothelial cells stabilize the developing zebrafish dorsal aorta. Development 144, 115-127 (2017).
17. Lin, R.-Z. et al. Host non-inflammatory neutrophils mediate the engraftment of bioengineered vascular networks. Nat. biomed. eng. 1, 0081 (2017).
18. Yamamizu, K., Kawasaki, K., Katayama, S., Watabe, T. & Yamashita, J. K.

Enhancement of vascular progenitor potential by protein kinase A through dual induction of Flk-1 and Neuropilin-1. Blood 114, 3707-3716 (2009).
19. Klein, D. iPSCs-based generation of vascular cells: reprogramming approaches and applications. Cell Mol Life Sci 75, 1411-1433 (2018).
20. Yang, L. et al. Human cardiovascular progenitor cells develop from a KDR+ embryonic-stem-cell-derived population. Nature 453, 524-528 (2008).
21. Vodyanik, M. A. et al. A mesoderm-derived precursor for mesenchymal stem and endothelial cells. Cell Stem Cell 7, 718-729 (2010).
22. Casie Chetty, S. et al. Vegf signaling promotes vascular endothelial differentiation by modulating etv2 expression. Dev Biol 424, 147-161 (2017).
23. Chen, Y. et al. Vascular endothelial growth factor promotes cardiomyocyte differentiation of embryonic stem cells. Am J Physiol Heart Circ Physiol 291, H1653-8 (2006).
24. Lee, J. H., Protze, S. I., Laksman, Z., Backx, P. H. & Keller, G. M. Human Pluripotent Stem Cell-Derived Atrial and Ventricular Cardiomyocytes Develop from Distinct Mesoderm Populations. Cell Stem Cell 21, 179-194.e4 (2017).
25. Han, S. et al. Generation of functional hepatic cells from pluripotent stem cells. J Stem Cell Res Ther Suppl 10, 1-7 (2012).
26. Oh, S.-Y., Kim, J. Y. & Park, C. The ETS Factor, ETV2: a Master Regulator for Vascular Endothelial Cell Development. Mol. Cells 38, 1029-1036 (2015).
27. Ferdous, A. et al. Nkx2-5 transactivates the Ets-related protein 71 gene and specifies an endothelial/endocardial fate in the developing embryo. Proc Natl Acad Sci USA 106, 814-819 (2009).
28. Lee, D. et al. ER71 acts downstream of BMP, Notch, and Wnt signaling in blood and vessel progenitor specification. Cell Stem Cell 2, 497-507 (2008).
29. Morita, R. et al. ETS transcription factor ETV2 directly converts human fibroblasts into functional endothelial cells. Proc Natl Acad Sci USA 112, 160-165 (2015).
30. Lee, S. et al. Direct Reprogramming of Human Dermal Fibroblasts Into Endothelial Cells Using ER71/ETV2. Circ Res 120, 848-861 (2017).
31. Yan, G. et al. Engineering vascularized skeletal muscle tissue with transcriptional factor ETV2-induced autologous endothelial cells. Protein Cell 122, 3982-222 (2018).
32. Le Bras, A. et al. Adventitial Sca1+ Cells Transduced With ETV2 Are Committed to the Endothelial Fate and Improve Vascular Remodeling After Injury. Arterioscler Thromb Vasc Biol 38, 232-244 (2018).
33. Lindgren, A. G., Veldman, M. B. & Lin, S. ETV2 expression increases the efficiency of primitive endothelial cell derivation from human embryonic stem cells. Cell Regen (Lond) 4, 1 (2015).
34. Lin, R.-Z. et al. Human endothelial colony-forming cells serve as trophic mediators for mesenchymal stem cell engraftment via paracrine signaling. Proc Natl Acad Sci USA 111, 10137-10142 (2014).
35. Melero-Martin, J. M. et al. In vivo vasculogenic potential of human blood-derived endothelial progenitor cells. Blood 109, 4761-4768 (2007).
36. Harding, A. et al. Highly Efficient Differentiation of Endothelial Cells from Pluripotent Stem Cells Requires the MAPK and the PI3K Pathways. Stem Cells 35, 909-919(2017).
37. Sahara, M. et al. Manipulation of a VEGF-Notch signaling circuit drives formation of functional vascular endothelial progenitors from human pluripotent stem cells. Cell Res 24, 820-841 (2014).
38. Patsch, C. et al. Generation of vascular endothelial and smooth muscle cells from human pluripotent stem cells. Nat Cell Biol 17, 994-1003 (2015).
39. Dobin, A. et al. STAR: ultrafast universal RNA-seq aligner. Bioinformatics 29, 15-21 (2013).
40. Garcia-Alcalde, F. et al. Qualimap: evaluating next-generation sequencing alignment data. Bioinformatics 28, 2678-2679 (2012).
41. Ewels, P., Magnusson, M., Lundin, S. & Kller, M. MultiQC: summarize analysis results for multiple tools and samples in a single report. Bioinformatics 32, 3047-3048 (2016).
42. Love, M. I., Huber, W. & Anders, S. Moderated estimation of fold change and dispersion for RNA-seq data with DESeq2. Genome Biol. 15, 550 (2014).

### Other Embodiments

It is to be understood that while the invention has been described in conjunction with the detailed description thereof, the foregoing description is intended to illustrate and not limit the scope of the invention, which is defined by the scope of the appended claims.

Other aspects, advantages, and modifications are within the scope of the following claims.

What is claimed is:

1. A method of generating induced endothelial cells, the method comprising:
providing a population of induced pluripotent stem cells (iPSCs) or human embryonic stem cells (h-ES cells);
incubating the iPSCs in media in the presence of a GSK3 inhibitor, under conditions sufficient for the iPSC to differentiate into intermediate mesodermal progenitor cells (MPCs);
optionally dissociating the MPCs into single cells;
introducing an exogenous nucleic acid encoding ETS translocation variant 2 (ETV2) to the MPCs to induce transient expression of exogenous ETV2; and
maintaining the MPCs under conditions sufficient for the MPCs to differentiate into iPSCs.

2. The method of claim 1, wherein the GSK3 inhibitor is CHIR99021, BIO, NP031112, IM-12; a pyrazolopyrimidine derivative, an analog of 7-hydroxy-1H-benzimidazole, a pyridinone, a pyrimidine, an indolylmaleimide analog, an imidazopyridine, an oxadiazole, a pyrazine, a thiadiazolidinone, amodin or 4-aminoethylamino emodin, or a 5-Imino-1,2,4-Thiadiazole (ITDZ).

3. The method of claim 1, wherein the iPSCs are incubated in in the presence of the GSK3 inhibitor for about 48 hours.

4. The method of claim 1, wherein the MPCs are incubated in media comprising (i) one or more growth factors, preferably selected from the group consisting of VEGF-A, FGF-2, and EGF, and (i) a TGFbeta receptor antagonist.

5. The method of claim 4, wherein the TGFbeta receptor antagonist is selected from the goup consisting of galunisertib (LY2157299 Monohydrate); A 83-01; RepSox; SD 208; SB 505124; LY 364947; D 4476; SB 525334; GW 788388; R 268712; IN 1130; SM 16; A 77-01; and SB431542.

6. The method of claim 1, wherein the MPCs are incubated in the media for about 48 hours after introduction of the ETV2 nucleic acid.

7. The method of claim 1, wherein the ETV2 nucleic acid comprises or encodes a sequence that is at least 95% identical to SEQ ID NO:1.

8. The method of claim 7, wherein the ETV2 nucleic acid is a synthetic, chemically modified mRNA, wherein at least one pseudouridine is substituted for uridine and/or at least one 5-methyl-cytosine is substituted for cytosine.

9. The method of claim 1, wherein the iPSCs are derived from a human primary cell.

10. The method of claim 1, further comprising maintaining the iECs in culture under conditions to allow for cell proliferation.

11. A population of iECs made by the method of claim 1.

12. A method of treating a subject in need of vascular cell therapy, comprising administering to the subject a therapeutically effective amount of the population of iECs of claim 11.

13. The method of claim 12, wherein the subject is in need of vascular cell therapy to treat ischemic or vascular injury and/or endothelial denudation, optionally in limbs, retina or myocardium; or for revascularization/neovascularization.

14. The method of claim 13, wherein the revascularization/neovascularization is to treat diabetes or promote success after organ transplantation.

15. A method of treating a subject who has hemophilia A or hemophilia B, the method comprising administering to the subject a therapeutically effective amount of the population of iECs of claim 11, wherein the iECs have been engineered to express Factor VIII or Factor IX.

16. The method of claim 15, wherein the cells are administered to the subject in a hydrogel.

17. The method of claim 16, wherein the hydrogel is administered by subcutaneous implantation.

18. A composition comprising a hydrogel and the population of iECs of claim 11.

19. The composition of claim 18, wherein the iECs have been engineered to express an exogenous protein.

20. The composition of claim 19, wherein the exogenous protein is Factor VIII or Factor IX.

21. The composition of claim 17, wherein the hydrogel comprises collagen and/or fibrin.

22. The composition of claim 21, wherein the hydrogel is a collagen/fibrin hydrogel or a crosslinked collagen hydrogel.

23. The method of claim 15, wherein engineering the cells to express a protein comprises introducing into the iECs a vector, preferably a transposon vector, for expression of the exogenous protein.
